(19) United States
(12) Patent Application Publication
(10) Pub. No.: US 2016/0200734 A1
(43) Pub. Date: Jul. 14, 2016

(54) CARBOXYLIC ACID COMPOUNDS IN TREATMENT OF DIABETES MELLITUS

(71) Applicants: Xinshan KANG, Beijing (CN); FUJIAN HAIXI PHARMACEUTICALS CO., LTD., Fuzhou, Fujian (CN)

(72) Inventors: Xinshan KANG, BEIJING (CN); Zhonghui CHEN, FUJIAN (CN)

(21) Appl. No.: 14/913,689

(22) PCT Filed: Aug. 22, 2014
(86) PCT No.: PCT/CN2014/084979

§ 371 Date: Feb. 23, 2016

(30) Foreign Application Priority Data
Aug. 23, 2013 (CN) 201310372674.X

(51) Int. Cl. C07D 495/04; C07D 491/048; C07D 409/12; C07D 333/60

(52) U.S. Cl. CPC C07D 495/04; C07D 333/60; C07D 491/048; C07D 409/12

(57) ABSTRACT

The invention relates to compounds for increasing QPR40 receptor activity and application thereof. These compounds include of a compound of Formula (I), pharmaceutically acceptable salts thereof, solvates thereof, isomers thereof, or produgs of the compounds mentioned above, or the mixture of any form above mentioned. The invention also relates to the use of the compounds in manufacturing a medicament for the treatment and/or prevention of diabetes, obesity or for Insulin secretagogue.

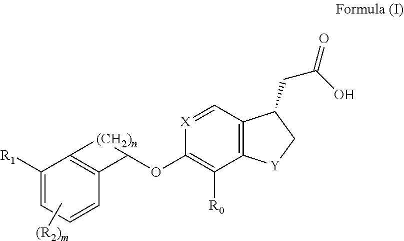

### BACKGROUND OF THE INVENTION

The present invention relates to carboxylic acid compounds for modulating GPR40 receptor function and their use in manufacturing a medicament.

Diabetes has become the third largest chronic noncommunicable diseases that serious threat to human health after tumor, cardiovascular disease, is a growing public health problem. The authority data released by the World Health Organization (WHO) show that the incidence of diabetes is growing rapidly worldwide in recent years. The number of diabetes patients has more than 177 million and is expected to reach 370 million by 2025. In view of the current serious situation, the development of new drugs to treat diabetes is very necessary.

GTP-binding protein coupling receptor 40 (GPR40) is a beta-cell 7 transmembrane receptor. The existing research results show that this new transmembrane receptors may be associated with certain types of cancer and neurological disease, especially diabetes. Analyses for the distribution of GPR40 in rat tissue demonstrated its highest expression level in the pancreas. Its expression was comparable to that of type A cholecystokinin receptor (CCKAR) and is one of the highly expressed receptors in pancreatic β cells. This suggesting that the GPR40 is a very important receptor in pancreatic β cells. When in the presence of high concentrations of glucose, FFAs amplify glucose-stimulated insulin secretion from Pancreatic β cells by activating GPR40. (Itoh Y, Kawamata Y, Harada M, et al. Free fatty acids regulate insulin secretion from pancreatic b cells through GPR40. Nature, 2003, 422 (6928): 173˜176)—Binding of FFA to GPR40 leads to IP₃(IP₃=InsP₃) production, activation of intracellular IP₃ receptors (IP₃R), and mobilization of intracellular Ca²⁺ from the endoplasmic reticulum (ER). GPR40 activation also stimulates Ca²⁺ influx through VDCC (voltage-dependent Ca²⁺-channels). The resulting increase in [Ca²⁺]ᵢ stimulates insulin secretion. Binding of FFA to GPR40 also produces an increase in intracellular cAMP levels, which antagonizes the activity of Kv channels further enhancing Ca²⁺ influx. (Jeper Gromada, The Free Fatty Acid Receptor GPR40 Generates Excitement in Pancreatic β-Cells. Endocrinology, February 2006, 147 (2):672-673)

Therefore, for those patients with insufficient insulin secretion, high efficient GPR40 agonists can be developed to stimulate insulin secretion from pancreatic β cells and reduce various diseases caused by insufficient insulin secretion. So far, few reports can be found on small molecule antagonist or agonist based on GPR40. To date, there is no medicine with GPR40 as targets in the market. The present invention provide a type of effective new compounds GPR40 agonists and therefore a medicine and method that safely and effectively increase insulin level.

### DETAILED DESCRIPTION OF THE INVENTION

The present invention provides carboxylic acid compounds for modulating GPR40 receptor function and their use in manufacturing a medicament for the treatment and/or prevention of diabetes, obesity, related disorders or for Insulin secretagogue.

These compounds include of a compound of Formula (I), pharmaceutically acceptable salts thereof, solvates thereof, isomers thereof, or produgs of the compounds mentioned above, or the mixture of any form above mentioned.

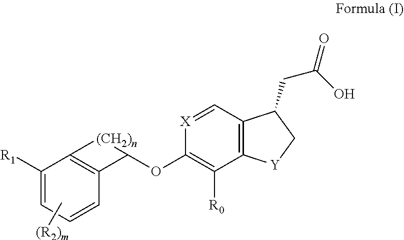

wherein

R₁ is selected from the group consisting of an aryl containing k substituents A₁, or a heteroaryl containing k substituents A₁;

Each A₁ is independently selected from —H, halogen, cyano, nitro, hydroxyl, lower alkyl, substituted lower alkyl, lower alkoxy, substituted lower alkoxy, cycloalkyl, substituted cycloalkyl, alkylsulfonylalkoxy, alkylthioalkoxy, alkoxyalkoxy or 1,1-dioxidotetrahydrothiopyranyloxy;

Each R₀ is selected from —H, halogen, lower alkyl, lower haloalkyl, substituted lower alkyl, lower alkoxy, substituted lower alkoxy, cycloalkyl, or substituted cycloalkyl;

R₂ is selected from —H, halogen, cyano, nitro, azyl, lower alkyl, lower haloalkyl, lower alkoxy, lower haloalkoxy, C₃-C₂₀ cycloalkyl, C₃-C₂₀ halocycloalkyl, C₆-C₁₄ aryl, C₇-C₁₆ aralkyl, C₆-C₁₄ aryloxy or C₇-C₁₆ aralkyloxy;

X is selected from C or N,

Y is selected from O or S,

m is an integer selected from 0, 1, 2 or 3;

n is an integer selected from 0, 2, 3, 4 or 5, and n is not 0 when both X is C and Y is S;

k is an integer selected from 1, 2, 3, 4 or 5.

In some embodiments, R₁ is selected from a C₆-C₂₀ aryl containing k substituents A₁, or a C₃-C₂₀ heteroaryl containing k substituents A₁. In some further embodiments, R₁ is selected from a C₆-C₁₆ aryl containing k substituents A₁, or a C₃-C₁₆ heteroaryl containing k substituents A₁. In yet embodiments, R₁ is selected from a C₆-C₁₂ aryl containing k substituents A₁, or a C₃-C₁₂ heteroaryl containing k substituents A₁ and any integer heteroatoms from 1 to 4. In yet further embodiments, R₁ is selected from a phenyl, naphthyl, biphenyl, anthryl, fluorenyl, pyrrolyl, furyl, thienyl, pyridyl, pyranyl, pyrazolyl, pyrimidyl, imidazolyl, thiazolyl, Oxazolyl, indolyl, carbazolyl, quinolyl, isoquinolyl, guanine, morpholinyl, piperazinyl, piperidyl, or pyrazinyl containing k substituents A₁. In a preferred embodiment of the present invention, R₁ is selected from a phenyl containing k substituents A₁.

In some embodiments, the compound and pharmaceutically acceptable salts or prodrugs thereof, wherein the compound is a compound of Formula (Ia).

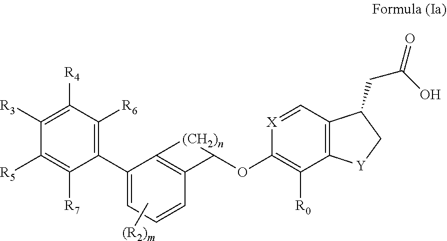

wherein,

R₂ is selected from —H, halogen, cyano, nitro, azyl, lower alkyl, lower haloalkyl, lower alkoxy, lower haloalkoxy, C₃-C₂₀ cycloalkyl, C₃-C₂₀ halocycloalkyl, C₆-C₁₄ aryl, C₇-C₁₆ aralkyl, C₆-C₁₄ aryloxy or C₇-C₁₆ aralkyloxy;

m is an integer selected from 0, 1, 2 or 3;

n is selected from any integer from 0, 2, 3, 4 or 5, and n is not 0 when both X is C and Y is S;

R₃ is selected from —H, halogen, cyano, nitro, lower alkyl, substituted lower alkyl, lower alkoxy, substituted lower alkoxy, cycloalkyl, substituted cycloalkyl, alkylsulfonylalkoxy, alkylthioalkoxy, alkoxyalkoxy or 1,1-dioxidotetrahydrothiopyranyloxy;

R₄, R₅, R₆ and R₇ are each independently selected from —H, halogen, cyano, nitro; or hydroxyl, hydroxymethyl, hydroxyethyl, 1-hydroxyethyl, hydroxypropyl, 1-hydroxypropyl, 2-hydroxypropyl, Hydroxyisopropyl, methyl, ethyl, propyl, isopropyl, n-butyl, isobutyl, tertbutyl, methoxyl, oxethyl, isopropoxy, butoxy, isobutoxy, tert-butoxy, sec-butoxy, azyl, dimethylamino, methylthio, sulfuryl, acetyl, cyclopropyl, cyclobutyl, cyclopentyl or cyclohexyl, optionally substituted by —F, —Cl or —Br;

R₀ is selected from —H, halogen, C₁-C₄ alkyl, C₁-C₄ haloalkyl, C₁-C₄ alkoxy, C₁-C₄ haloalkoxy, C₃-C₆ cycloalkyl or C₃-C₆ halocycloalkyl.

In some embodiments, R₃ is selected from —H, halogen, cyano, nitro, C₁₋₆ alkyl, C₁-C₆ haloalkyl, C₁-C₆ alkoxy, C₁-C₆ haloalkoxy, C₃-C₁₂ cycloalkyl, C₃-C₁₂ halocycloalkyl, C₁₋₆-alkylsulfonyl-C₁₋₆ alkoxy, C₁₋₆-alkylthio-C₁₋₆ alkoxy, C₁₋₆-alkoxy-C₁-C₆ alkoxy or 1,1-dioxidotetrahydrothiopyranyloxy. In some further embodiments. R₃ is selected from H, halogen, cyano, nitro, C₁-C₄ alkyl, C₁-C₄ haloalkyl, C₁-C₄ alkoxy, C₁-C₄ haloalkoxy, C₃-C₁₀ cycloalkyl, C₃-C₁₀halocycloalkyl, C₁₋₄ -alkylsulfonyl-C₁₋₄ alkoxy, C₁₋₄ -alkylthio-C₁₋₄ alkoxy, C₁₋₄ -alkoxy-C₁₋₄ alkoxy or 1,1-dioxidotetrahydrothiopyranyloxy. In yet further embodiments, R₃ is selected from —H, halogen, cyano, nitro; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl, cyclohexyl, methylsulfonylmethoxyl, methylsulfonyloxethyl, methylsulfonylpropoxy, methylsulfonylbutoxy, ethylsulfonylmethoxyl, ethylsulfonyloxethyl, ethylsulfonylpropoxy, ethylsulfonylbutoxy, methylthiomethoxyl, methylthiooxethyl, methylthiopropoxy, methylthiobutoxy, ethylthiomethoxyl, ethylthiooxethyl, ethylthiopropoxy, ethylthiobutoxy, methoxylmethoxyl, methoxyloxethyl, methoxylpropoxy, methoxyl butoxy, oxethylmethoxyl, oxethyloxethyl, oxethylpropoxy, oxethylbutoxy, or 1,1-dioxidotetrahydrothiopyranyloxy, optionally substituted by —F, —Cl or —Br. In a preferred embodiment of the present invention, R₃ is selected from —H, —F, —Cl, —Br, cyano, nitro, methyl, trifluoromethyl, methoxyl, trifluoromethoxy, cyclohexyl, methylsulfonylpropoxy, ethylsulfonylpropoxy, methylthiopropoxy, methoxylpropoxy or 1,1-dioxidotetrahydrothiopyranyloxy.

In some embodiments. R₃ is selected from —H, halogen, cyano, nitro, azyl, lower alkyl, lower haloalkyl, lower alkoxy, lower haloalkoxy, C₃-C₁₆ cycloalkyl, C₃-C₁₆ halocycloalkyl, C₆-C₁₂ aryl, C₇-C₁₂ aralkyl, C₆-C₁₂ aryloxy or C₇-C₁₂ aralkyloxy. In some further embodiments, R₂ is selected from —H, halogen, cyano, nitro, azyl, lower alkyl, lower haloalkyl, lower alkoxy, lower haloalkoxy, C₃-C₁₂ cycloalkyl, C₃-C₁₂ halocycloalkyl, C₆-C₁₀ aryl, C₇-C₁₀ aralkyl, C₆-C₁₀ aryloxy or C₇-C₁₀ aralkyloxy. In yet further embodiments, R₂ is selected from —H, halogen, cyano, nitro, azyl, lower alkyl, lower haloalkyl, lower alkoxy, lower haloalkoxy, C₃-C₁₀ cycloalkyl, C₃-C₁₀ halocycloalkyl, C₆-C₁₀ aryl, C₇-C₁₀ aralkyl, C₆-C₁₀ aryloxy or C₇-C₁₀ aralkyloxy. In even further embodiments, R₂ is selected from H, halogen, cyano, nitro, azyl; or C₁-C₄ alkyl, C₁-C₄ alkoxy, C₃-C₆ cycloalkyl, C₆-C₁₀ aryl, C₇-C₈ aralkyl, C₆-C₈ aryloxy or C₇-C₁₀ aralkyloxy, optionally substituted with halogens. In even more further embodiments, R₂ is selected from —H, —F, —Cl, —Br, cyano, nitro, azyl; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl, cyclohexyl, phenyl, indenyl, naphthyl, benzyl, phenemyl, benzyloxy, Phenylethoxy, methylbenzyloxy, ethylbenzyloxy or propylbenzyloxy, optionally substituted with halogens. In a preferred embodiment of the present invention, R₂ is selected from —H, —F, —Cl, —Br, methyl, trifluoromethyl, methoxyl.

In some embodiments, R₀ is selected from —H, —F, —Cl, —Br; or methyl, ethyl, propyl, isopropyl, n-butyl. isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl or cyclohexyl, optionally substituted with halogens. In a preferred embodiment of the present invention. R₀ is selected from —H, —F, —Cl, —Br, methyl, trifluoromethyl.

In some embodiments, R₃ is selected from —H, halogen, cyano, nitro, C₁-C₆ alkyl, C₁-C₆ haloalkyl, C₁-C₆ alkoxy, C₁-C₆ haloalkoxy, C₃-₁₂ cycloalkyl, C₃-C₁₂ halocycloalkyl, C₁₋₆ -alkylsulfonyl-C₁₋₆alkoxy, C₁₋₆alkylthio-C₁₋₆-alkoxy, C₁₋₆-alkoxy-C₁₋₆ alkoxy or 1,1-dioxidotetrahydrothiopyranyloxy; R₂ is selected from —H, halogen, cyano, nitro, azyl, lower alkyl, lower haloalkyl, lower alkoxy, lower haloalkoxy, C₃-C₁₆ cycloalkyl, C₃-C₁₆ halocycloalkyl, C₆-C₁₂ aryl, C₇-C₁₂ aralkyl, C₇-C₁₂ aryloxy or C₇-C₁₂ aralkyloxy; R₀ is selected from —H, —F, —Cl, —Br; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl or cyclohexyl, optionally substituted with halogens; R₄, R₅, R₆ and R₇ are each independently selected from —H, halogen, cyano, nitro; or hydroxyl, hydroxymethyl, hydroxyethyl, 1-hydroxyethyl, hydroxypropyl, 1-hydroxypropyl, 2-hydroxypropyl, Hydroxyisopropyl, methyl, ethyl, propyl, isopropyl, n-butyl, isobutyl, tertbutyl, methoxyl, oxethyl, isopropoxy, butoxy, isobutoxy, tert-butoxy, sec-butoxy, azyl, dimethylamino, methylthio, sulfuryl, acetyl, cyclopropvl, cyclobutyl, cyclopentyl or cyclohexyl, optionally substituted by —F, —Cl or —Br. In some further embodiments, wherein, R₃ is selected from —H, halogen, cyano, nitro, C₁-C₄ alkyl, C₁-C₄ haloalkyl, C₁-C₄ alkoxy, C₁-C₄ haloalkoxy, C₃-C₁₀ cycloalkyl, C₃-C₁₀ halocycloalkyl, C₁₋₄ alkylsulfonyl-C₁₋₄ alkoxy, C₁₋₄ -alkylthio-C₁₋₄ alkoxy, C₁₋₄ -alkoxy-C₁₋₄ alkoxy or 1,1-dioxidotetrahydrothiopyranyloxy; R₂ is selected from H, halogen, cyano, nitro, azyl, lower alkyl, lower haloalkyl, lower alkoxy, lower haloalkoxy, C₃-C₁₂ cycloalkyl, C₃-C₁₂ halocycloalkyl, C₆-C₁₀ aryl, C₇-C₁₀ aralkyl, C₆-C₁₀ aryloxy or C₇-C₁₀ aralkyloxy; R₀ is selected from —H, —F, —Cl, —Br; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl or cyclohexyl, optionally substituted with halogens; R₄, R₅, R₆ and R₇ are each independently selected from —H, halogen, cyano, nitro; or hydroxyl, hydroxymethyl, hydroxyethyl, 1-hydroxyethyl, hydroxypropyl, 1-hydroxypropyl, 2-hydroxypropyl, Hydroxyisopropyl, methyl, ethyl, propyl, isopropyl, n-butyl, isobutyl, tertbutyl, methoxyl, oxethyl, isopropoxy, butoxy, isobutoxy, tert-butoxy, sec-butoxy, azyl, dimethylamino, methylthio, sulfuryl, acetyl, cyclopropyl, cyclobutyl, cyclopentyl or cyclohexyl, optionally substituted by —F, —Cl or —Br. In yet further embodiments, R₃ is selected from —H, halogen, cyano, nitro; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl, cyclohexyl, methylsulfonylmethoxyl, ethylsulfonyloxethyl, methylsulfonylpropoxy, methylsulfonylbutoxy, ethylsulfonylmethoxyl, ethylsulfonyloxethyl: ethylsulfonylpropoxy, ethylsulfonylbutoxy, methylthiomethoxyl, methylthiooxethyl, methylthiopropoxy, methylthiobutoxy, ethylthiomethoxyl, ethylthiooxethyl, ethylthiopropoxy, ethylthiobutoxy, methoxylmethoxyl, methoxyloxethyl, methoxylpropoxy, methoxylbutoxy, oxethylmethoxyl, oxethyloxethyl, oxethylpropoxy, oxethylbutoxy, or 1,1-dioxidotetrahydrothiopyranyloxy, optionally substituted by —F, —Cl or —Br; R₂ is selected from —H, halogen, cyano, nitro, azyl; or C₁-C₄ alkyl, C₁-C₄ alkoxy, C₃-C₆ cycloalkyl, C₆-C₁₀ aryl, C₇-C₇ aralkyl, C₆-C₈ aryloxy or C₇-C₁₀ aralkyloxy, optionally substituted with halogens; R₀ is selected from —H, —F, —Cl, —Br; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl or cyclohexyl, optionally substituted with halogens; R₄, R₅, R₆ and R₇ are each independently selected from —H, halogen, cyano, nitro; or hydroxyl, hydroxymethyl, hydroxyethyl, 1-hydroxyethyl, hydroxypropyl, 1-hydroxypropyl, 2-hydroxypropyl, Hydroxyisopropyl, methyl, ethyl, propyl, isopropyl, n-butyl, isobutyl, tertbutyl, methoxyl, oxethyl, isopropoxy, butoxy, isobutoxy, tert-butoxy, sec-butoxy, azyl, dimethylamino, methylthio, sulfuryl, acetyl, cyclopropyl, cyclobutyl, cyclopentyl or cyclohexyl, optionally substituted by —F, —Cl or —Br. In even further embodiments, R₃ is selected from —H, halogen, cyano, nitro; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl, cyclohexyl, methylsulfonylmethoxyl, methylsulfonyloxethyl, methylsulfonylpropoxy, methylsulfonylbuloxy, ethylsulfonylmethoxyl, ethylsulfonyloxethyl, ethylsulfonylpropoxy, ethylsulfonylbutoxy, methylthiomethoxyl, methylthiooxethyl, methylthiopropoxy, methylthiobutoxy, ethylthiomethoxyl, ethylthiooxethyl, ethylthiopropoxy, ethylthiobutoxy, methoxylmethoxyl; methoxyloxethyl, methoxylpropoxy, methoxylbutoxy, oxethylmethoxyl, oxethyloxethyl, oxethylpropoxy, oxethylbutoxy, or 1,1-dioxidotetrahydrothiopyranyloxy, optionally substituted by —F, —Cl or —Br; R₂ is selected from —H, —F, —Cl, —Br, cyano, nitro, azyl; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl, cyclohexyl, phenyl, indenyl, naphthyl benzyl, phenemyl, benzyloxy, Phenylethoxy, methylbenzyloxy, ethylhenzyloxy or propylbenzyloxy, optionally substituted with halogens; R₀ is selected from —H, —F, —Cl, —Br; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl or cyclohexyl, optionally substituted with halogens; R₄, R₅, R₆ and R₇ are each independently selected from —H, halogen, cyano, nitro; or hydroxyl, hydroxymethyl, hydroxyethyl, 1-hydroxyethyl, hydroxypropyl, 1-hydroxypropyl, 2-hydroxypropyl, Hydroxyisopropyl, methyl, ethyl, propyl, isopropyl, n-butyl, isobutyl, tertbutyl, methoxyl, oxethyl, isopropoxy, butoxy, isobutoxy, tert-butoxy, sec-butoxy, azyl, dimethylamino, methylthio, sulfuryl, acetyl, cyclopropyl, cyclobutyl, cyclopentyl or cyclohexyl, optionally substituted by —F, —Cl or —Br. In even more further embodiments, R₃ is selected from —H, halogen, cyano, nitro; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl, cyclohexyl, methylsulfonylmethoxyl, methylsulfonyloxethyl, methylsulfonylpropoxy, methylsulfonylbutoxy, ethylsulfonylmethoxyl, ethylsulfonyloxethyl, ethylsulfonylpropoxy, ethylsulfonylbutoxy, methylthiomethoxyl, methylthiooxethyl, methylthiopropoxy, methylthiobutoxy, ethylthiomethoxyl, ethylthiooxethyl, ethylthiopropoxy, ethylthiobutoxy, methoxylmethoxyl, methoxyloxethyl, methoxylpropoxy, methoxylbutoxy, oxethylmethoxyl, oxethyloxethyl, oxethylpropoxy, oxethylbutoxy, or 1,1-dioxidotetrahydrothiopyranyloxy, optionally substituted by —F, —Cl or —Br; R₂ is selected from —H, —F, —Cl, —Br, methyl, trifluoromethyl or methoxyl; R₀ is selected from —H, —F, —Cl, —Br, methyl or trifluoromethyl; R₄ and R₅ are each independently selected from —H, —F, —Cl, —Br or methyl; R₆ and R₇ are each independently selected from —H, methyl, ethyl, trifluoromethyl, methoxyl or hydroxymethyl.

In a preferred embodiment of the present invention, n is 2, X is C, Y is S.

In a preferred embodiment of the present invention, R₃ is selected from —H, —F, —Cl, —Br, cyano, nitro, methyl, trifluoromethyl, methoxyl, trifluoromethoxy, cyclohexyl, methylsulfonylpropoxy, ethylsulfonylpropoxy, methylthiopropoxy, methoxylpropoxy or 1,1-dioxidotetrahydrothiopyranyloxy; R₂ is selected from —H, —F, —Cl, —Br, methyl, trifluoromethyl or methoxyl; R₀ is selected from —H, —F, —Cl, —Br, methyl or trifluoromethyl; R₄ and R₅ are each independently selected from —H, —F, —Cl, —Br or methyl; R₆and R₇ are each independently selected from —H, methyl, ethyl, trifluoromethyl, methoxyl or hydroxymethyl.

In a preferred embodiment of the present invention, n is 0, X is M, Y is O.

In a preferred embodiment of the present invention, n is 2, X is N, Y is O.

In a preferred embodiment of the present invention, n is 0, X is M, Y is S.

In a preferred embodiment of the present invention, n is 2, X is N, Y is S.

In a preferred embodiment of the present invention, n is 2, X is C, Y is S.

More specifically, the compounds and pharmaceutically acceptable salts or prodrugs thereof, of the current invention are most preferably selected from:

2-((S)-6-(((S)-4-(4-(3-methoxypropoxy)-2,6-dimethylphenyl)-2,3-dihydro-1H-inden-1-yl)oxy)-2,3-dihydrobenzo[b]thiophen-3-yl)acetic acid;

(S)-2-(6-((2,′,6′-dimethyl-4′-(3-(methylsulfonyl)propoxy)-[1,1′-biphenyl]-3-yl)methoxy)-2,3-dihydrofuro[3,2-c]pyridin-3-yl)acetic acid;

2-((3S)-6-((4-(2,6-dimethyl-4-(3-(methylsulfonyl)porpoxy)phenyl)-2,3-dihydro-1H-inden-1-yl)oxy)-2,3-dihydrofuro[3,2-c]pyridin-3-yl)acetic acid;

(S)-2-(6-((2′,6′-dimethyl-4′-(3-(methylthio)propoxy)-[1,1′-biphenyl]-3-yl)methoxy)-2,3-dihydrofuro[3,2-c]pyridin-3-yl)acetic acid;

2-((3S)-6-((4-(2′,6′-(3-(methylthio)propoxy)phenyl)-2,3-dihydro-1H-inden-1-yl)oxy)-2,3-dihydrofuro[3,2-c]pyridin-3-yl)acetic acid;

2-((3S)-6-((4-(4-(3-methoxypropoxy)-2,6-dimethylphenyl)-2,3-dihydro-1H-inden-1-yl)oxy)-2,3-dihydrofuro[3,2-c]pyridin-3-yl)acetic acid;

(S)-2-(6-((4′-(3-methoxypropoxy)-2′,6′-dimethyl-[1,1′-biphenyl]-3-yl)methoxy)-2,3-dihydrofuro[3,2-c]pyridin-3-yl)acetic acid;

(S)-2-(6-((4′-((1,1-dioxidotetrahydro-2H-thiopyran-4-yl)oxy)-2′,6′-dimethyl-[1,1-biphenyl]-3-yl)methoxy)-2,3-dibydrofuro[3,2-c]pyridin-3-yl)acetic acid;

2-((3S)-6-((4-(4-((1,1-dioxidotetrahydro-2H-thiopyran-4-yl)oxy)-2,6-dimethylphenyl)-2,3-dihydro-1H-inden-1-yl)oxy)-2,3-dihydrofuro[3,2-c]pyridin-3-yl)acetic acid;

2-((S)-6-(((R)-4-(4-(3-methoxypropoxy)-2,6-dimethylphenyl)-2,3-dihydro-1H-inden-1-yl)oxy)-2,3-dihydrobenzo[b]thiophen-3-yl)acetic acid;

Ethyl 2-((S)-6-(((S)-4-(4-(3-methoxypropoxy)-2,6-dimethylphenyl)-2,3-dihydro-1H-inden-1-yl)oxy)-2,3-dihydrobenzo[b]thiophen-3-yl)acetate;

2-((S)-6-(((S)-4-(2,6-dimethyl-4-(3-(methylsulfonyl)propoxy)phenyl)-2,3-dihydro-1H-inden-1-yl)oxy)-2,3-dihydrobenzo[b]thiophen-3-yl)acetic acid;

2-((S)-6-(((R)-4-(2,6-dimethyl-4-(3-(methylsulfonyl)propoxy)phenyl)-2,3-dihydro-1H-inden-1-yl)oxy)-2,3-dihydrobenzo[b]thiophen-3-yl)acetic acid;

(2-((S)-6-(((R)-4-(2,6-dimethyl-4-(3-(methylsulfonyl)propoxy)phenyl)-2,3-dihydro-1H-inden-1-yl)oxy)-2,3-dihydrobenzo[b]thiophen-3-yl)acetoxy)methyl pivalate;

(2-((S)-6-(((R)-4-(4-(3-methoxypropoxy)-2,6-dimethylphenyl)-2,3-dihydro-1H-inden-1-yl)oxy)-2,3-dihydrobenzo[b]thiophen-3-yl)acetoxy)methyl isobutyrate;

2-((S)-6-(((S)-4-(2,6-dimethyl-4-(3-(methylsulfonyl)propoxy)phenyl)-2,3-dihydro-1H-inden-1-yl)oxy)-2,3-dihydrofuro[3,2-c]pyridin-3-yl)acetic acid;

2-((S)-6-(((R)-4-(2,6-dimethyl-4-(3-(methylsulfonyl)propoxy)phenyl)-2,3-dihydro-1H-inden-1-yl)oxy)-2,3-dihydrofuro[3,2-c]pyridin-3-yl)acetic acid;

2-((3S)-6-((4-(2,6-dimethylphenyl)-2,3-dihydro-1H-inden-1-yl)oxy)-2,3-dihydrofuro[3,2-c]pyridin-3-yl)acetic acid;

(S)-2-(6-((2′,6′-dimethyl-[1,1′-biphenyl]-3-yl)methoxy)-2,3-dihydrofuro[3,2-c]pyridin-3-yl)acetic acid;

2-((3S)-6-((4-(4,6-dimethylpyrimidin-5-yl)-2,3-dihydro-1H-inden-1-yl)oxy)-2,3-diliydrofuro[3,2-c]pyridin-3-yl)acetic acid;

(S)-2-(6-((3-(4,6-dimethylpyrimidin-5-yl)benzyl)oxy)-2,3-dihydrofuro[3,2-c]pyridin-3-yl)acetic acid;

2-((3S)-6-((4-(2,4-dimethlpyridin-3-yl)-2,3-dihydro-1H-inden-1-yl)oxy)-2,3-dihydrofuro[3,2-c]pyridin-3-yl)acetic acid;

(S)-2-(6-((3-(2,4-dimethylpyridin-3-yl)benzyl)oxy)-2,3-dihydnofuro[3,2-c]pyridin-3-yl)acetic acid;

2-((3S)-6-((4-morpholino-2,3-dihydro-1H-inden-1-yl)oxy)-2,3-dihydrofuro[3,2-c]pyridin-3-yl)acetic acid;

(S)-2-(6-((3-morpholinobenzyl)oxy)-2,3-dihydrofuro[3,2-c]pyridin-3-yl)acetic acid;

2-<(3S)-6-((4-(1-methyl-1H-imidazol-2-yl)-2,3-dihydro-1H-inden-1-yl)oxy)-2,3-dihydrofuro[3,2-c]pyridin-3-yl)acetic acid;

(S)-2-(6-((3-(1-methyl-1H-imidazol-2-yl)benzyl)oxy)-2,3-dihydrofuro[3,2-c]pyridin-3-yl)acetic acid;

2-((3S)-6-((4-(2,6-dimethylphenyl)-2,3-dihydro-1H-inden-1-yl)oxy)-2,3-dihydrothieno[3,2-c]pyridin-3-yl)acetic acid;

(S)-2-(6-((2′,6′-dimethyl-[1,1′-biphenyl]-3-yl)methoxy)-2,3-dihydrothieno[3,2-c]pyridin-3-yl)acetic acid;

2-((3S)-6-((4-(4,6-dimethylpyrimidin-5-yl)-2,3-dihydro-1H-inden-1-yl(oxy)-2,3-dihydrothieno[3,2 2-c]pyridin-3-yl)acetic acid;

(S)-2-(6-((3-(4,6-dimethylpyrimidin-5-yl)benzyl)oxy)-2,3-dihydrothieno[3,2-c]pyridin-3-yl)acetic acid;

2-((3S)-6-((4-(2,4-dimethylpyridin-3-yl)-2,3-dihydro-1H-inden-1-yl)oxy)-2,3-dihydrothieno[3,2-c]pyridin-3-yl)acetic acid;

(S)-2-(6-((3-(2,4-dimethylpyridin-3-yl)benzyl)oxy)-2,3-dihydrothieno[3,2-c]pyridin-3-yl)acetic acid;

2-((3S)-6-((4-morpholino-2,3-dihydro-1H-inden-1-yl)oxy)-2,3-dihydrothieno[3,2-c]pyridin-3-yl)acetic acid;

(S)-2-(6-((3-morpholinobenzyl)oxy)-2,3-dihydrothieno[3,2-c]pyridin-3-yl)acetic acid;

2-((3S)-6-((4-(1-methyl-1H-imidazol-2-yl)-2,3-dihydro-1H-inden-1-yl)oxy)-2,3-dihydrothieno[3,2-c]pyridin-3-yl)acetic acid;

(S)-2-(6-((3-(1-methyl-1H-imidazol-2-yl)benzyl)oxy)-2,3-dihydrothieno[3,2-c]pyridin-3-yl)acetic acid;

2-((3S)-6-((4-(2,6-dimethyl-4-(3-(methylsulfonyl)propoxy)phenyl)-2,3-dihydro-1H-inden-1-yl)oxy)-2,3-dihydrothieno[3,2-c]pyridin-3-yl)acetic acid;

(S)-2-(6-((2′,6′-dimethyl-4′-(3-(methylsulfonyl)propoxy)-[1,1′-biphenyl]-3-yl)methoxy)-2,3-dihydrothieno[3,2-c]pyridin-3-yl)acetic add;

2-((3S)-6-((4-(2,6-dimethyl-4-(3-(methylthio)propoxy)phenyl)-2,3-dihydro-1H-inden-1-yl)oxy)-2,3-djhydrothieno[3,2-c]pyridin-3-yl)acetic add;

(S)-2-(6-((2′,6′-dimethyl-4′-(3-(methylthio)propoxy)-[1,1′-biphenyl]-3-yl)methoxy)-2,3-dihydrothieno[3,2-c]pyridin-3-yl)acetic acid;

2-((3S)-6-((4-(4-(3-methoxypropoxy)-2,6-dimethylphenyl)-2,3-dihydro-1H-inden-1-yl)oxy)-2,3-dihydrothieno[3,2-c]pyridin-3-yl)acetic acid;

(S)-2-(6-((4′-(3-methoxypropoxy)-2′,6′-dimethyl-[1,1′-biphenyl]-3-yl)methoxy)-2,3-dihydrothieno[3,2-c]pyridin-3-yl)acetic acid;

2-((3S)-6-((4-(4-((1,1-dioxidotetrahydro-2H-thiopyran-4-yl)oxy)-2,6-dimethylphenyl)-2,3-dihydro-1H-inden-1-yl)oxy)-2,3-dihydrothieno[3,2-c]pyridin-3-yl)acetic acid;

(S)-2-(6-((4′-((1,1-dioxidotetrahydro-2H-thiopyran-4-yl)oxy)-2′,6′-dimethyl-[1,1′-biphenyl]-3-yl)methoxy)-2,3-dihydrothieno[3,2-c]pyridin-3-yl)acetic acid;

2-((3S)-6-((4-(2,6-dimethylphenyl)-2,3-dihydro-1H-inden-1-yl)oxy)-2,3-dihydrobenzo[b]thiophen-3-yl)acetic acid;

2-((3S)-6-((4-(4,6-dimethy)pyrimidin-5-yl)-2,3-dihydro-1H-inden-1-yl)oxy)-2,3-dihydrobenzo[b]thiophen-3-yl)acetic acid;

2-((3S)-6-((4-(2,4-dimethylpyridin-3-yl)-2,3-dihydro-1H-inden-1-yl)oxy)-2,3-dihydrobenzo[b]thiophen-3-yl)acetic acid;

2-((3S)-6-((4-morpholino-2,3-dihydro-1H-inden-1-yl)oxy)-2,3-dihydrobenzo[b]thiophen-3-yl)acetic acid;

2-((3S)-6-((4-(1-methyl-1H-imidazof-2-yl)-2,3-dihydro-1H-inden-1-yl)oxy)-2,3-dihydrobenzo[b]th iophen-3-yl)acetic acid;

2-((3S)-S-((4-(2,6-dimethyl-4-(3-(methylsulfonyl)propoxy)pheny)-2,3-dihydro-1H-inden-1-yl)oxy)-2,3-dihydrobenzo[b]thiophen-3-yl)acetic acid;

2-((3S)-S-((4-(2,6-dimethyl-4-(3-(methylsulfonyl)propoxy)pheny)-2,3-dihydro-1H-inden-1-yl)oxy)-2,3-dihydrobenzo[b]thiophen-3-yl)aoetic acid;

2-((3S)-6-((4-(4-(3-methoxypropoxy)-2,6-dimethylphenyl)-2,3-dihydro-1H-inden-1-yl)oxy)-2,3-dihydrobenzo[b]thiophen-3-yl)acetic acid;

2-((3S)-6-((4-(4-((1,1-dioxidotetrahydro-2H-thiopyran-4-yl)oxy)-2,6-dimethylphenyl)-2,3-dihydro-1H-inden-1-yl)oxy)-2,3-dihydrobenzo[b]thiophen-3-yl)acetic acid;

2-((3S)-6-((4-mesityl-2,3-dihydro-1H-inden-1-yl)oxy)-7-methyl-2,3-dihydrofuro[3,2-c]pyridin-3-yl)acetic acid;

2-((3S)-6-((4-(4-methoxy-2,6-dimethylphenyl)-2,3-dihydro-1H-inden-1-yl)oxy)-2,3-dihydrofuro[3,2-c]pyridin-3-yl)acetic acid;

2-((3S)-7-bromo-6-((4-(2,6-dimethyl-4-(trifluoromethy))phenyl)-2,3-dihydro-1H-inden-1-yl)oxy)-2,3-dihydrofuro[3,2-c]pyridin-3-yl)acetic acid;

2-((3S)-6-((4-(2,6-dimethyl-4-(trifluoromethoxy)phenyl)-2,3-dihydro-1H-inden-1-yl)oxy)-2,3-dihydrofuro[3,2-c]pyridin-3-yl)acetic acid;

2-((3S)-6-((4-(4-cyclohexyl-2,6-diniethylpbenyl)-6-methyl-2,3-dihydro-1H-inden-1-yl)oxy)-2,3-dihydrofuro[3,2-c]pyridin-3-yl)acetic acid;

2-((3S)-6-((4-(2,6-dimethyl-4-(3-(methylsulfonyl)propoxy)pbenyl)-5-fluoro-2,3-dihydro-1H-inden-1-yl)oxy)-2,3-dihydrofuro[3,2-c]pyridin-3-yl)acetic acid;

2-((3S)-6-((4-(2,6-dimethyl-4-(3-(methylsulfonyl)propoxy)phenyl)-5-methoxy-2,3-dihydro-1H-inden-1-yl)oxy)-2,3-dihydrofuro[3,2-c]pyridin-3-yl)acetic acid;

2-((3S)-6-((4-(2,6-dimethyl-4-(3-(methylsulfonyl)propoxy)phenyl)-7-(trifluoromethy)-2,3-dihydro-1H-inden-1-yl)oxy)-2,3-dihydrofuro[3,2-c]pyridin-3yl)-acetic acid;

2-((3S)-6-((4-(3,5-dichloro-2,6-dimethyl-4-(3-(methylsulfonyl)propoxy)phenyl)-2,3-dihydro-1H-inden-1-yl)oxy)-2,3-dihydrofuro[3,2-c]pyridin-3-yl)acetic acid;

2-((3R)-6-((4-(3-fluoro-2,6-dimethyl-4-(3-2,3-dihydro-1H-inden-1-yl)oxy)-2,3-dihydrofuro[3,2-c]pyridin-3-yl)acetic acid;

2-((3R)-6-((4-(2-methyl-4-(3-(methylsulfonyl)propoxy)phenyl)2,3-dihydro-1H-inden-1-yl)oxy)-2,3-dihydrofuro[3,2-c]pyridin-3-yl)acetic acid;

2-((3S)-6-((4-(4-cyano-2,6-dimethylphenyl)-5,7-dimethyl-2,3-dihydro-1H-inden-1-yl)oxy)-2,3-dihydrofuro[3,2-c]pyridin-3-yl)acetic acid;

2-((3S)-6-((4-(2,6-dimethyl-4-nitrophenyl)-2,3-dihydro-1H-inden-1-yl)oxy)-2,3-dihydrofuro[3,2-c]pyridin-3-yl)acetic acid;

2-((3R)-6-((4-(4-bromo-2,6-dimethylphenyl)-2,3-dihydro-1H-inden-1-yl)oxy)-2,3-dihydrofuro[3,2-c]pyridin-3-yl)acetic acid;

2-((3S)-6-((4-(2,3,5,6-tetramethyl-4-(3-(methylsulfonyl)propoxy)phenyl)-2,3-dihydro-1H-inden-1-yl)oxy)-2,3-dihydrofuro[3,2-c]pyridin-3-yl)acetic acid;

2-((3S)-6-((4-(2-(hydroxymethyl)-6-methyl-4-(3-(methylsulfonyl)propoxy)phenyl)-2,3-dihydro-1H-inden-1-yl)oxy)-2,3-dihydrofuro[3,2-c]pyridin-3-yl)acetic acid;

2-((3S)-6-((4-(2-methoxy-6-methyl-4-(3-(methylsulfonyl)propoxy)phenyl)-2,3-dihydro-1H-inden-1-yl)oxy)-2,3-dihydrofuro[3,2-c]pyridin-3-yl)acetic acid;

2-((3S)-6-((4-(4-(3-(methylsulfonyl)propoxy)-2,6-bis(trifluoromethyl)phenyl)-2,3-dihydro-1H-inden-1-yl)oxy)-2,3-dihydrofuro[3,2-c]pyridin-3-yl)acetic acid;

2-((3R)-6-((4-(2,6-dimethyl-4-(3-(methylsulfonyl)propoxy)phenyl)-2,3-dihydro-1H-inden-1-yl)oxy)-7-(trifluoromethyl)-2,3-dihydrofuro[3,2-c]pyrdin-3-yl)acetic acid;

2-((3R)-6-((4-(4-(3-(ethylsulfonyl)propoxy)-2,6-dimethylphenyl)-2,3-dihydro-1H-inden-1-yl)oxy)-2,3-dihydrofuro[3,2-c]pyridin-3-yl)acetic acid;

2-((3R)-6-((4-(2,6-diethyl-4-(3-(methylsulfonyl)propoxy)phenyl)-2,3-dihydro-1H-inden-1-yl)oxy)-2,3-dihydrofuro[3,2-c]pyridin-3-yl)acetic acid;

2-((3R)-6-((4-(4-(3-(methylsulfonyl)propoxy)phenyl)-2,3-dihydro-1H-inden-1-yl)oxy)-2,3-dihydrofuro[3,2-c]pyridin-3-yl)acetic acid;

(S)-2-(7-methyl-6-((2′,4′,6′-trimethyl-[1′,1′-biphenyl]-3-yl)methoxy)-2,3-dihydrofuro[3,2-c]pyridin-3-yl)acetic acid;

(S)-2-(6-((4′-methoxy-2′,6′-dimethyl-[1,1′-biphenyl]-3-yl)methoxy)-2,3-dihydrofuro[3,2-c]pyridin-3-yl)acetic acid;

(S)-2-(7-bromo-6-((2′,6′-dimethyl-4′-(trifluoromethyl)-[1,1′-biphenyl]-3-yl)methoxy)-2,3-dihydrofuro[3,2-c]pyridin-3-yl)acetic acid;

(S)-2-(6-((2′,6′-dimethyl-4′-(trifluoromethoxy)-[1,1′-biphenyl]-3-yl)methoxy)-2,3-dihydrofuro[3,2-c]pyridin-3-yl)acetic acid;

(S)-2-(6-((4′-cyclohexyl-2′,5,6′-trimethyl -[1,1′-biphenyl]-3-yl)methoxy)-2,3-dihydrofuro[3,2-c]pyridin-3-yl)acetic acid;

(S)-2-(6-((6-fluoro-2′,6′-dimethyl-4′-(3-(methylsulfonyl)propoxy)-[1,1′-biphenyl]-3-yl)methoxy)-2,3-dihydrofuro[3,2-c]pyridin-3-yl)acetic acid;

(S)-2-(6-((6-methoxy-2′,6′-dimelhyl-4′-(3-(methylsulfonyl)propoxy)-[1,1′-biphenyl]-3-yl)methoxy)-2,3-dihydrofuro[3,2-c]pyridin-3-yl)acetic acid;

(S)-2-(6-((2′,6′-dimethyl-4′-(3-(methylsulfonyl)propoxy)-4-(trifluoromethyl)-[1,1′-biphenyl]-3-yl)methoxy)-2,3-dihydrofuro[3,2-c]pyridin-3-yl)acetic acid;

(S)-2-(6-((3′,5′-dichloro-2′,6′-dimethyl-4′-(3-(methylsulfonyl)propoxy)-[1,1′-biphenyl]-3-yl)methoxy)-2,3-dihydrofuro[3,2-c]pyridin-3-yl)acetic acid;

(S)-2-(6-((3′-fluoro-2′,6′-dimethyl-4′-(3-(methylsulfonyl)propoxy)-[1,1′-biphenyl]-3-yl)methoxy)-2,3-dihydrofuro[3,2-c]pyridin-3-yl)acetic acid;

(S)-2-(6-((2′-methyl-4′-(3-(methylsulfonyl)propoxy)-[1,1′-biphenyl]-3-yl)methoxy)-2,3-dihydrofuro[3,2-c]pyridin-3-yl)acetic acid;

(S)-2-(6-((4′-cyano-2′,4, 6,6′-tetramethyl-[1,1′-biphenyl]-3-yl)methoxy)-2,3-dihydrofuro[3,2-c]pyridin-3-yl)acetic acid;

(S)-2-(6-((2′,6′-dimethyl-4′-nitro-[1,1′-biphenyl]-3-yl)methoxy)-2,3-dihydrofuro[3,2-c]pyridin-3-yl)acetic acid;

(S)-2-(6-((4′-bromo-2′,6′-dimethyl-[1,1′-biphenyl]-3-yl)methoxy)-2,3-dihydrofuro[3,2-c]pyridin-3-yl)acetic acid;

(S)-2-(6-((2′,3′,5′,6′-tetramethyl-4′-(3-(methylsulfonyl)propoxy)-[1,1′-biphenyl]-3-yl)methoxy)-2,3-dihydrofu o[3,2-c]pyridin-3-yl)acetic acid;

(S)-2-(6-((2′-(hydroxymethyl)-6′-methyl-4′-(3-(methylsulfonyl)propoxy)-[1,1′-biphenyl]-3-yl)methoxy)-2,3-dihydrofuro[3,2-c]pyridin-3-yl)acetic acid;

(S)-2-(6-((2′-methoxy-6′-methyl-4′-(3-(methylsulfonyl)propoxy)-[1,1′-biphenyl]-3-yl)methoxy)-2,3-dihydrofuro[3,2-c]pyridin-3-yl)acetic acid;

(S)-2-(6-((4′-(3-(methylsylfonyl)propoxy)-2′,6′-bis(trifluoromethyl)-[1,1′-biphenyl]-3-yl)methoxy)-2,3-dihydrofuro[3,2-c]pyridin-3-yl)acetic acid;

(S)-2-(6-((2′,6′-dimethyl-4′-(3-(methylsulfonyl)propoxy)-[1,1′-biphenyl]-3-yl)methoxy)-7-(trifluoromethyl)-2,3-dihydrofuro[3,2-c]pyridin-3-yl)acetic acid;

(S)-2-(6-((4′-(3-(ethylsulfonyl)propoxy)-2′,6′-dimethyl-[1,1′-biphenyl]-3-yl)methoxy)-2,3-dihydrofuro[3,2-c]pyridin-3-yl)acetic acid;

(S)-2-(6-((2′,6′-diethyl-4′-(3-(methylsulfonyl)propoxy)-[1,1′-biphenyl]-3-yl)methoxy)-2,3-dihydrofuro[3,2-c]pyridin-3-yl)acetic acid;

(S)-2-(6-((4′-(3-(methylsulfonyl)propoxy)-[1,1′-biphenyl]-3-yl)methoxy-2,3-dihydrofuro[3,2-c]pyridin-3-yl)acetic acid;

2-((3S)-6-((4-mesityl-2,3-dihydro-1H-inden-1-yl)oxy)-7-methyl-2,3-dihydrothieno[3,2-c]pyridin-3-yl)acetic acid;

2-((3S)-6-((4-(4-methoxy-2,6-dimethylphenyl)-2,3-dihydro-1H-inden-1-yl)oxy)-2,3-dihydrothieno[3,2-c]pyridin-3-yl)acetic acid;

2-((3S)-7-bromo-6-((4-(2,6-dimethyl-4-(trifluoromethyl)phenyl)-2,3-dihydro-1H-inden-1-yl)oxy)-2,3-dihydrothieno[3,2-c]pyridin-3-yl)acetic acid;

2-((3S)-6-((4-(2,6-dimethyl -4-(trifluoromethoxy)phenyl)-2,3-dihydro-1H-inden-1-yl)oxy)-2,3-dihydrothieno[3,2-c]pyridin-3-yl)acetic acid;

2-((3S)-6-((4-(4-cyclohexyl-2,6-dimethylphenyl)-6-methyl-2,3-dihydro-1H-inden-1-yl)oxy)-2,3-dihydrothieno[3,2-c]pyridin-3-yl)acetic acid;

2-((3S)-6-((4-(2,6-dimethyl-4-(3-(methylsulfonyl)propoxy)phenyl)-5-fluoro-2,3-dihydro-1H-inden-1-yl)oxy)-2,3-dihydrothieno[3,2-c]pyridin-3-yl)acetic acid;

2-((3S)-6-((4-(2,6-dimethyl-4-(3-(methylsulfonyl)propoxy)phenyl)-5-methoxy-2,3-dihydro-1H-inden-1-yl)oxy)-2,3-dihydrothieno[3,2-c]pyridin-3-yl)acetic acid;

2-((3S)-6-((4-(2,6-dimethyl-4-(3-(methylsulfonyl)propoxy)pbenyl)-7-(trifluoromethyl)-2,3-dihydro-1H-inden-1-yl)oxy)-2,3-dibydrothieno[3,2-c]pyridin-3-yl)acetic acid;

2-((3S)-6-((4-(3,5-dichloro-2,6-dimethyl-4-(3-(methylsulfonyl)propoxy)phenyl)-2,3-dihydro-1H-inden-1-yl)oxy)-2,3-dihydrothieno[3,2-c]pyridin-3-yl)acetic acid;

2-((3S)-6-((4-(3-fluro-2,6-dimethyl-4-(3-(methylsulfonyl)propoxy)phenyl)-2,3-dihydro-1H-inden-1-yl)oxy)-2,3-dihydrothieno[3,2-c]pyridin-3-yl)acetic acid;

2-((3S)-6-((4-(2-methyl-4-(3-(methylsulfonyl)propoxy)phenyl)-2,3-dihydro-1H-inden-1-yl)oxy)-2,3-dihydrothieno[3,2-c]pyridin-3-yl)acetic add;

2-((3S)-6-((4-(4-cyano-2,6-dimethylphenyl)-5,7-dimethyl-2,3-dihydro-1H-inden-1-yl)oxy)-2,3-dihydrothieno[3,2-c]pyridin-3-yl)acetic acid;

2-((3S)-6-((4-(2,6-dimethyl-4-nitrophenyl)-2,3-dihydro-1H-inden-1-yl)oxy)-2,3-dihydrothieno[3,2-c]pyridin-3-yl)acetic acid;

2-((3S)-6-((4-(4-bromo-2,6-dimethylphenyl)-2,3-dihydro-1H-inden-1-yl)oxy)-2,3-dihydrothieno[3,2-c]pyridin-3-yl)acetic acid;

2-((3S)-6-((4-(2,3,5,6-tetramethyl-4-(3-(methylsulfonl)propoxy)phenyl)-2,3-dihydro-1H-inden-1-yl)oxy)-2,3-dihydrothieno[3,2-c]pyridin-3-yl)acetic acid;

2-((3S)-6-((4-(2-(hydroxymethyl)-6-methyl-4-(3-(methylsulfonyl)propoxy)phenyl)-2,3-dihydro-1H-inden-1-yl)oxy)-2,3-dihydrothieno[3,2-c]pyridin-3-yl)acetic acid;

2-((3S)-6-((4-(2-methoxy-6-methyl-4-(3-(methylsulfonyl)propoxy)phenyl)-2,3-dihydro-1H-inden-1-yl)oxy)-2,3-dihydrothieno[3,2-c]pyridin-3-yl)acetic acid;

2-((3S)-6-((4-(4-(3-(methylsulfonyl)propoxy)-2,6-bis(trifluoromethyl)phenyl)-2,3-dihydro-1H-inden-1-yl)oxy)-2,3-dihydrothieno[3,2-c]pyridin-3-yl)acetic acid;

2-((3S)-6-((4-(2,6-dimethyl-4-(3-(methylsulfonyl)propoxy)phenyl)-2,3-dihydro-1H-inden-1-yl)oxy)-7-(trifluoromethyl)-2,3-dihydrothieno[3,2-c]pyridin-3-yl)acetic acid;

2-((3S)-6-((4-(4-(3-(ethylsulfonyl)propoxy)-2,6-dimethylphenyl)-2,3-dihydro-1H-inden-1-yl)oxy)-2,3-dihydrothieno[3,2-c]pyridin-3-yl)acetic acid;

2-((3S)-6-((4-(2,6-diethyl-4-(3-(methylsulfonyl)propoxy)phenyl)-2,3-dihydro-1H-inden-1-yl)oxy)-2,3-dihydrothieno[3,2-c]pyridin-3-yl) acetic acid;

2-((3S)-6-((4-(4-(3-(methylsulfonyl)propoxy)phenyl)-2,3-dihydro-1H-inden-1-yl)oxy)-2,3-dihydrothieno[3,2-c]pyridin-3-yl) acetic acid;

(S)-2-(7-methyl-S-((2′,4′,6′-trimethyl-[1,1′-biphenyl]-3-yl)methoxy)-2,3-dihydrothieno[3,2-c]pyridin-3-yl)acetic acid;

(S)-2-(6-((4′-methoxy-2′,6′-dimethyl-[1,1′-biphenyl]-3-yl)methoxy)-2,3-dihydrothieno[3,2-c]pyridin-3-yl)acetic acid;

(S)-2-(7-bromo-6-((2′,6′-dimethyl-4′-(trifluoromethyl)-[1,1′-biphenyl]-3-yl)methoxy)-2,3-dihydrothieno[3,2-c]pyridin-3-yl)acetic acid;

(S)-2-(6-((2′,6′-dimethyl-4′-(trifluoromethoxy)-[1,1′-biphenyl]-3-yl)methoxy)-2,3-dihydrothieno[3,2-c]pyridin-3-yl)acetic acid;

(S)-2-(6-((4′-cyclohexyl-2′,5,6′-trimethyl-[1,1′-biphenyl]-3-yl)methoxy)-2,3-dihydrothieno[3,2-c]pyridin-3-yl)acetic acid;

(S)-2-(6-((6-fluoro-2′,6′-dimethyl-4′-(3-(methylsulfonyl)propoxy)-[1,1′-biphenyl]-3-yl)methoxy)-2,3-dihydrothieno[3,2-c]pyridin-3-yl)acetic acid;

(S)-2-(6-((6-methoxy-2′,6′-dsmethyl-4′-(3-(methylsulfonyl)propoxy)-[1,1′-biphenyl]-3-yl)methoxy)-2,3-dihydrothieno[3,2-c]pyridin-3-yl)acetic acid;

(S)-2-(6-((2′,6′-dimethyl-4′-(3-(methylsulfonyl)propoxy)-4-(trifluoromethyl)-[1,1′-biphenyl]-3-yl)methoxy)-2,3-dihydrothieno[3,2-c]pyridin-3-yl)acetic acid;

(S)-2-(6-((3′,5′-dichloro-2′,6′-dimethyl-4′-(3-(methylsulfonyl)propoxy)-[1,1′-biphenyl]-3-yl)methoxy)-2,3-dihydrothieno[3,2-c]pyridin-3-yl)acetic acid;

(S)-2-(6-((3′-fluoro-2′,6′-dimethyl-4′-(3-(methylsulfonyl)propoxy)-[1,1′-biphenyl]-3-yl)methoxy)-2,3-dihydrothieno[3,2-c]pyridin-3-yl)acetic acid;

(S)-2-(6-((2′-methyl-4′-(3-(methylsulfonyl)propoxy)-[1,1′-biphenyl]-3-yl)methoxy)-2,3-dihydrothieno[3,2-c]pyridin-3-yl)acetic acid;

(S)-2-(6-((4′-cyano-2′,4,6,6′-tetramethyl-[1,1′-biphenyl]-3-yl)methoxy )-2,3-dihydrothieno[3,2-c]pyridin-3-yl)acetic acid;

(S)-2-(6-((2′,6′-dimethyl-4′-nitro-[1,1′-biphenyl]-3-yl)methoxy)-2,3-dihydrothieno[3,2-c]pyridin-3-yl)acetic acid;

(S)-2-(6-((4′-bromo-2′,6′-dimethyl-[1,1′-biphenyl]-3-yl)methoxy)-2,3-dihydrothieno[3,2-c]pyridin-3-yl)acetic acid;

(S)-2-(6-((2′,3′,5′,6′-tetramethyl-4′-(3-(melhylsulfonyl)propoxy)-[1,1′-biphenyl]-3-yl)methoxy)-2,3-dihydrothieno[3,2-c]pyridin-3-yl)acetic acid;

(S)-2-(6-((2′-(hydroxymethyl)-6′-methyl-4′-(3-(methylsulfonyl)propoxy)-[1,1′-biphenyl]-3-yl)methoxy)-2,3-dihydrothieno[3,2-c]pyridin-3-yl)acetic acid;

(S)-2-(6-((2′-methoxy-6′-methyl-4′-(3-(methylsulfonyl)propoxy)-[1,1′-biphenyl]-3-yl)methoxy)-2,3-dihydrothieno[3,2-c]pyridin-3-yl)acetic acid;

(S)-2-(6-((4′-(3-(methylsylfonyl)propoxy)-2′,6′-bis(trifluoromethyl)-[1,1′-biphenyl]-3-yl)methoxy)-2,3-dihydrothieno[3,2-c]pyridin-3-yl)acetic acid;

(S)-2-(6-((2′,6′-diethyl-4′-(3-(methylsulfonyl)propoxy)-[1,1′-biphenyl]-3-yl)methoxy)-7-(trifluoromethyl)-2,3-dihydrothieno[3,2-c]pyridin-3-yl)acetic acid;

(S)-2-(6-((4′-(3-(ethylsulfonyl)propoxy)-2′,6′-dimethyl-[1,1′-biphenyl]-3-yl)methoxy)-2,3-dihydrothieno[3,2-c]pyridin-3-yl)acetic acid;

(S)-2-(6-((2′,6′-diethyl-4′-(3-(methylsulfonyl)propoxy)-[1,1′-biphenyl]-3-yl)methoxy)-2,3-dihydrothieno[3,2-c]pyridin-3-yl)acetic acid;

(S)-2-(6-((4′-(3-(methylsulfonyl)propoxy)-[1,1′-biphenyl]-3-yl)methoxy)-2,3-dihydrothieno[3,2-c]pyridin-3-yl)acetic acid;

2-((3S)-6-((4-mesityl-2,3-dihydro-1H-inden-1-yl)oxy)-7-methyl-2,3-dihydrobenzo[b]thiophen-3-yl)acetic acid;

2-((3S)-6-((4-(4-methoxy-2,6-dimethylphenyl)-2,3-dihydro-1H-inden-1-yl)oxy)-2,3-dihydrobenzo[b]thiophen-3-yl)acetic acid;

2-((3S)-7-bromo-6-((4-(2,6-dimethyl-4-(trifluoromethyl)phenyl)-2,3-dihydro-1H-inden-1-yl)oxy)-2,3-dihydrobenzo[b]thiophen-3-yl)acetic acid;

2-((3S)-6-((4-(2,6-dimethyl-4-(trifluoromethoxy)phenyl)-2,3-dihydro-1H-inden-1-yl)oxy)-2,3-dihydrobenzo[b]thiophen-3-yl)acetic acid;

2-((3S)-6-((4-(4-cyclohexyl-2,6-dimethylphenyl)-6-methyl-2,3-dihydro-1H-inden-1-yl)oxy)-2,3-dihydrobenzo[b]thiophen-3-yl)acetic acid;

2-((3S)-6-((4-(2,6-dimethyl-4-(3-(methylsulfonyl)propoxy)phenyl)-5-fluoro-2,3-dihydro-1H-inden-1-yl)oxy)-2,3-dihydrobenzo[b]thiophen-3-yl)acetic acid;

2-((3S)-6-((4-(2,6-dimethyl-4-(3-(methylsulfonyl)propoxy)phenyl)-5-methoxy-2,3-dihydro-1H-inden-1-yl)oxy)-2,3-dihydrobenzo[b]thiophen-3-yl)acetic acid;

2-((3S)-6-((4-(2,6-dimethyl-4-(3-(methylsulfonyl)propoxy)pbenyl)-7-(trifluoromethyl)-2,3-dihydro-1H-inden-1-yl)oxy)-2,3-dihydrobenzo[b]thiophen-3-yl)acetic acid;

2-((3S)-6-((4-(3,5-dichloro-2,6-dimethyl-4-(3-(methylsulfonyl)propoxy)phenyl)-2,3-dihydro-1H-inden-1-yl)oxy)-2,3-dihydrobenzo[b]thiophen-3-yl)acetic acid;

2-((3S)-6-((4-(3-fluoro-2,6-dimethyl-4-(3-(methyleulfonyl)propoxy)phenyl)-2,3-dihydro-1H-inden-1-yl)oxy)-2,3-dihydrobenzo[b]thiophen-3-y)acetic acid;

2-((3S)-6-((4-(2-methyl-4-(3-(methylsulfonyl)propoxy)pbenyl)-2,3-dihydro-1H-inden-1-yl)oxy)-2,3-dihydrobenzo[b]thiophen-3-yl)acetic acid;

2-((3S)-6-4-(4-cyano-2,6-dimethylphenyl)-5,7-dimethyl-2,3-dihydro-1H-inden-1-yl)oxy)-2,3-dihydrobenzof[b]thiophen-3-yl)acetic acid;

2-((3S)-6-((4-(2,6-dimethyl-4-nitrophenyl)-2,3-dihydro-1H-inden-1-yl)oxy)-2,6-dihydrobenzo[b]thiophen-3-yl)acetic acid;

2-((3S)-6-((4-(4-bromo-2,6-dimethylpheny)-2,3-dihydro-1H-inden-1-yl)oxy)-2,3-dihydrobenzo[b]thiophen-3-yl)acetic acid;

2-((3S)-6-((4-(2,3,5,6-tetramethyl-4-(3-(methylsulfonyl)propoxy)phenyl)-2,3-dihydro-1H-inden-1-yl)oxy)-2,3-dihydrobenzo[b]thiophen-3-yl)acetic acid;

2-((3S)-6-((4-(2-(hydroxymethyl)-6-methyl-4-(3-(methylsulfonyl)propoxy)phenyl)-2,3-dihydro-1H-inden-1-yl)oxy)-2,3-dihydrobenzo[b]thiophen-3-yl)acetic acid;

2-((3S)-6-((4-(2-methoxy-6-methyl-4-(3-(methylsulfonyl)propoxy)phenyl)-2,3-dihydro-1H-inden-1-yl)oxy)-2,3-dihydrobenzo[b]thiophen-3-yl)acetic acid;

2-((3S)-6-((4-(4-(3-(methylsulfonyl)propoxy)-2,6-bis(trifluoromethyl)phenyl)-2,3-dihydro-1H-inden-1-yl)oxy)-2,3-dihydrobenzo[b]thiophen-3-yl)acetic acid;

2-((3S)-6-((4-(2,6-dimethyl-4-(3-(methylsulfonyl)propoxy)phenyl)-2,3-dihydro-1H-inden-1-yl)oxy)-7-(trifluorometiyl)-2,3-dihydrobenzo[b]thiophen-3-yl)acetic acid;

2-((S)-6-(((R)-5-(2,6-dimethyl-4-(3-(methylsulfonyl)propoxy)phenyl)-1,2,3,4-tetrahydronaphthalen-1-yl)oxy)-2,3-dihydrobenzo[b]thiophen-3-yl)acetic acid;

2-((S)-6-(((S)-1-(4-(3-methoxypropoxy)-2,6-dimethylphenyl)-6,7,8,9-tetrahydro-5H-benzo[7]annulen-5-yl)oxy)-2,3-dihydrobenzo[b]thiophen-3-yl)acetic acid;

2-((3S)-6-((4-(4-(3-(ethylsulfonyl)propoxy)-2,6-dimethylphenyl)-2,3-dihydro-1H-inden-1-yl)oxy)-2,3-dihydrobenzo[b]thiophen-3-yl)acetic acid;

2-((3S)-6-((4-(2,6-diethyl-4-(3-(methylsulfonyl)propoxy)phenyl)-2,3-dihydro-1H-inden-1-yl)oxy)-2,3-dihydrobenzo[b]thiophen-3-yl)acetic acid; or

2-((3S)-6-((4-(4-(3-(methylsulfonyl)porpoxy)phenyl)-2,3-dihydro-1H-inden-1-yl)oxy)-2,3-dibydrobenzo[b]thiopben-3-yl)acetic acid.

The present invention also provides a method of modulating GPR₄₀ receptor function in animals or humans, by administering to the subject a therapeutically effective amount of the compound of Formular (I), or at least one pharmaceutically acceptable salt or prodrug thereof.

The present invention further provides a method for the treatment and/or prevention of type II diabetes in animals or humans, by administering to the subject a therapeutically effective amount of the compound of Formula (I), or at least one pharmaceutically acceptable salt or prodrug thereof.

The present invention further provides a method for the treatment and/or prevention of obesity in animals or humans, by administering to the subject a therapeutically effective amount of the compound of Formula (I), or at least one pharmaceutically acceptable salt or prodrug thereof.

The present invention also provides the use of the compound of Formula (I) or at least one pharmaceutically acceptable salt or prodrug thereof of the present invention in manufacturing a medicament.

The present invention also provides the use of the compound of Formula (I) or at least one pharmaceutically acceptable salt or prodrug thereof of the present invention in manufacturing a medicament. Paticularly, the present invention provides the use of the compounds of Formular (I) in manufacturing a medicament for modulating GPR₄₀ receptor function in animals or humans.

The present invention further provides the use of the compound of Formula (I) or at least one pharmaceutically acceptable salt or prodrug thereof in manufacturing a medicament for the treatment and/or prevention of type it diabetes in animals or humans.

The present invention further provides the use of the compound of Formula (I) or at least one pharmaceutically acceptable salt or prodrug thereof in manufacturing a medicament for the treatment and/or prevention of obesity in animals or humans.

The present invention also furtuer provides a pharmaceutical composition comprising a therapeutically effective amount of one or more of compounds of Forumuiar (I), or at least one pharmaceutically acceptable salt or prodrug thereof, and a pharmaceutically acceptable adjuvant.

The present invention also provides a method of modulating GPR₄₀ receptor function in animals or humans, by administering to the subject a therapeutically effective amount of at least one pharmaceutical composition described herein.

The present invention also provides a method for the treatment and/or prevention of type II diabetes in animals or humans, by administering to the subject a therapeutically effective amount of at least one pharmaceutical composition described herein.

The present invention also provides a method for the treatment and/or prevention of obesity in animals or humans, by administering to the subject a therapeutically effective amount of at least one pharmaceutical composition described herein.

Further provided is the use of a pharmaceutical composition described herein in manufacturing a medicament.

The present invention further provides the use of the pharmaceutical composition in manufacturing a medicament. Particularly, the present invention further provides the use of the pharmaceutical composition in manufacturing a medicament for modulating GPR₄₀ receptor function in animals or humans.

The present invention further provides the use of the pharmaceutical composition in manufacturing a medicament for the treatment and/or prevention of type II diabetes in animals or humans.

The present invention further provides the use of the pharmaceutical composition in manufacturing a medicament for the treatment and/or prevention of obesity in animals or humans.

“Cₚ-C_{q}” or “C_{p-q}” (where p and q are integers) refers to a radical inclusively containing from p number of carbon atoms to q number of carbon atoms. For example, C₁₋₃ denotes a radical containing 1, 2 or 3 carbon atoms.

The term “Alkyl” refers to a saturated, branched or straight-chain monovalent hydrocarbon group derived by the removal of one hydrogen atom from a single carbon atom of a parent alkane. Typical alkyl groups include, but are not limited to, methyl, ethyl, propyls such as propan-1-yl, and propan-2-yl, butyls such as butan-1-yl, butan-2-yl, 2-methyl-propan-1-yl, 2-methyl-propan-2-yl, tert-butyl, and the like. In certain embodiments, an alkyl group comprises from 1 to 20 carbon atoms. As used herein the term “lower alkyl” refers to an alkyl group comprising from 1 to 6 carbon atoms. Typical lower alkyl groups include, but are not limited to, methyl, ethyl, propyl, isopropyl, n-butyl, isobutyl, tert-butyl, s-butyl, pentyl, neopentyl or hexyl.

“Aryl” refers to a monovalent aromatic hydrocarbon group derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. For example, aryl includes 5- and 6-membered carbocyclic aromatic rings fused to a 5- to 7-membered heterocycloalkyl ring containing 1 or more heteroatoms selected from N, O, and S. In certain embodiments, an aryl group can comprise from 6 to 10 carbon atoms. In a preferred embodiment of the present invention, the C₆-C₁₀ aryl group is phenyl or naphthyl, and most preferably a phenyl group.

“Heteroaryl” refers to a monovalent heteroaromatic group derived by the removal of one hydrogen atom from a single atom of a parent heteroaromatic ring system. Heteroaryl encompasses: 5- to 7-membered aromatic, monocyclic rings containing one or more, for example, from 1 to 4, or in certain embodiments, from 1 to 3, heteroatoms selected from N, O, and S, with the remaining ring atoms being carbon; and polycyclic heterocycloalkyl rings containing one or more, for example, from 1 to 4, or in certain embodiments, from 1 to 3, heteroatoms selected from N, O, and S, with the remaining ring atoms being carbon and wherein at least one heteroatom is present in an aromatic ring. Particularly preferred heteroaryl groups are C₃-C₁₀ heteroaryl, include but are not limited to, pyrrolyl, furanyl, thienyl, pyridinyl, pyranyl, pyrazolyl, pyrimidinyl, imidazolyl, thiazolyl, oxazolyl, indolyl, benzofuranyl, benzothienyl, carbazolyl, quinolinyl, isoquinolinyl, purinyl and the like.

But, in any case, the heteroaryl and the aryl do not cross or include each other. Thereby, according to as defined above, if one or more full carbon aromatic ring fused with a heteroaryl is a heteroaryl, but not an aryl.

“Cycloalkyl” refers to a saturated or unsaturated, but non-aromatic, cyclic alkyl group. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In certain embodiments, the cycloalkyl group can be C₃-C₁₀ cycloalkyl, such as, for example, C₃-C₆ cycloalkyl. In a preferred embodiment, the cycloalkyl group is cyclopropane, cyclopentane or cyclohexane.

“Heterocycloalkyl” refers to a saturated or unsaturated, but non-aromatic, cyclic alkyl group in which one or more carbon atoms (and any associated hydrogen atoms) are independently replaced with the same or different heteroatom and its associated hydrogen atoms, where appropriate. Typical heteroatoms to replace the carbon atom(s) include, but are not limited to, N, P O, S, and SL Where a specific level of saturation is intended, the nomenclature “heterocycloalkanyl” or “heterocycloalkenyl” is used. Typical heterocycloalkyl groups include, but are not limited to, groups derived from epoxides, imidazolidine, morpholine, piperazine, piperidine, pyrazolidine, pyrrolidine, quinuclidine, tetrahydrofuran, tetrahydropyran and the like. Substituted heterocycloalkyl also includes ring systems substituted with one or more oxo (═O) or oxide (—O—) substituents, such as piperidinyl N-oxide, morpholinyl-N-oxide, 1-oxo-1-thiomorpholinyl and 1,1-dioxo-1-thiomorpholinyl.

But, in any case, the heterocycloalkyl and the cycloalkyl do not cross or include each other. Thereby, according to as defined above, if one or more full carbon hydrocarbon ring fused with a hererocycloalkyl to form a bi- or multi- or spiro-cyclic ring, is still defined as a hererocycloalkyl.

“Substituted” refers to a group in which one or more hydrogen atoms are each independently replaced with the same or different substituent(s). Typical substituents include, but are not limited to, X, C₃C₂₀ alkyl, C₃-C₂₀ cycloalkyl, —OR₁₃, SR₁₃, ═O, ═S, —C(O)R₁₃, —C(S)R₁₃, ═NR₁₃, —C(O)OR₁₃, —C(S)OR₁₃, —NR₁₃R₁₄, —C(O)NR₁₃R₁₄, cyano, nitro, —S(O)2R₁₃, —OS(O2)OR₁₃, —OS(O)2R₁₃, —OP(O)(OR₁₃(OR₁₄); wherein each X is independently a halogen (F, Cl, Br or I), and R₁₃ and R₁₄ is independently selected from —H, lower alkyl, lower haloalkyl. In some embodiments, the substituent(s) is independently selected from the group consisting of —F, —Cl, —Br, —I, —OH, trifluromethoxy, ethoxy, propyloxy, iso-propyloxy, n-butyloxy, isobutyloxy, t-butyloxy, —SCH₃, —SC₂H₅, formaldehyde group, —C(OCH₃), cyano, nitro, CF₃, —OCF₃, amino, dimethylamino, methylthio, sulfonyl and acetyl. Particularly preferred substituent(s) is —F, —Cl or —Br.

“Halogen” refers to fluorine (F), chlorine (Cl), bromine (Br) or iodine (I) atoms.

“Halo” refers to a fluoro, chloro, bromo, or iodo group.

“lower alkoxy” refers to a group in which the above-mentioned “lower alkyl” is bonded to an oxygen atom, and can be, a straight or branched chain alkoxy group having 1 to 6 carbon atoms such as n-butoxy isobutoxy, s-butoxy, tert-butoxy, n-pentoxy, isopentoxy, 2-methylbutoxy, neopentoxy, n-hexyloxy, 4-methylpentoxy, 3-methylpentoxy, 2-methylpentoxy, 3,3-dimethylbutoxy, 2,2-dimethylbutoxy, 1,1-dimethylbutoxy, 1,2-dimethylbutoxy, 1,3-dimethylbutoxy, or 2,3-dimethylbutoxy. The lower alkoxy group is preferably a methoxy or ethoxy group.

“alkylsulfonylalkoxy” refers to a group in which the above-mentioned “alkyl” is bonded to a sulfonyl group and the sulfonyl group bonded to the above-mentioned “alkoxy” group. For example, methylsulfonylmethoxyl, methylsulfonyloxethyl, methylsulfonylpropoxy, methylsulfonylbutoxy, ethylsulfonylmethoxyl, ethylsulfonyloxethyl, ethylsulfonylpropoxy, ethylsulfonylbutoxy, propylsulfonylmethoxyl, propylsulfonyloxethyl, propylsulfonylpropoxy, propylsulfonylbutoxy, butylsulfonylmethoxyl, butylsulfonyloxethyl, butylsulfonylpropoxy or butylsulfonylbutoxy. The alkylsulfonylalkoxy group is preferably a methylsulfonylpropoxy or methylsulfonylbutoxy, and most preferably a methylsulfonylpropoxy.

“C₆-C₁₄ aryloxy group” refers to a group in which the above-mentioned “C₆-C₁₄ aryl group” is bonded to an oxygen atom, and can be, for example, phenoxy, 1-naphthoxy, 2-naphthoxy. The C₆-C₁₄ aryloxy group is preferably a phenoxy.

“C₇-C₁₆ is aralkyl” refers to a group in which the above-mentioned “C₇-C₁₆ aryl group” is bonded to the above-mentioned “Alkyl group”. Typical aralkyl groups include, but are not limited to, groups derived from benzyl, phenemyl, hydrocinnamyl, benzhydryl, 1-methylnaphthalene or 2-methylnaphthalene. The C₇-C₁₆ aralkyl group is preferably a benzyl.

“aralkyloxy” refers to a group in which the above-mentioned “aralkyl” group is bonded to an oxygen atom, and can be, for example, benzyloxy or Phenylethoxy.

“alkylthioalkoxy” refers to a group in which the above-mentioned “alkyl” is bonded to a thio group and the thio group bonded to the above-mentioned “alkoxy” group. For example, methylthiomethoxyl, methylthiooxethyl, methylthiopropoxy, methylthiobutoxy, ethylthiomethoxyl, ethylthiooxethyl, ethylthiopropoxy, ethylthiobutoxy, propylthiomethoxyl, propylthiooxethyl, propylthiopropoxy, propylthiobutoxy, butylthiomethoxyl, butylthiooxethyl, butylthiopropoxy or butylthiobutoxy. The alkylthioalkoxy group is preferably a methylthiopropoxy or methylthiobutoxy, and most preferably a methylthiopropoxy.

“alkoxyalkoxy” refers to a group in which the above-mentioned “alkoxy” is bonded to another “alkoxy” group. Typical aralkyl groups include, but are not limited to, groups derived from methoxylmethoxyl, methoxyloxethyl, methoxylpropoxy, methoxylbutoxy, oxethylmethoxyl, oxethyloxethyl, oxethylpropoxy or oxethylbutoxy. The alkoxyalkoxy group is preferably a methoxylpropoxy or methoxylbutoxy, and most preferably a methoxylpropoxy.

“hydroxyalkyl” refers to one or more hydrogen atoms in which the above-mentioned “alkyl” are each independently replaced with the hydroxyl group. Typical hydroxyalkyl groups include, but are not limited to, groups derived from hydroxymethyl, hydroxyethyl, 1-hydroxyethyl, hydroxypropyl, 1-hydroxypropyl, 2-hydroxypropyl or hydroxyisopropyl.

In some embodiments of the present invention, n is selected from any integer from 0, 2, 3, 4 or 5, and most preferably n is 0 or 2. m is selected from any integer from 0, 1, 2 or 3, and most preferably m is 0. k is selected from any integer from 0, 1, 2, 3, 4 or 5, and most preferably k is 3.

The prodrug of the compound (I) is a compound which is converted to the compound (I) with a reaction due to an enzyme, gastric acid, etc. under the physiological condition in the living body, such as the corresponding ester or amide. That is, a compound which is converted to the compound (I) by enzymatic oxidation, reduction, hydrolysis, etc.; a compound which is converted to the compound (I) by hydrolysis etc. due to gastric acid, and the like.

Example of a prodrug of compound (I) include a compound wherein an amino group of compound (I) is acylated, alkylated, phosphorylated or borated. A compound wherein a carboxyl group of compound (I) is esterified or amidated and the like. Of these, a compound wherein a carboxyl group of compound (I) is esterified by C₁-C₆ alkyl group such as methyl, ethyl, tert-butyl and the like is preferable. These compounds can be produced from compound (I) according to a method known per se.

As used herein, “pharmaceutically acceptable” refers to generally recognized for use in animals, and more particularly in humans.

As a salt of compound (I), for example, metal salts, an ammonium salt, salts with organic bases, salts with inorganic acids, salts with organic acids, salts with basic or acidic amino acids and the like can be mentioned. Preferable examples of the metal salt include alkali metal salts such as sodium salt, potassium salt and the like; alkaline earth metal salts such as calcium salt, magnesium salt, barium salt and the like; aluminum salt and the like.

Preferable examples of the salt with organic base include a salt with trimethylamine, triethylamine, pyridine, picoline, 2,6-lutidine, ethanolamine, diethanolamine, triethanolamine, cyclohexylamine, dicyclohexylamine, N,N-dibenzylethylenediamine, choline, 2-(diethylamino)ethanol, N-methylglucamine, tromethamine, 1H-imidazole; Piperazine, N-hydroxyethyl morpholin, 1-(2-Hydroxyethyl)pyrrolidine, Tris(Hydroxymethyl)aminomethane and the like.

Preferable examples of the salt with inorganic acid include a salt with hydrochloric acid, hydrobromic acid, nitric acid, sulfuric acid, phosphoric acid and the like.

Preferable examples of the salt with organic acid include a salt with formic acid, acetic acid, trifluoroacetic acid, phthalic acid, fumaric acid, oxalic acid, tartaric acid, maleic acid, citric acid, succinic acid, malic acid, methanesulfonio acid, benzenesulonic acid, p-toluenesulfonic acid and the like.

Preferable examples of the salt with basic amino acid include a salt with arginine, lysine, ornithine, histidine and the like. Preferable examples of the salt with acidic amino acid include a salt with aspartic acid, glutamic acid and the like.

Of the above-mentioned salts, a pharmacologically acceptable salt is preferable.

“Therapeutically effective amount” refers to the amount of a compound that, when administered to a subject for treating a disease, or at least one of the clinical symptoms of a disease or disorder, is sufficient to affect such treatment for the disease, disorder, or symptom. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

The pharmaceutically acceptable carrier of the present invention refers to a conventional pharmaceutical carrier in the pharmaceutical art, for example: diluents, excipients such as water, fillers such as starch, sucrose, etc.; binders such as cellulose derivatives, alginic acid, gelatin, and polyvinyl pyrrolidone; wetting agents such as glycerol; disintegrating agents such as agar-agar, calcium carbonate, and sodium bicarbonate; absorption accelerators such as quaternary ammonium compounds; surface active agents such as cetyl alcohol; adsorptive carriers such as kaolin and bentonite clay; lubricants such as talc, calcium and magnesium stearate, and polyethylene glycol. It also can add other adjuvants such as flavoring agents, sweeteners, etc. in the composition.

Examples of the present compound (I) in a crystal form can be present in both forms of nonsolvate and solvate. By using different solvents crystallization may get different pharmaceutical acceptable solvates. Examples of the solvate include inorganic solvates and organic solvates with water, methanol, ethanol, isopropyl alcohol, acetone, acetonitrile, ethylacetate or the like. From the aspects of pharmaceutical ingredient, hydrate is more preferable than solvate with an organic solvent. In addition, according to the number of the solvents for the present compound, solvates of hemi-, mono-, di-, tri-, tefra-, penta-, hexa- and the like can be present. In the case of a hydrate, preferred is a hydrate of not more than 3, more preferably 1 or 2 hydrate.

It will be appreciated by the skilled artisan that some of the compounds of formula (I) may contain one or more chiral centers and therefore exist in two or more stereoisomeric forms. The racemates of these isomers, the individual isomers and mixtures enriched in one enantiomer, as well as diastereomers when there are two chiral centers, and mixtures partially enriched with specific diastereomers are within the scope of the present invention. The present invention includes all the individual stereoisomers (e.g. enantiomers), racemic mixtures or partially resolved mixtures of the compounds of formulae (I) and, where appropriate, the individual tautomeric forms thereof.

The compound of the present invention can be apphed to patients in need of such treatment in the form of composition by oral, nasal inhalation, rectal or parenteral administration. For oral administration, it can be made into conventional solid preparations such as tablets, powders, granules, capsules and the like, or made into liquid preparations, such as water or oil suspensions or other liquid preparations such as syrups, elixirs and the like; for parenteral administration, it can be made into injection solutions, aqueous or oily suspensions and the like. Preferably, the composition is in the form of tablets, capsules and injections.

The various dosage forms of the pharmaceutical composition of the present invention can be prepared in accordance with conventional production method in pharmaceutical field. For example, the active ingredient is mixed with one or more carriers, and then formed into the desired dosage form.

### EXAMPLES

The present invention is further exemplified, but not limited, by the following examples that illustrate the preparation of compounds of the invention.

The following examples are only used to describe some embodiments of the current invention to make the current invention more fully appreciated and understood by those skilled in the art, but should not limit the spirit and scope of the current invention in any way. The scope of the invention is defined by the appended claims. In the detalled description of the preferred embodiments of the current invention, those technologies or methods not stated expressly are those that are regular and apparent to those skilled in the art.

### Example 1

### 2-((S)-6-(((S)-4-(4-(3-methoxypropoxy)-2,6-dimethylphenyl)-2,3-dihydro-1H-inden-1-yl)oxy)-2,3-dihydrobenzo[b]thiophen-3-yl)acetic acid

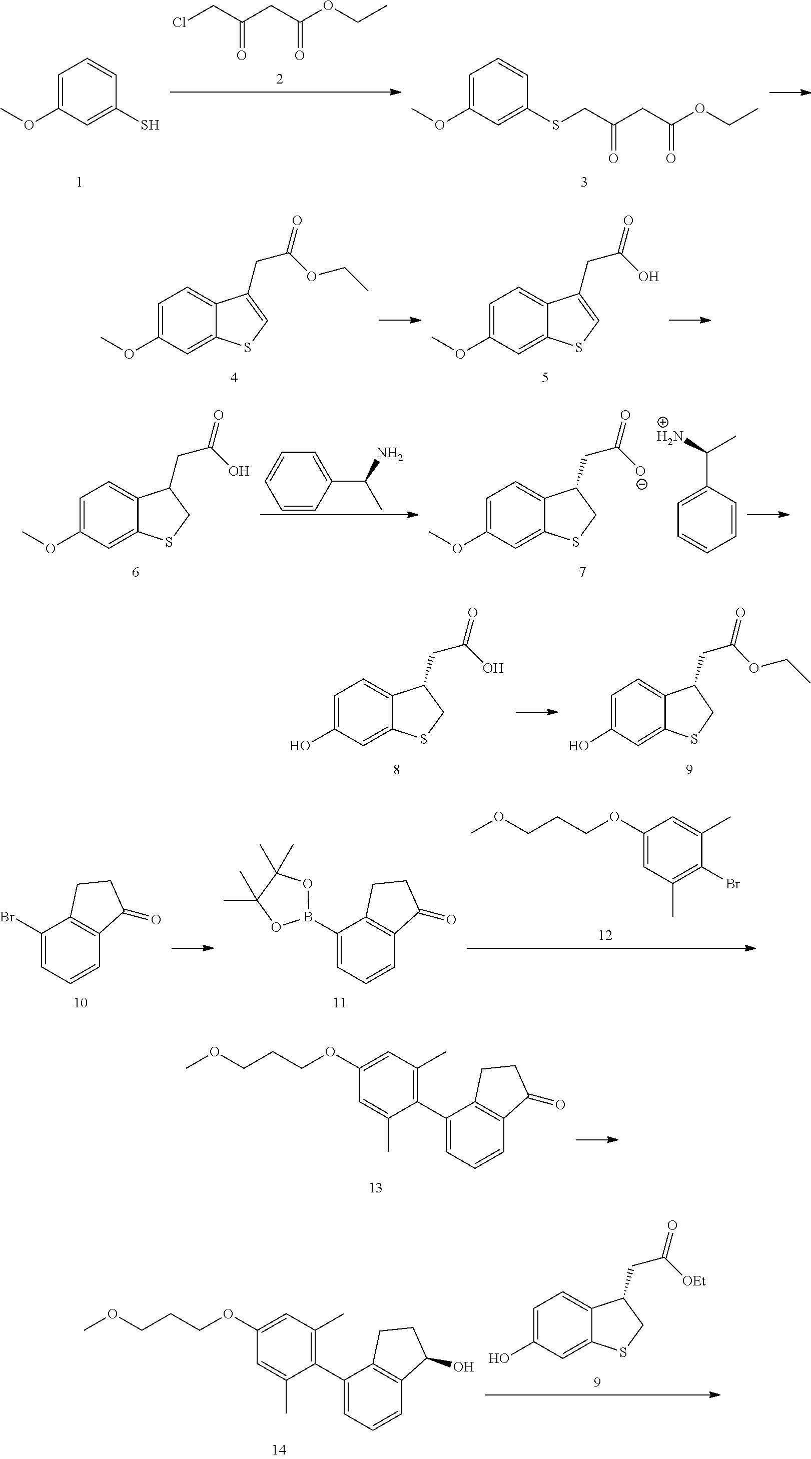

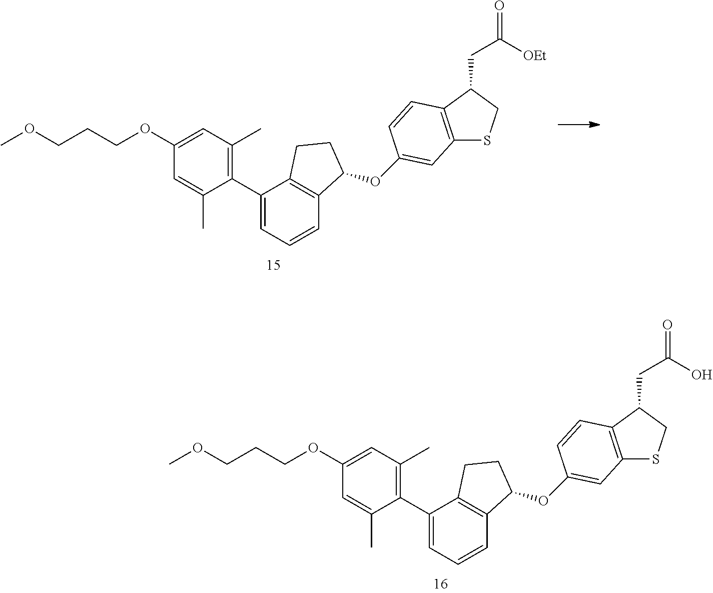 [SMILES: CCOC(=O)C[C@@H]1CSc2cc(O[C@H]3CCc4c(-c5c(C)cc(OCCCOC)cc5C)cccc43)ccc21 ; COCCCOc1cc(C)c(-c2cccc3c2CC[C@@H]3Oc2ccc3c(c2)SC[C@H]3CC(=O)O)c(C)c1]

Step A: 3-Methoxybenzenethiol (28 g, 0.2 mol), potassium carbonate (41.4 g, 0.3 mol) and 180 ml of anhydrous ethanoi were added into a 500 mL flask. The reaction mixture was cooled to 0° C. in ice-bath, stirred for 15 min, and then was added Ethyl 4-chloroacetoacetate (36.3 g, 0.22 mol) in drops in ice-bath, and stirred at room temperature for 2 hr. After filtered, the filtrate went through vacuum distillation to give a yellow oil of product 3.

Step B: The product 3 obtained in step A was added into methanesulfonic acid (35 mL) in drops in ice-bath, and stirred at room temperature overnight. The reaction mixture was poured into 250 mL ice water and was extracted by 3×120 mL ethyl acetate. The organic phase was washed by 180 mL saturated Na₂CO₃ solution, dried by anhydrous Na₂SO₄ and distilled under vacuum to obtain a brown oil of product 4.

Step C: The product 4 obtained in step B was added NaOH solution (17.4 g in 200 mL wafer) in ice-bath, and stirred at room temperature for 2 hr, then concentrated hydrochloric acid solution was added with pH value adjusted to 3 and filtered. The resulted solid was added 200 mL ethyl acetate and 200 mL water, and stirred for 0.5 hr. After separation, the organic phase was distilled under vacuum to obtain a crude product. 52 mL ethyl acetate was added and slurried for 0.5 hr, filtered, the filter cake washed with ethyl acetate and petroleum ether (PE/EA=3/1) to give product 5 (17 g),

Step D: The product 5 obtained in step C was added 68 mL trifluoroacetic acid and 19 mL triethylsilane, and stirred at 50° C. overnight. After the solvent removed, the mixed solution of petroleum ether and ethyl acetate (PE/EA=10 mL/10 mL) was added, stirred for 0.5 hr, and filtered to obtain a crude product 6 (11.2 g, yield 85%).

Step E: The product 8 obtained in step D was added 15 mL methanol and 6.05 g (S)-(-)-1-Phenylethylamine. The mixture was stirred at room temperature for 0.5 hr and heated to reflux for 1 hr. Methanol was added till all of the solid was dissolved. Then cooled to room temperature and stirred overnight, filtered. The precipitated crystals were recrystallized three times to obtain white crystals of product 7 (2.6 g, 98% ee).

Step F: The product 7 obtained in step E was added 7 mL 48% hydrobromic acid and heated to reflux overnight, then cooled to room temperature. 10 mL water was added and the mixture was extracted by 3×15 mL ethyl acetate. The organic phase were combined and washed by saturated NaCl solution (20 mL×2), dried by anhydrous Na₂SO₄ and distilled under vacuum to obtain a purple solid of product 8.

Step G: The product 8 obtained in step F was added 10 mL anhydrous ethanol and one drop of concentrated sulfuric acid. The mixture was heated to reflux for 2 hr, then cooled to room temperature. 20 mL water was added and the mixture was extracted by 3×20 mL methylene chloride. The organic phase was washed by saturated NaHCO₃ solution (20 mL) and saturated NaCl solution (20 mL), dried by anhydrous Na₂SO₄ and distilled under vacuum to obtain a purple oil product 3 (1.27 g).

Step H: Under nitrogen gas, 4-bromo-1-indanone (8 g, 37.9 mmol), bis(pinacolato)diboron (11.55 g; 45.4 mmol), potassium acetate (11.18 g, 113.7 mmol), 1,1′-bis(diphenylphosphino)ferrocene-palladium(II)dichloride dichloromethane complex (1.548 g, 1.89 mmol), and 180 mL 1,4-dioxane were added into a 500 mL 3-neck flask. The mixture was heated to reflux at 80° C. (and stirred for 7 hr, then cooled to room temperature, and concentrated by rotary evaporation. 120 mL water was added and the mixture was extracted by 100 ml×3 ethyl acetate. The combined organic phase was washed by saturated NaCl solution, dried by anhydrous Na₂S₄ and evaporated under vacuum to remove solvent, to give a crude product. The product was purified by column chromatography (ethyl acetate: petroleum ether=10:90) to obtain a white solid product 11 (8.8 g, yield 90%).

Step I: Under nitrogen gas, product 11 (2.786 g, 10.8 mmol), 2-bromo-5-(3-methoxypropoxy)-1,3-dimethylbenzene (2.457 g, 9 mmol), potassium carbonate (3.726 g, 27 mmol), tetrakis(triphenylphosphine)palladium (0.52 g, 0.45 mmol), 40 mL 1,4-dioxane and 8 mL water were added into a 250 mL 2-neck flask. The mixture was heated to reflux at 90° C. and stirred for 20 hr, then cooled to room temperature, concentrated by rotary evaporation. 50 mL water was added and the mixture was extracted by 50 ml×3 ethyl acetate. The combined organic phase was washed by saturated NaCl solution, dried by anhydrous Na₂SO₄ and evaporated under vacuum to remove solvent, to give a crude product. The product was purified by column chromatography (ethyl acetate: petroleum ether=10:90) to obtain a light yellow solid product 13 (2.18 g, yield 75%).

Step J: Under nitrogen gas, (S)-2-Methyl-CBS-Oxazaborolidine (1.2 mL, 1.2 mmol), BH₃-Me₂S (0.9 mL, 9 mmol) and 15 ml methylene chloride were added into a 100 mL 3-neck flask, then cooled to −25° C. A solution of product 13 (1.94 g, 8 mmol) in 10 mL methylene chloride was added dropwise over 30 min. The reaction was stirred for 4 hr after the addition was complete, then quenched by the dropwise addition of MeOH (10 mL). The mixture was warmed to room temperature and evaporated under vacuum to remove solvent. The product was purified by column chromatography (ethyl acetate:hexane=25:75) to obtain a oil product 14 (1.76 g, yield 90%).

Step K: Under nitrogen gas, product 14 (1.0 g, 3.1 mmol), product 9 (0.73 g, 3.1 mmol), triphenylphosphine (1.6 g, 6.2 mmol) and 10 mL toluene were added into a 100 mL 2-neck flask. The mixture was cooled to 0° C. and stirred for 30 min, added DEAD (1.1 g, 6.2 mmol) in drops at 0° C. and stirred at room temperature for 15 hr, concentrated by rotary evaporation, 20 mL water was added and was extracted by 20 mL×3 ethyl acetate. The combined organic phase was washed by saturated NaCl solution, dried by anhydrous Na₂SO₄ and evaporated under vacuum to remove solvent, to give a crude product. The product was purified by column chromatography (ethyl acetate:hexane=10:90) to obtain a white solid product 15 (0.84 g, yield 50%).

Step L: Product 15 (0.84 g, 1.5 mmol) was added into the mixed solution of THF and MeOH (5 mL/5 mL), then added 3 mL NaOH (240 mg; 6.0 mmol) aqueous solution, stirred at room temperature for 15 hr. The reaction mixture was acidified with 1N HCl aqueous solution with pH value adjusted to 3, then evaporated under vacuum to remove solvent and was further extracted by 20 mL×3 ethyl acetate. The combined organic phase was washed by saturated NaCl solution, dried by anhydrous Na₂SO₄ and evaporated under vacuum to remove solvent, to give a white solid product 16 (0.75 g, yield 95%). LC-MS[M+H]-m/z is 519.

### Example 2

### (S)-2-(-6-((2′,6′-dimethyl-4′-(3(methylsulfonyl)porpoxy)-[1,1′-biphenyl]-3-yl)methoxy)-2,3-dihydrofuro[3,2-c]pyridin-3-yl)acetic acid

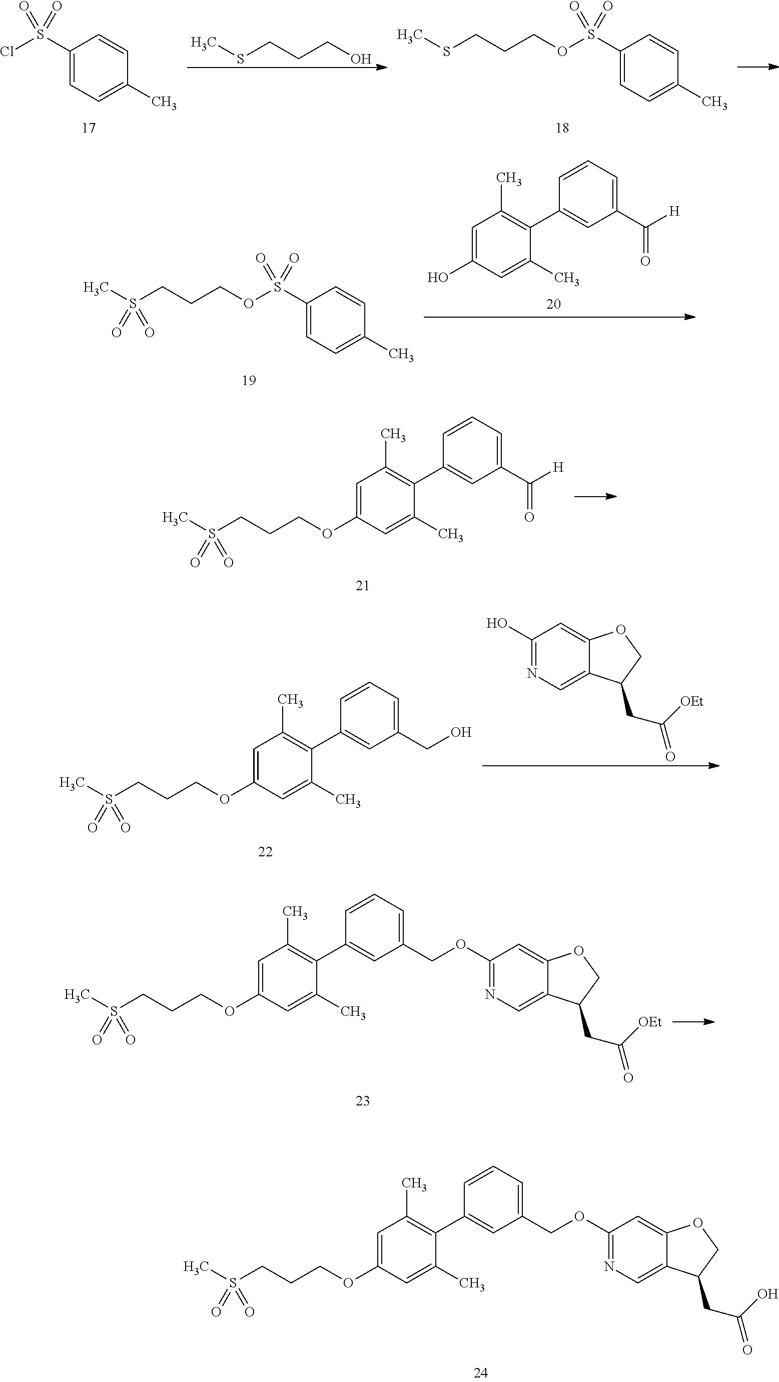

Step A: A solution of 3-(methylthio)-1-propanol (3.18 g, 30.0 mmol), triethylamine (6.30 mL, 45.0 mmol) and N,N,N,N′-tetramethyl-1,6-hexanediamine (0.520 g, 3.00 mmol) in toluene (50 mL) was ice-cooled, and a solution of p-toluenesulfonyl chloride in toluene was added dropwise under nitrogen atmosphere. After completion of the dropwise addition, the mixture was stirred for 3 hr, during which the mixture was allowed to warm to room temperature. Water was added to the reaction mixture, and the mixture was extracted with ethyl acetate. The extract was washed with saturated brine, dried over anhydrous sodium sulfate, and concentrated under reduced pressure. The residue was purified by column chromatography (ethyl acetate:hexane=10:90-40:80) to obtain a colorless oil product 18.

Step B: To a solution of product 18 (7.32 g, 28.1 mmol) in methanol (150 mL) was added dropwise a solution of potassium peroxysulfate (34.6 g, 56.3 mmol) in water (150 mL) under ice-cooling. After completion of the dropwise addition, the mixture was stirred for 20 hr. during which the mixture was allowed to gradually warm to room temperature. Methanol was evaporated under reduced pressure, and the mixture was diluted with water, and the organic material was extracted with ethyl acetate. The extract was washed with saturated brine, dried over anhydrous sodium sulfate, and concentrated under reduced pressure. The precipitated crystals were washed with ethyl acetate-heptane to give a colorless crystal product 19 (7.86 g, yield 95%).

Step C: 4-Bromo-3,5-dimethylphenol (6.18 g, 30.6 mmol) and (3-formylphenyl)boronic acid (4.60 g, 30.7 mmol) were dissolved in a mixture of 1 M aqueous sodium carbonate solution (90 mL), ethanol (30 mL) and toluene (90 mL). After argon substitution, tetrakis(triphenylphosphine)palladium (1.77 g, 1.53 mmol) was added , and the reaction mixture was stirred at 80° C. for 24 hr. The reaction mixture was allowed to cool, and water was added. The mixture was diluted with ethyl acetate, and the insoluble substance was filtered off through celite. The organic layer of the filtrate was washed with saturated brine, dried over anhydrous sodium sulfate, and concentrated under reduced pressure. The residue was purified by column chromatography (ethyl acetate:hexane=10:90-40:60) to obtain a pale-yellow product 20 (5.71 g, yield 92%).

Step D: To a solution of product 20 (1.36 g, 6.00 mmol) and product 19 (2.11 g, 7.20 mmol) in N,N-dimethylformamide (12 mL) was added potassium carbonate (1.08 g, 7.80 mmol), and the mixture was stirred at 90° C. for 24 hr under nitrogen atmosphere. Water was added to the reaction mixture, and the mixture was extracted with ethyl acetate. The extract was washed successively with 1 M aqueous sodium hydroxide solution and saturated brine, dried over anhydrous magnesium sulfate, and concentrated under reduced pressure. The residue was purified by column chromatography (ethyl acetate:hexane=40:80-80:20), and the obtained crystals were recrystallized from heptane-ethyl acetate to give a colorless solid product 21 (1.61 g, yield 78%).

Step E: A solution of product 21 (1.60 g, 4.61 mmol) in a mixed solvent of methanol (10 mL) and tetrahydrofuran (20 mL) was ice-cooled, sodium borohydride (90%, 0.194 g, 4.61 mmol) was added, and the mixture was stirred for 6 hr under nitrogen atmosphere. The reaction mixture was treated with diluted hydrochloric acid, and extracted with ethyl acetate The extract was washed with saturated brine, dried over anhydrous magnesium sulfate, and concentrated under reduced pressure.The obtained crystals were recrystallized from heptane-ethyl acetate to give a colorless solid product 22 (1.56 g, yield 97%).

Step F: A solution of ethyl (S)-2-(6-hydroxy-2,3-dihydrofuro[3,2-c]pyridin-3-yl)acetate (0.267 g, 1.20 mmol), product 22 (0.418 g, 1.20mmol) and tributylphosphine (0.389 g, 1.92 mmol) in toluene (15 mL) was stirred, 1,1′-(azodicarbonyl)dipiperidine (0.485 g, 1.92 mmol) was added, and the mixture was stirred at room temperature for 1.5 hr under nitrogen atmosphere. Hexane (8 mL) was added to the reaction mixture, the precipitated insoluble substante was filtered off, and the filtrate was concentrated under reduced pressure. The residue was purified by column chromatography (ethyl acetate:hexane=40:60-80:20) to give a colorless solid product 23 (0.530 g, yield 82%).

Step G: To a solution of product 23 (0.539 g, 0.976 mmol) in a mixed solvent of methanol (2 mL) and tetrahydrofuran (4 mL) was added 2M aqueous sodium hydroxide solution (1 mL), and the mixture was stirred at 50° C. for 2 hr. The reaction mixture was diluted with water, acidified with 1 M hydrochloric acid, and extracted with ethyl acetate. The extract was washed with saturated brine, dried over anhydrous magnesium sulfate, and concentrated under reduced pressure. The precipitated crystals were recrystallized from heptane-ethyl acetate to give a colorless solid product 24 (0.452 g, yield 88%) as colorless crystals. LC-MS[M+H]-m/z is 526.

### Example 3

### 2-((3S)-6-((4-(2,6-dimethyl-4-(3-(methylsulfonyl)propoxy)phenyl)-2,3-dihydro-1H-inden-1-yl)oxy)-2,3-dihydrofuro[3,2-c]pyridin-3-yl)acetic acid

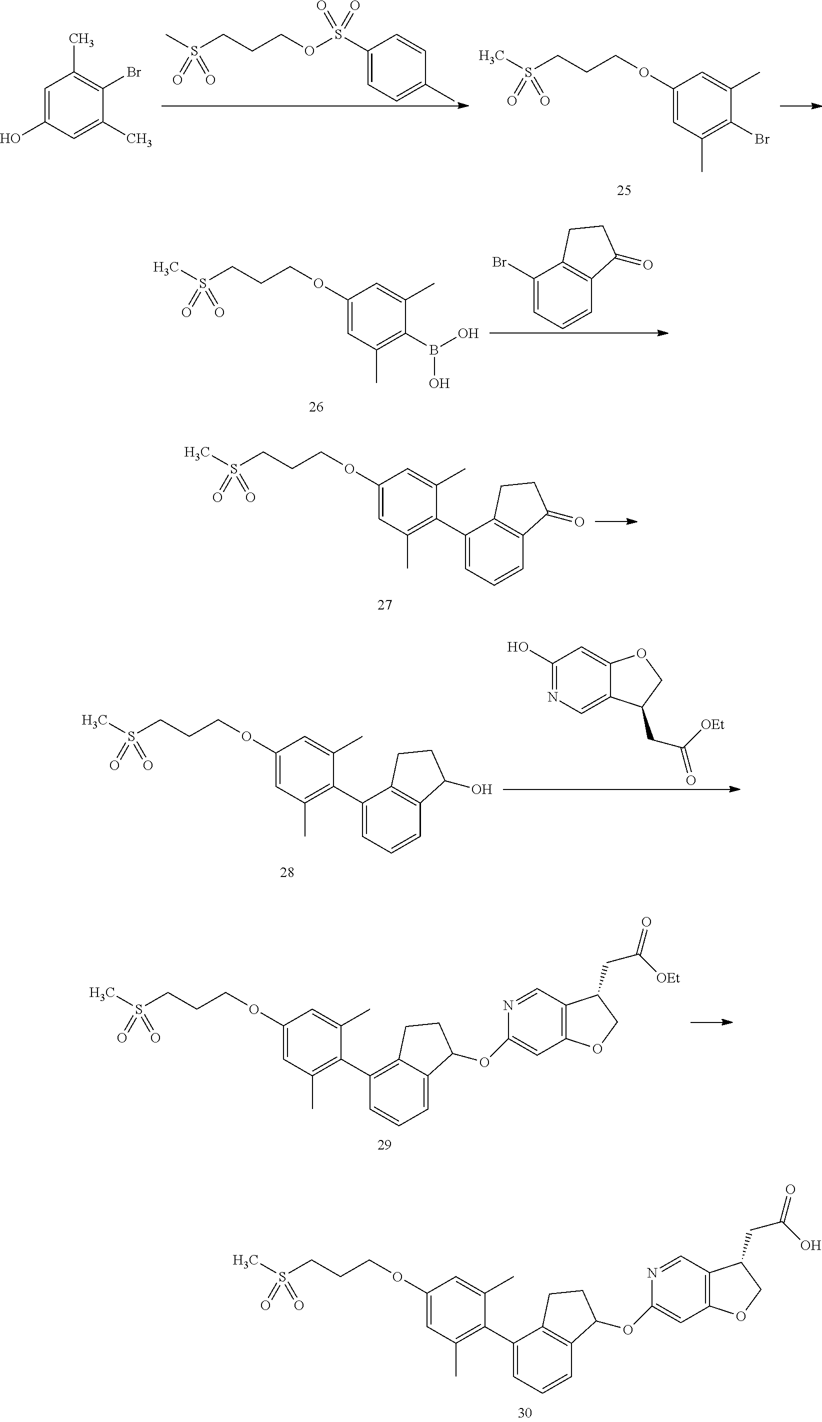

Step A: To a solution of 4-bromo-3,5-dimethylphenol (2.01 g, 10 mmol) and 3-(methylsulfonyl)propyl 4-methylbenzenesulfonafe (3.51 g, 12.0 mmol) in N,N-dimethylformamide (20 mL) was added potassium carbonate (1.80 g, 13.0 mmol), and the mixture was stirred at 90° C. for 24 hr under nitrogen atmosphere. Water was added to the reaction mixture, and the mixture was extracted with ethyl acetate. The extract was washed successively with 1M aqueous sodium hydroxide solution and saturated brine, dried over anhydrous magnesium sulfate, and concentrated under reduced pressure. The residue was purified by column chromatography (ethyl acetate:hexane=40:60-80:20), and the obtained crystals were recrystallized from heptane-ethyl acetate to give a colorless crystals product 25 (2.73 g/yield 85%).

Step B: To a solution of product 25 (3.21 g, 10.0 mmol) in tetrahydrofuran (50 mL) was added dropwise n-butyllithium hexane solution (1.6M, 6.57 mL, 10.5 mmol) at −78° C., and the reaction mixture was stirred for 1.5 hr at the same temperature. Triisopropyl borate (6.92 mL, 30 mmol) was added, and the mixture was stirred overnight, during which the mixture was allowed to room temperature. The reartion mixture was ice-cooled, 2 M hydrochloric acid (50 mL) was added, and the mixture was stirred for 2.5 hr. The aqueous layer and the organic layer were separated, and the organic layer was washed with saturated brine and saturated aqueous sodium hydrogencarbonate while simultaneously adjusting to neutral. The organic layer was dried over anhydrous magnesium sulfate, and concentrated under reduced pressure. The residue was washed with cool hexane to give the colorless crystals product 26 (1.91 g, 67%).

Step C: Product 26 (8.64 g, 30.2 mmol) and 4-bromo-2,3-dihydro-1H-inden-1-one (6.33 g, 30 mmol) were dissolved in a mixture of 1M aqueous sodium carbonate solution (90 mL), ethanol (30 mL) and toluene (90 mL). After argon substitution, tetrakis(triphenylphosphine)palladium(0) (1.74 g, 1.5 mmol) was added, and the reaction mixture was stirred at 80° C. for 24 hr under argon atmosphere. The reaction mixture was allowed to cool, and water was added. The mixture was diluted with ethyl acetate, and the insoluble substante was fittered off through celite. The organic layer of the filtrate was washed with saturated brine, dried over anhydrous magnesium sulfate, and concentrated under reduced pressure. The residue was purifiedby silica gel column chromatography (ethyl acetate:hexane=10:90-40:60) to give the pale-yellow compound 27 (9.72 g, yield 83%).

Step D: To a solution of product 27 (3.72 g, 10 mmol) in a mixed solvent of methanol (8 mL) and tetrahydrofuran (12 mL) was added sodium borohydride (0.378 g, 10 mmol), and the mixture was stirred overnight at room temperature. The reaction mixture was concentrated under reduced pressure, added water, and extracted with ethyl acetate. The organic layer dried by anhydrous Na₂SO₄ and concentrated under reduced pressure. The residue was purified by silica gel column chromatography (ethyl acetate:hexane=5:95-40:60) to give the pale-yellow oil product 28 (3.17 g, yield 85%).

Step E: A solution of ethyl (S)-2-(6-hydroxy-2,3-dihydrofuro[3,2-c]pyridin-3-yl)acetate (0.747 gs 3.35 mmol), product 28 (1.87 g, 5 mmol) and triphenylphosphine (1.30 g, 5 mmol) in tetrahydrofuran (20 mL) was stirred, 40% diethyl azodicarboxylate (2.25 mL, 5 mmol) in toluene was added, and the mixture was stirred at 50° C. for 3 hr under nitrogen atmosphere. The reaction mixture was concentrated under reduced pressure. The residue was purified by column chromatography (ethyl acetate:hexane=5:95-20:80) to give a product 29 (1.59 g, yield 82%).

Step F: To a solution of product 29 (1.70 g, 5 mmol) in a mixed solvent of methanol (5 mL) and tetrahydrofuran (5 mL) was added 2M aqueous sodium hydroxide solution (4.5 mL, 9 mmol), and the mixture was stirred at 50° C. for 1.5 hr. The reaction mixture was diluted with water, acidified with 1 M hydrochloric acid, and extracted with ethyl acetate. The extract was washed with saturated brine, dried over anhydrous magnesium sulfate, and concentrated under reduced pressure. The precipitated crystals were recrystallized from heptane-ethyl acetate to give a product 30 (1.49 g, yield 90%). LC-MS[M+H]-m/z is 552.

### Example 4

### (S)-2-(6-((2′,6′-dimethyl-4′-(3-(methylhio)propoxy)-[1,1′-biphenyl]-3-yl)methoxy)-2,3-dihydrofuro[3.2-c]pyridin-3-yl)acetic acid

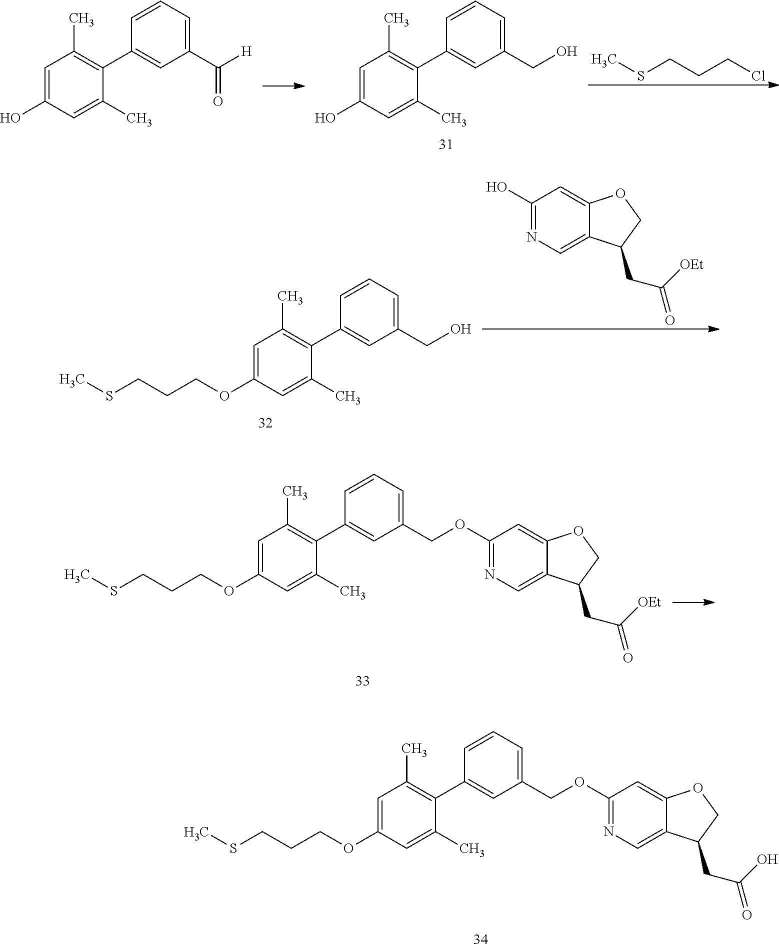

Step A: A solution of 4′-hydroxy-2′,6′-dimethyl-[1,1′-biphenyl]-3-carbaldehyde (5.56 g, 24.6 mmol) in a mixed solvent of methanol (25 mL) and tetrahydrofuran (50 mL) was ice-cooled, sodium borohydride (90%, 1.03 g, 24.6 mmol) was added, and the mixture was stirred for 20 hr under nitrogen atmosphere. The reaction mixture was concentrated under reduced pressure and treated with diluted hydrochloric acid, and extracted with ethyl acetate. The extract was washed with saturated brine, dried over anhydrous magnesium sulfate, and concentrated under reduced pressure. The obtained crystals were recrystallized from heptane-ethyl acetate to give a colorless solid product 31 (5.25 g, yield 93%).

Step B: To a solution of product 31 (1.46 g; 6.40 mmol) and 3-chloropropyl methyl sulfane (0.856 mL, 9.60 mmol) in N,N-dimethylformamide (15 mL) were added potassium carbonate and potassium iodide, and the mixture was stirred at 95° C. for 24 hr under nitrogen atmosphere. Water was added to the reaction mixture, and the mixture was extracted with ethyl acetate. The extract was washed successively with 1 M aqueous sodium hydroxide solution and saturated brine, dried over anhydrous magnesium sulfate, and concerttrated under reduced pressure. The residue was purified by silica gel column chromatography (ethyl acetate:hexane=10:90-50:50) to give the title compound as a colorless oil product 32 (0.95 g, yield 47%).

Step C: A solution of ethyl (S)-2-(6-hydroxy-2,3-dihydrofuro[3,2-c]pyridin-3-yl)acetate (0.535 g, 2.40 mmol), product 32 (0.760 g, 2.40 mmol) and tributylphosphine (0.776 g, 3.84 mmol) in toluene (40 mL) was stirred, 1,1-(azodicarbonyl)dipiperidine (0.968g, 3.84 mmol) was added, and the mixture was stirred at room temperature for 1 hr under nitrogen atmosphere. Hexane (20 mL) was added to the reaction mixture, the precipitated insoluble substante was filtered off, and the filtrate was concentrated under reduced pressure. The residue was purified by column chromatography (ethyl acetate:hexane=5:95-40:60) to give a pale-yellow oil product 33 (0.730 g, yield 60%).

Step D: To a solution of product 33 (0.507 g, 1 mmol) in a mixed solvent of methanol (3 mL) and tetrahydrofuran (6 mL) was added 2M aqueous sodium hydroxide solution (1.10 mL), and the mixture was stirred at 50° C. for 1.5 hr. The reaction mixture was diluted with water, acidified with 1 M hydrochloric acid, and extracted with ethyl acetate. The extract was washed with saturated brine, dried over anhydrous magnesium sulfate, and concentrated under reduced pressure to give a colorless oil product 34 (0.428 g, yield 87%). LC-MS[M+H]-m/z is 494.

### Example 5

### 2-((3S)-6-((4-(2,6-dimethyl-4-(3-(methylthio)propoxy)phenyl)-2,3-dihydro-1H-inden-1-yl)oxy)-2,3-dihydrofuro[3,2-c]pyridin-3-yl)acetic acid

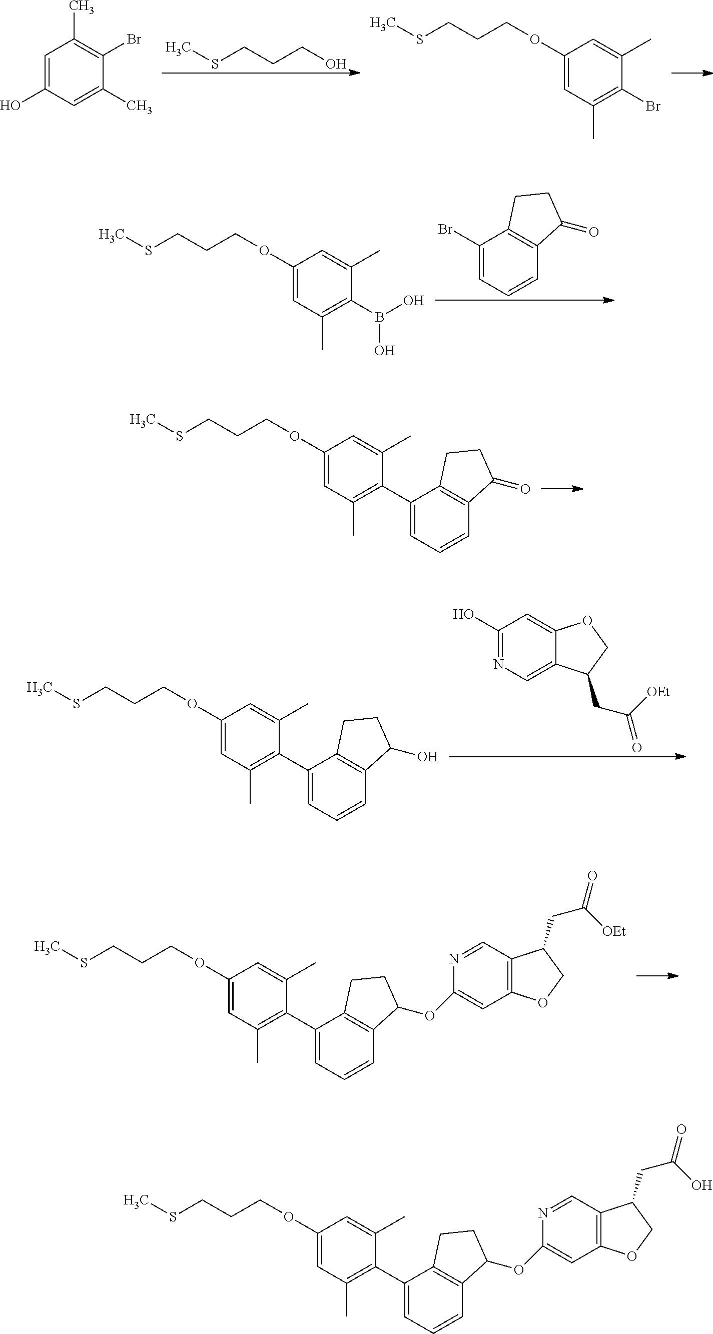

A solution of 4-bromo-3,5-dimethylphenol (5.03 g; 25 mmol), 3-(methylthio)propan-1-ol (2.65 g; 25 mmol) and tributylphosphine (9.96 mL; 40 mmol) in toluene (320 mL) was stirred, 1,1′-(azodicarbonyl)dipiperidine (10.1 g, 40 mmol) was added, and the mixture was stirred at room temperature for 18 hr under nitrogen atmosphere. Hexane (160 mL) was added to the reaction mixture, the precipitated insoluble substante was filtered off, and the filtrate was concentrated under reduced pressure. The residue was purified by column chromatography (ethyl acetate:hexane=0:100-25:75) to give a pale-yellow oil of (3-(4-bromo-3,5-dimethylphenoxy)propyl)(methyl)sulfane (6.29 g, yield 87%). The subsequent synthetic procedure are similar to Example 3 was followed to arrive at the title compound, with the LC-MS[M+H]-m/z as 520.

### Example 6

### 2-((3S)-6-((4-(4-(3-methoxypropoxy)-2,6-dimethylphenyl)-2,3-dihydro-1H-inden-1-yl)oxy)-2,3-dihydrofuro[3,2-c]pyridin-3-yl)acetic acid

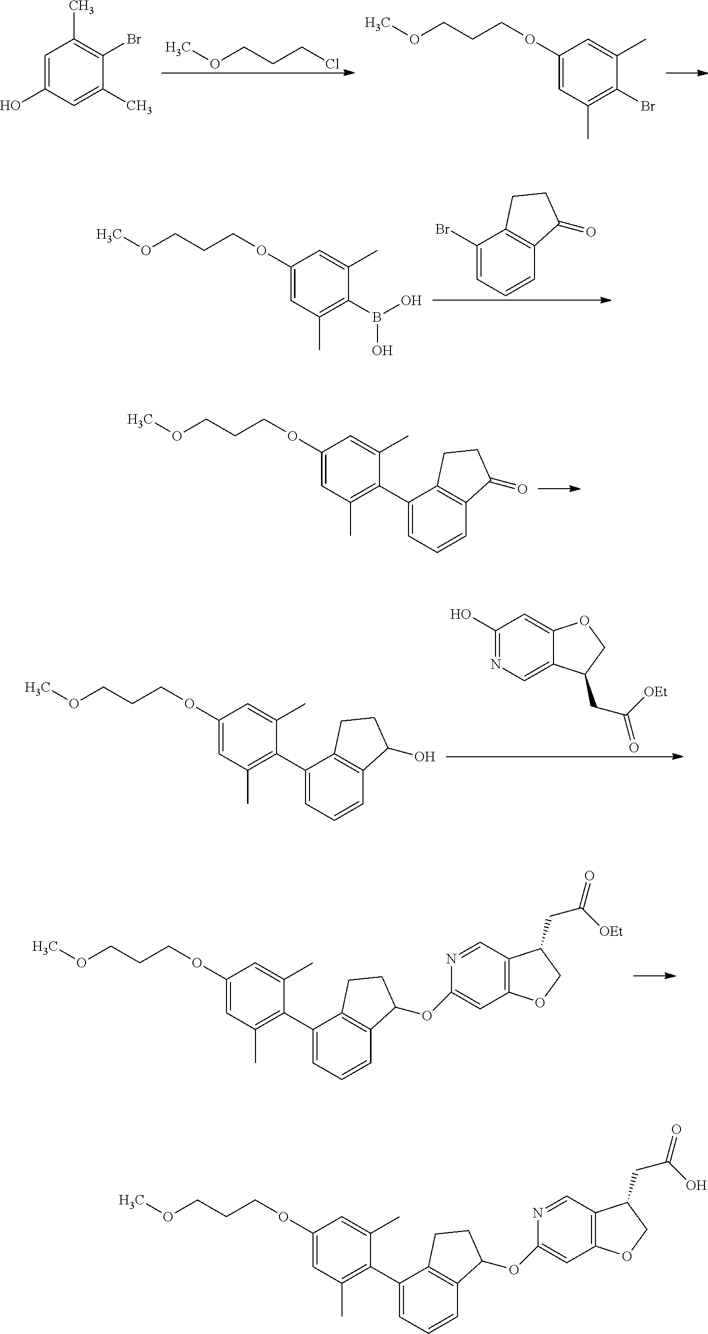

Under nitrogen atmosphere, hexane (50 mL) was added to sodium hydride (12.6 g, 264 mmol). The mixture was stirred for 30 sec, and stood still and the supernatant was removed. Tetrahydrofuran (460 mL) was added thereto, and the mixture was cooled to 0° C. A solution of 4-bromo-3,5-dimethylphenol (53.0 g, 264 mmol) in tetrahydrofuran (50 mL) was added slowly dropwise. After completion of the dropwise addition, and the mixture was stirred at 0° C. for 10 min, allowed to warm to room temperature, and was stirred for 20 min. Then 1-chloro-3-methoxypropane (30.1 g, 277 mmol) was added slowly at room temperature, and the mixture was stirred for 24 hr. The reaction mixture was diluted with 1 M aqueous sodium hydroxide solution (80 mL). Tetrahydrofuran was evaporated under reduced pressure, and the residue was extracted with diethyl ether. The extract was washed successively with 2M aqueous sodium hydroxide solution and saturated brine, dried over anhydrous magnesium sulfate, and concentrated under reduced pressure. The residue was purified by silica gel column chromatography (ethyl acetate:hexane=0:100-10:90) to give a colorless oil product 2-bromo-5-(3-methoxypropoxy)-1,3-dimethylbenzene (47.6 g, 66%). The subsequent synthetic procedure are similar to Example 3 was followed to arrive at the title compound, with the LC-MS[M+H]-m/z as 504.

### Example 7

### (S)-2-(6-((4′-(3-methoxypropoxy)-2′,6′-dimethyl-[1,1′-biphenyl]-3-yl)methoxy)-2,3-dihydrofuro[3,2-c]pyridin-3-yl)acetic acid

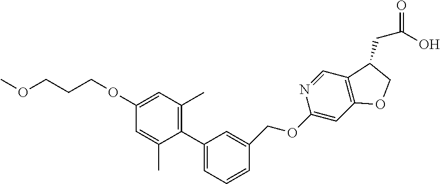 [SMILES: COCCCOc1cc(C)c(-c2cccc(COc3cc4c(cn3)[C@H](CC(=O)O)CO4)c2)c(C)c1]

Similar procedure to Example 4 was followed to arrive at the title compound, with the LC-MS[M+H]-m/z as 478.

### Example 8

### (S)-2-(6-((4′-((1,1-dioxidotetrahydro-2H-thiopyran-4-yl)oxy)-2′,6′-dimethyl-[1,1′-biphenyl]-3-yl)methoxy)-2,3-dihydrofuro[3,2-c]pyhdin-3-yl)acetic acid

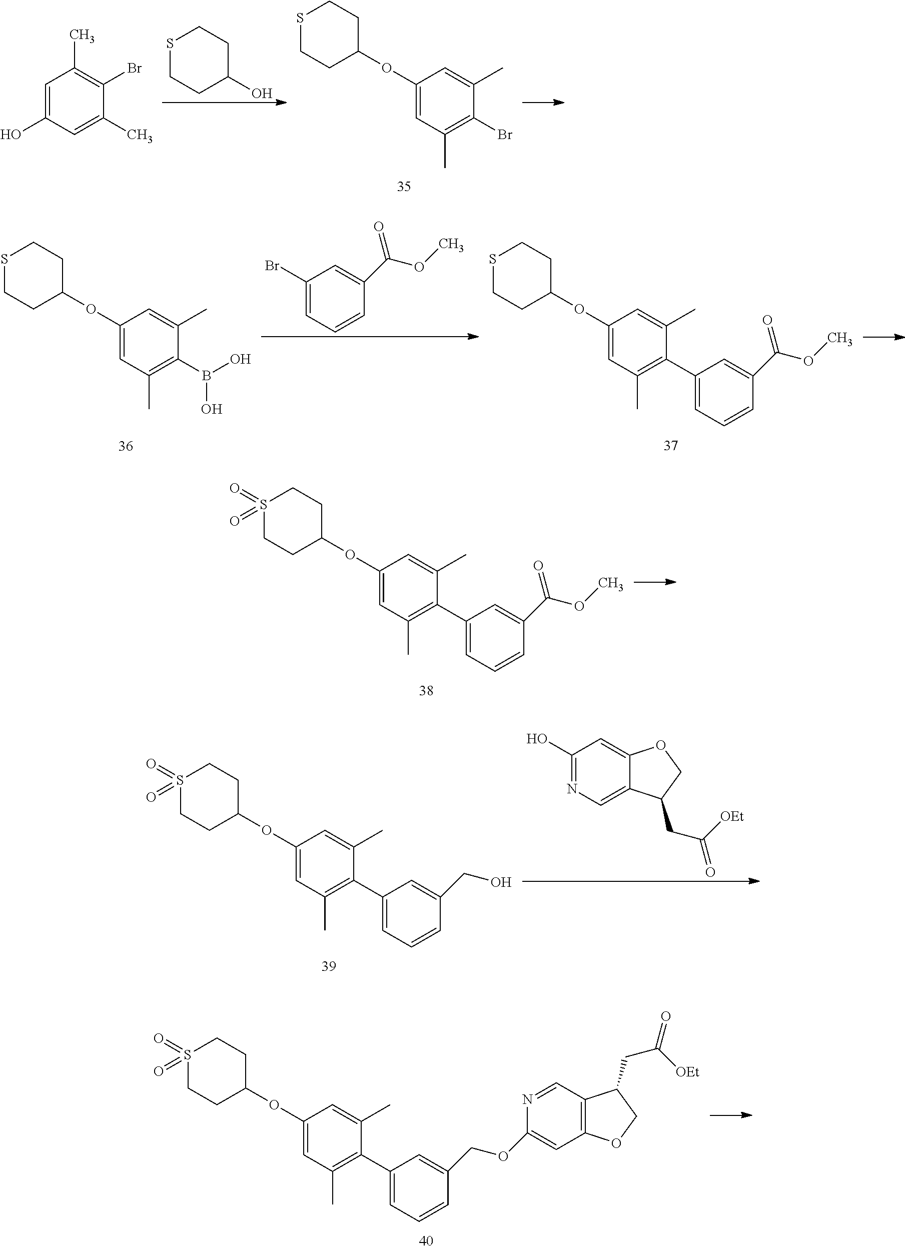

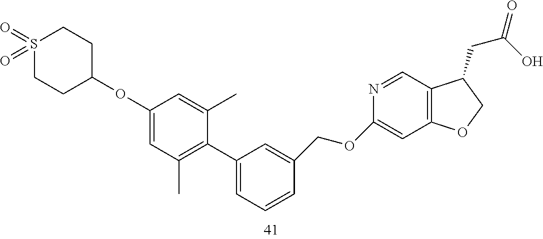 [SMILES: Cc1cc(OC2CCS(=O)(=O)CC2)cc(C)c1-c1cccc(COc2cc3c(cn2)[C@H](CC(=O)O)CO3)c1]

Step A: To a solution of 4-bromo-3,5-dimethylphenol (0.402 g, 2.00 mmol, tetrahydro-2H-thiopyran-4-ol (0.260 g, 2.20 mmol) and triphenylphosphine (0.682 g, 2.80 mmol) In tetrahydrofuran (10 mL) was added diethyl azodicarboxylate (40% solution in toluene, 1.18 mL, 2.60 mmol), and the mixture was stirred at room temperature for 1.5 hr. tetrahydro-2H-thiapyran-4-ol (0.118 g, 1.00 mmol), triphenylphosphine (0.314 g, 0.120 mmol) and diethyl azodicarboxylate (40% solution in toluene, 0.544 ml, 0.120 mmol) were added, and the mixture was further stirred for 1.5 hr. The reaction mixture was concentrated under reduced pressure, and the residue was purified by silica gel column chromatography (ethyl acetate:hexane=0:100-20:80) to give a colorless crystals product 35 (0.522 g, yield 87%).

Step B: To a solution of product 35 (3.01 g, 10.0 mmol) in tetrahydrofuran (50 mL) was added dropwise n-butyllithium hexane solution (1.8M, 6.57 mL, 10.5 mmol) at −78° C., and the reaction mixture was stirred for 1.5 hr at the same temperature. Triisopropyl borate (6.92 mL, 30.0 mmol) was added, and the mixture was stirred overnight, during which the mixture was allowed to warm to room temperature. The reaction mixture was ice-cooled, 2 M hydrochloric acid (50 mL) was added, and the mixture was stirred for 2.5 hr. The aqueous layer and the organic layer were separated, and the organic layer was washed with saturated brine and saturated aqueous sodium hydrogencarbonate while simultaneously adjusting to neutral. The organic layer was dried over anhydrous magnesium sulfate, and concentrated under reduced pressure. The residue was washed with cool hexane to give a colorless crystals product 36 (1.89 g, yield 71%).

Step C: Product 36 (30.5 mmol) and methyl 3-bromobenzoate (30.6 mmol) were dissolved in a mixture of 1M aqueous sodium carbonate solution (90 mL),

ethanol (30 mL) and toluene (90 mL). After argon substitution, tetrakis(triphenylphosphine)palladium(0) (1.77 g, 1.53 mmol) was added, and the reaction mixture was stirred at 80° C. for 24 hr under argon atmosphere. The reaction mixture was allowed to cook and water was added. The mixture was diluted with ethyl acetate, and the insoluble substrate was tittered off through celite. The organic layer of the filtrate was washed with saturated brine, dried over anhydrous magnesium sulfate, and concentrated under reduced pressure. The residue was purifiedby silica gel column chromatography (ethyl acetate:hexane=10:90-40:60) to give the pale-yellow compound 37 (5.72 g, yield 53%).

Step D: To a solution of product 37 (0.936 g, 2.63 mmol) in ethyl acetate (20 mL) was added m-chloroperbenzoic acid (65%, 1.46 g, 5.52 mmol) under ice-cooling, and the mixture was stirred for 16 hr, during which the mixture was allowed to gradually warm to room temperature. Ethyl acetate was added to the reaction mixture. The mixture was washed with a mixture of saturated aqueous sodium hydrogencarbonate and aqueous sodium thiosulfate solution, then washed with saturated brine, dried over anhydrous magnesium sulfate, and concentrated under reduced pressure. The residue was recrystallized from ethyl acetate-hexane to give the colorless crystals product 38 (0.870 g, yield 85%).

Step E: To a solution of product 38 (0.256 g, 0.66 mmol) in tetetrahydrofuran (5 mL) was added lithium aluminum hydride (80%, 31.4mg, 0.66 mmol) by small portions under ice-cooling, and the mixture was stirred at the same temperature for 1.5 hr. Sodium sulfate 10 hydrate (0.212 g, 0.66 mmol) was added by small portions to the reaction mixture, and the mixture was stirred at room temperature for 1 hr. The insoluble substance was filtered off through celite, and the filtrate was concentrated under reduced pressure to give the colorless product 39 (0.222 g, yield 93%).

The subsequent steps are similar procedure to step C and step D in Example 4 was followed to arrive at the title compound, with the LC-MS[M+H]-m/z as 538.

### Example 9

### 2-((3S)-6-4-(4-((1,1-dioxidotetrahydro-2H-thiopyran-4-yl)oxy)-2,6-dimethylphenyl)-2,3-dihydro-1H-inden-1-yl)oxy)-2,3-dihydrofuro[3,2-c]pyridin-3-yl)acetic acid

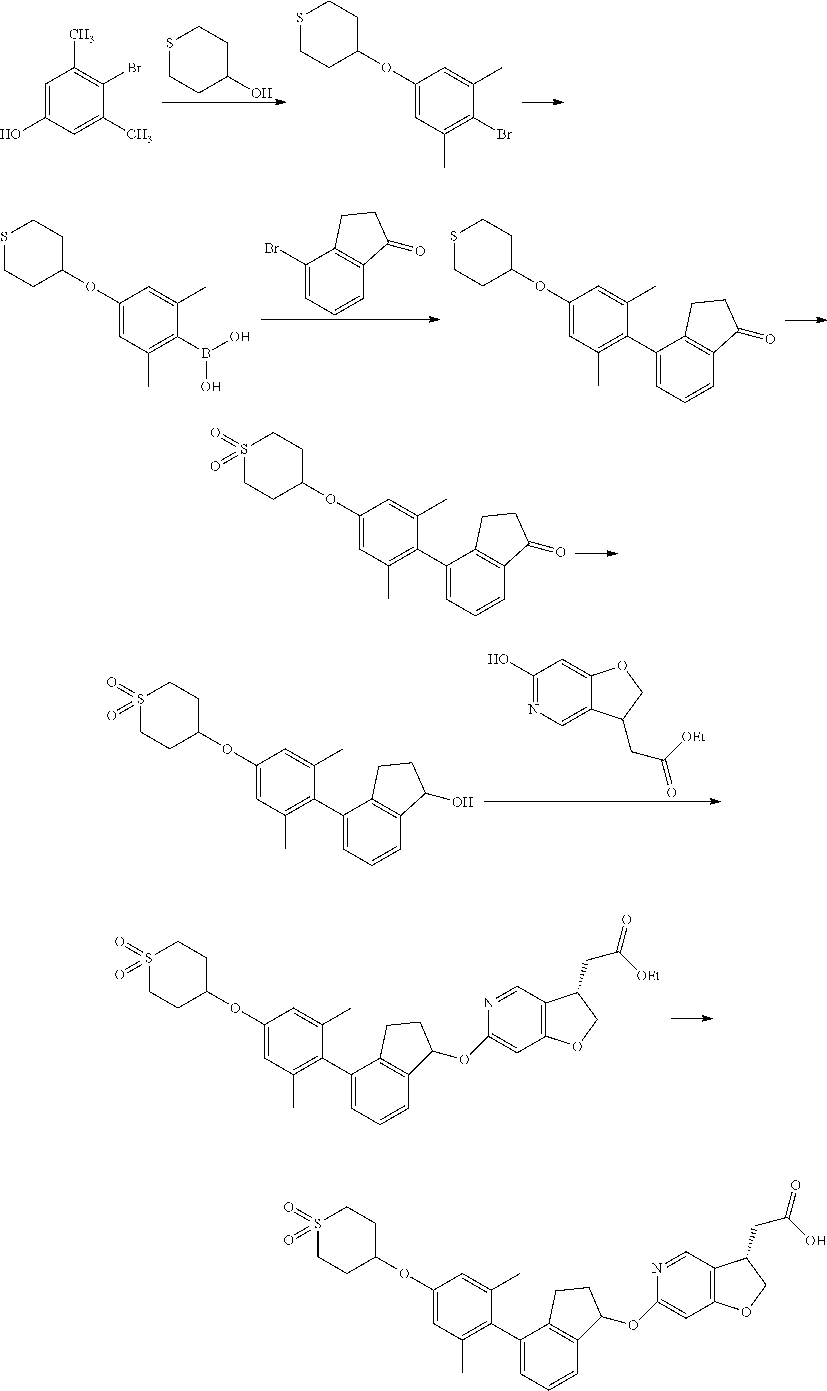

Similar procedure to Example 8 was followed to arrive at the title compound, with the LC-MS[M+H]-m/z as 564.

### Example 10

### 2-((S)-6-(((R)-4-(4-(3-methoxypropoxy)-2,6-dimethylphenyl)-2,3-dihydro-1H-inden-1-yl)oxy)-2,3-dihydrobenzo[b]thiophen-3-yl)acetic acid

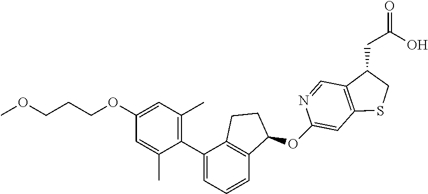 [SMILES: COCCCOc1cc(C)c(-c2cccc3c2CC[C@H]3Oc2cc3c(cn2)[C@H](CC(=O)O)CS3)c(C)c1]

Similar procedu re to Example 1 was followed to arrive at the title compound, with the LC-MS[M+H]-m/z as 519.

### Example 11

### Ethyl 2-((S)-6-(((S)-4-(4-(3-methoxypropoxy)-2,6-dimethylphenyl)-2,3-dihydro-1H-inden-1-yl)oxy)-2,3-dihydrobenzo[b]thiophen-3-yl)acetate

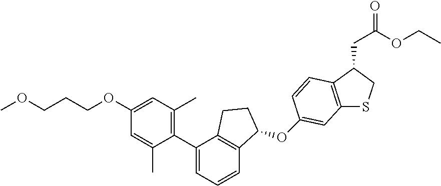 [SMILES: CCOC(=O)C[C@@H]1CSc2cc(O[C@H]3CCc4c(-c5c(C)cc(OCCCOC)cc5C)cccc43)ccc21]

Similar procedure to Example 1 was followed to arrive at the title compound, with the LC-MS[M+H]-m/z as 547.

### Example 12

### 2-((S)-6-(((S)-4-(2,6-dimethyl-4-(3-(methylsulfonyl)propoxy)phenyl)-2,3-dihydro-1H-inden-1-yl)oxy)-2,3-dihydrobenzo[b]thiophen-3-yl)acetic acid

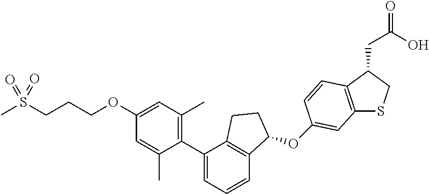 [SMILES: Cc1cc(OCCCS(C)(=O)=O)cc(C)c1-c1cccc2c1CC[C@@H]2Oc1ccc2c(c1)SC[C@H]2CC(=O)O]

Similar procedure to Example 1 was followed to arrive at the title compound, with the LC-MS[M+H]-m/z as 567.

### Example 13

### 2-((S)-6-(((R)-4-(2,6-dimethyl-4-(3-(methylsulfonyl)propoxy)phenyl)-2,3-dihydro-1H-inden-1-yl)oxy)-2,3-dihydrobenzo[b]thiophen-3-yl)acetic acid

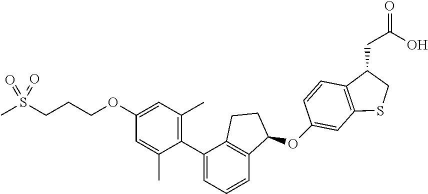 [SMILES: Cc1cc(OCCCS(C)(=O)=O)cc(C)c1-c1cccc2c1CC[C@H]2Oc1ccc2c(c1)SC[C@H]2CC(=O)O]

Similar procedure to Example 1 was followed to arrive at the title compound, with the LC-MS[M+H]-m/z as 567.

### Example 14

### (2-((S)-6-(((R)-4-(2,6-dimethyl-4-(3-(methylsulfonyl)propoxy)phenyl)-2,3-dihydro-1H-inden-1-yl)oxy)-2,3-dihydrobenzo[b]thiophen-3-yl)acetoxy)methyl pivalate

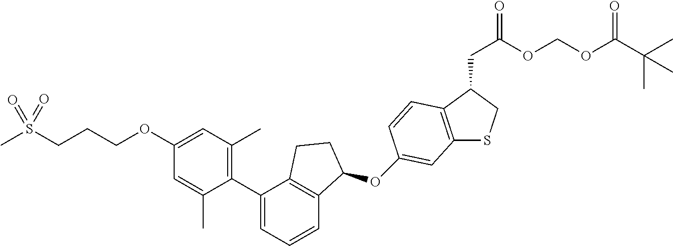 [SMILES: Cc1cc(OCCCS(C)(=O)=O)cc(C)c1-c1cccc2c1CC[C@H]2Oc1ccc2c(c1)SC[C@H]2CC(=O)OCOC(=O)C(C)(C)C]

A solution of 2-((S)-6-(((R)-4-(2,6-dimethyl-4-(3-(methylsulfonyl)propoxy)phenyl)-2,3-dihydro-1H-inden-1-yl)oxy)-2,3-dihydrobenzo[b]thiophen-3-yl)acetic acid (0.56 g, 1 mmol) in DMAc (15 ml) was treated with sodium carbonate (0.127 g, 1.2 mmol) at −5° C. for 10 min under a N₂atmosphere. The mixture was cooled to −15° C., followed by addition of iodomethyl pivalate (0.29 g, 1.2 mmol). The mixture was stirred vigorously at −15° C. for 1 hr. The reaction mixture was added under vigorous stirring to a mixture of ethyl acetate (30 ml) and water (40 mL). The organic layer was separated and washed with brine solution (2×30 mL), dried over anhydrous magnesium sulfate, and concentrated under reduced pressure. The residue was purified by silica gel column chromatography to give the title compound, with the LC-MS[M+H]-m/z as 681.

### Example 15

### (2-((S)-6-(((R)-4-(4-(3-methoxyporpoxy)-2,6-dimethylphenyl)-2,3-dihydro-1H-inden-1-yl)oxy)-2,3-dihydrobenzo[b]thiophen-3-yl)acetoxy)methyl isobutyrate

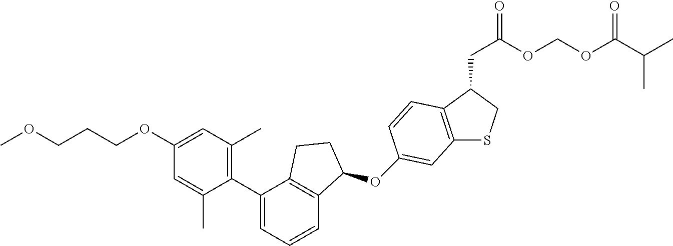 [SMILES: COCCCOc1cc(C)c(-c2cccc3c2CC[C@H]3Oc2ccc3c(c2)SC[C@H]3CC(=O)OCOC(=O)C(C)C)c(C)c1]

Similar procedure to Example 14 was followed to arrive at the title compound, with the LC-MS[M+H]-m/z as 619.

### Example 16

### 2-((S)-6-(((S)-4-(2,6-dimethyl-4-(3-(methylsulfonyl)propoxy)phenyl)-2,3-dihydro-1H-inden-1-yl)oxy)-2,3-dihydrofuro[3,2-c]pyridin-3-yl)acetic acid

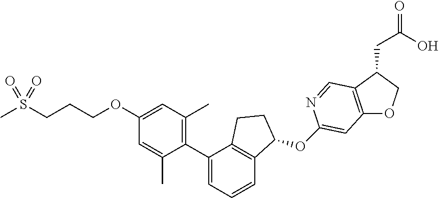 [SMILES: Cc1cc(OCCCS(C)(=O)=O)cc(C)c1-c1cccc2c1CC[C@@H]2Oc1cc2c(cn1)[C@H](CC(=O)O)CO2]

Similar procedure to Example 1 was followed to arrive at the title compound, with the LC-MS[M+H]-m/z as 552.

### Example 17

### 2-((S)-6-(((R)-4-(2,6-dimethyl-4-(3-(methylsulfonyl)propoxy)phenyl)-2,3-dihydro-1H-inden-1-yl)oxy)-2,3-dihydrofuro[3,2-c]pyridin-3-yl)acetic acid

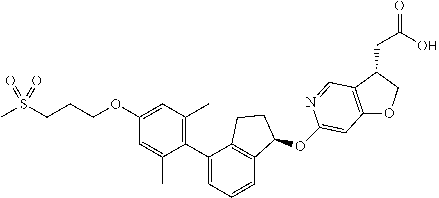 [SMILES: Cc1cc(OCCCS(C)(=O)=O)cc(C)c1-c1cccc2c1CC[C@H]2Oc1cc2c(cn1)[C@H](CC(=O)O)CO2]

Similar procedure to Example 1 was followed to arrive at the title compound, with the LC-MS[M+H]-m/z as 552.

### Example 18

### 2-((3S)-6-((4-(2,6-dimethylphenyl)-2,3-dihydro-1H-inden-1-yl)oxy)-2,3-dihydrofuro[3,2-c]pyridin-3-yl)acetic acid

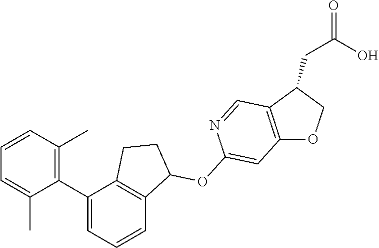 [SMILES: Cc1cccc(C)c1-c1cccc2c1CCC2Oc1cc2c(cn1)[C@H](CC(=O)O)CO2]

Similar procedure to Example 3 was followed to arrive at the title compound, with the LC-MS[M+H]-m/z as 416.

### Example 19

### (S)-2-(6-((2′,6′-dimethyl-[1,1′-biphenyl]-3-yl)methoxy)-2,3-dihydrofuro[3,2-c]pyridin-3-yl)acetic acid

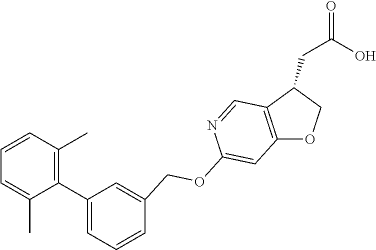 [SMILES: Cc1cccc(C)c1-c1cccc(COc2cc3c(cn2)[C@H](CC(=O)O)CO3)c1]

Similar procedure to Example 2 was followed to arrive at the title compound, with the LC-MS[M+H]-m/z as 390.

### Example 20

### 2-((3S)-6-((4-(4,6-dimethylpyrimidin-5-yl)-2,3-dihydro-1H-inden-1-yl)oxy)-2,3-dihydrofuro[3,2-c]pyridin-3-yl)acetic acid

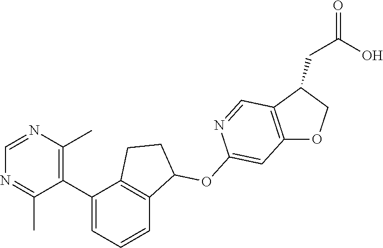 [SMILES: Cc1ncnc(C)c1-c1cccc2c1CCC2Oc1cc2c(cn1)[C@H](CC(=O)O)CO2]

Similar procedure to Example 3 was followed to arrive at the title compound, with the LC-MS[M+H]-m/z as 418.

### Example 21

### (S)-2-(6-((3-(4,6-dimethylpyrimidin-5-yl)benzyl)oxy)-2,3-dihydrofuro[3,2-c]pyridin-3-yl)acetic acid

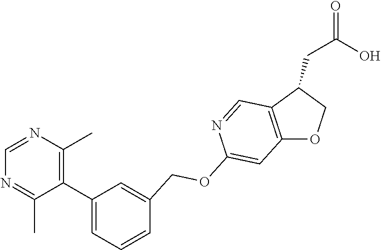 [SMILES: Cc1ncnc(C)c1-c1cccc(COc2cc3c(cn2)[C@H](CC(=O)O)CO3)c1]

Similar procedure to Example 2 was followed to arrive at the title compound, with the LC-MS[M+H]-m/z as 392.

### Example 22

### 2-((3S)-6-((4-(2,4-dimethylpyridin-3-yl)-2,3-dihydro-1H-inden-1-yl)oxy)-2,3-dihydrofuro[3,2-c]pyridin-3-yl)acetic acid

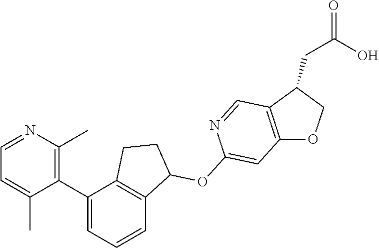 [SMILES: Cc1ccnc(C)c1-c1cccc2c1CCC2Oc1cc2c(cn1)[C@H](CC(=O)O)CO2]

Similar procedure to Example 3 was followed to arrive at the title compound, with the LC-MS[M+H]-m/z as 417.

### Example 23

### (S)-2-(6-((3-(2,4-dimethylpyridin-3-yl)benzyl)oxy)-2,3-dihydrofuro[3,2-c]pyridin-3-yl)acetic acid

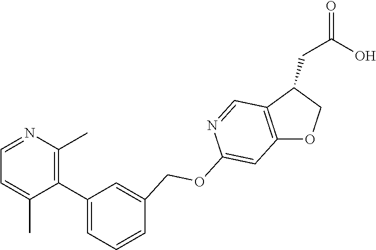 [SMILES: Cc1ccnc(C)c1-c1cccc(COc2cc3c(cn2)[C@H](CC(=O)O)CO3)c1]

Similar procedure to Example 2 was followed to arrive at the title compound, with the LC-MS[M+H]-m/z as 391.

### Example 24

### 2-((3S)-6-((4-molpholino-2,3-dihydro-1H-inden-1-yl)oxy)-2,3-dihydrofuro[3,2-c]pyridin-3-yl)acetic acid

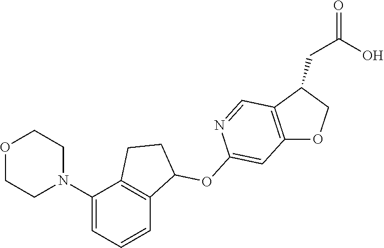 [SMILES: O=C(O)C[C@@H]1COc2cc(OC3CCc4c3cccc4N3CCOCC3)ncc21]

Similar procedure to Example 3 was followed to arrive at the title compound, with the LC-MS[M+H]-m/z as 397.

### Example 25

### (S)-2-(6-((3-morpholinobenzyl)oxy)-2,3-dihydrofuro[3,2-c]pyridin-3-yl)acetic acid

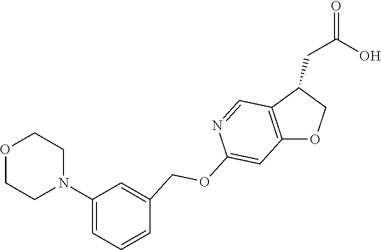 [SMILES: O=C(O)C[C@@H]1COc2cc(OCc3cccc(N4CCOCC4)c3)ncc21]

Similar procedure to Example 2 was followed to arrive at the title compound, with the LC-MS[M+H]-m/z as 371.

### Example 26

### 2-((3S)-6-((4-(1-methyl-1H-imidazol-2-yl)-2,3-dihydro-1H-inden-1-yl)oxy)-2,3-dihydrofu ro[3,2-c]pyridin-3-yl)acetic acid

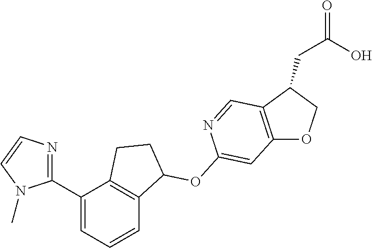 [SMILES: Cn1ccnc1-c1cccc2c1CCC2Oc1cc2c(cn1)[C@H](CC(=O)O)CO2]

Similar procedure to Example 3 was followed to arrive at the title compound, with the LC-MS[M+H]-m/z as 392.

### Example 27

### (S)-2-(6-((3-(1-methyl-1H-imidazol-2-yl)benzyl)oxy)-2,3-dihydrofuro[3,2-c]pyridin-3-yl)acetic acid

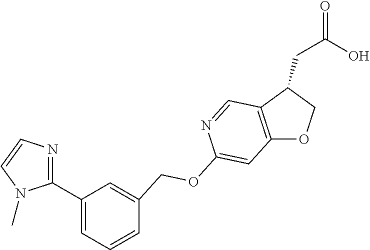 [SMILES: Cn1ccnc1-c1cccc(COc2cc3c(cn2)[C@H](CC(=O)O)CO3)c1]

Similar procedure to Example 2 was followed to arrive at the title compound, with the LC-MS[M+H]-m/z as 366.

### Example 28

### 2-((3S)-6-((4-(2,6-dimethylphenyl)-2,3-dihydro-1H-inden-1-yl)oxy)-2,3-dihydrothieno[3,2-c]pyridin-3-yl)acetic acid

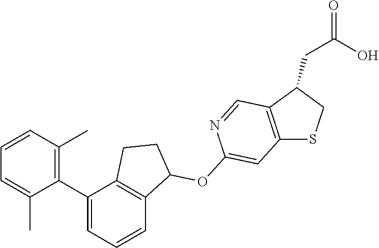 [SMILES: Cc1cccc(C)c1-c1cccc2c1CCC2Oc1cc2c(cn1)[C@H](CC(=O)O)CS2]

Similar procedure to Example 3 was followed to arrive at the title compound, with the LC-MS[M+H]-m/z as 432.

### Example 29

### (S)-2-(6-((2′,6′-dimethyl-[1,1′-biphenyl]-3-yl)methoxy)-2,3-dihydrothieno[3,2-c]pyridin-3-yl)acetic acid

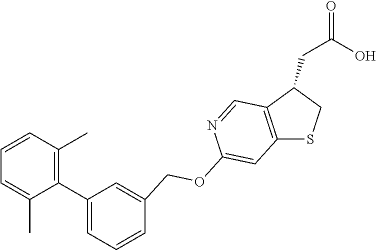 [SMILES: Cc1cccc(C)c1-c1cccc(COc2cc3c(cn2)[C@H](CC(=O)O)CS3)c1]

Similar procedure to Example 2 was followed to arrive at the title compound, with the LC-MS[M+H]-m/z as 406.

### Example 30

### 2-((3S)-6-((4-(4,6-dimethylpyrimidin-5-yl)-2,3-dihydro-1H-inden-1-yl)oxy)-2,3-dihydrothieno[3,2-c]pyridin-3-yl)acetic acid

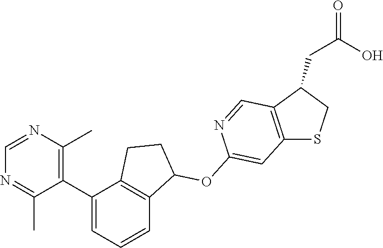 [SMILES: Cc1ncnc(C)c1-c1cccc2c1CCC2Oc1cc2c(cn1)[C@H](CC(=O)O)CS2]

Similar procedure to Example 3 was followed to arrive at the title compound, with the LC-MS[M+H]-m/z as 434.

### Example 31

### (S)-2-(6-((3-(4,6di,ethylpyrimidin-5_yl)benzyl)oxy)-2,3-dihydrothieno[3,2-c]pyridin-3-yl)acetic acid

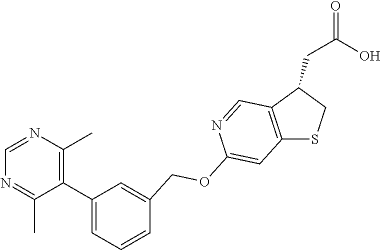 [SMILES: Cc1ncnc(C)c1-c1cccc(COc2cc3c(cn2)[C@H](CC(=O)O)CS3)c1]

Similar procedure to Example 2 was followed to arrive at the title compound, with the LC-MS[M+H]-m/z as 408.

### Example 32

### 2-((3S)-6-((4-(2,4-dimethylpyridin-3-yl)-2,3-dihydro-1H-inden-1-yl)oxy)-2,3-dihydrothieno[3,2-c]pyridin-3-yl)acetic acid

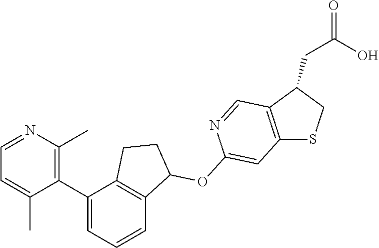 [SMILES: Cc1ccnc(C)c1-c1cccc2c1CCC2Oc1cc2c(cn1)[C@H](CC(=O)O)CS2]

Similar procedure to Example 3 was followed to arrive at the title compound, with the LC-MS[M+H]-m/z as 433.

### Example 33

### (S)-2-(6-((3-(2,4-dimethylpyridin-3-yl)benzyl)oxy)-2,3-dihydrothieno[3,2-c]pyridin-3-yl)acetic acid

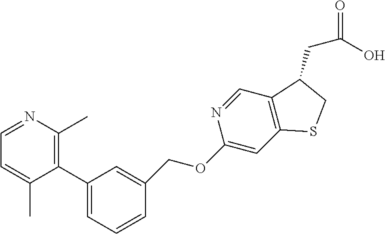 [SMILES: Cc1ccnc(C)c1-c1cccc(COc2cc3c(cn2)[C@H](CC(=O)O)CS3)c1]

Similar procedure to Example 2 was followed to arrive at the title compound, with the LC-MS[M+H]-m/z as 407.

### Example 34

### 2-((3S)-6-((4-morpholino-2,3-dihydro-1H-inden-1-yl)oxy)-2,3-dihydrothieno[3,2-c]pyridin-3-yl)acetic acid

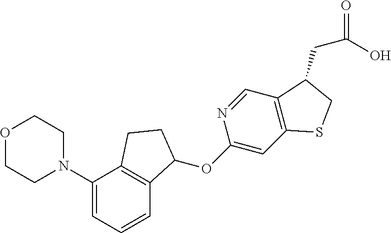 [SMILES: O=C(O)C[C@@H]1CSc2cc(OC3CCc4c3cccc4N3CCOCC3)ncc21]

Similar procedure to Example 3 was followed to arrive at the title compound, with the LC-MS[M+H]-m/z as 413.

### Example 35

### (S)-2-(6-((3-morpholinobenzyl)oxy)-2,3-dihydrothieno[3,2-c]pyridin-3-yl)acetic acid

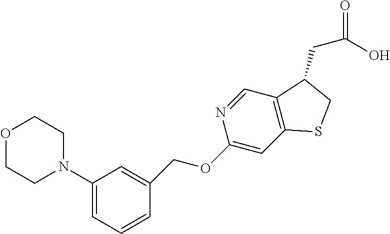 [SMILES: O=C(O)C[C@@H]1CSc2cc(OCc3cccc(N4CCOCC4)c3)ncc21]

Similar procedure to Example 2 was followed to arrive at the title compound, with the LC-MS[M+H]-m/z as 387.

### Example 36

### 2-((3S)-6-((4-(1-methyl-1H-imidazol-2-yl)-2,3-dihydro-1H-inden-1-yl)oxy)-2,3dihydrothieno[3,2-c]pyridin-3yl)acetic acid

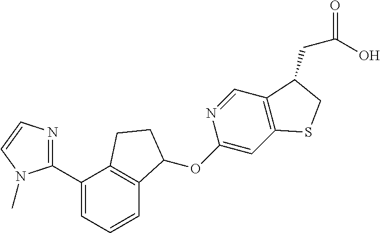 [SMILES: Cn1ccnc1-c1cccc2c1CCC2Oc1cc2c(cn1)[C@H](CC(=O)O)CS2]

Similar procedure to Example 3 was followed to arrive at the title compound, with the LC-MS[M+H]-m/z as 408.

### Example 37

### (S)-2-(6-((3-(1-methyl-1H-imidazol-2-yl)benzyl)oxy)-2,3-dihydrothieno[3,2-c]pyridin-3-yl)acetic acid

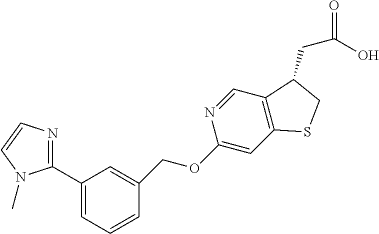 [SMILES: Cn1ccnc1-c1cccc(COc2cc3c(cn2)[C@H](CC(=O)O)CS3)c1]

Similar procedure to Example 2 was followed to arrive at the title compound, with the LC-MS[M+H]-m/z as 382.

### Example 38

### 2-((3S)-6-((4-(2,6-dimethyl-4-(3-(methylsulfonyl)propoxy)phenyl)-2,3-dihydro-1H-inden-1-yl)oxy)-2,3-dihydrothieno[3,2-c]pyridin-3-yl)acetic acid

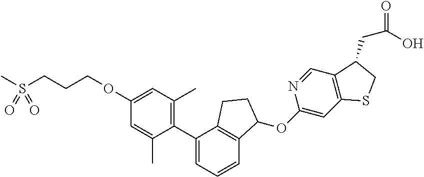 [SMILES: Cc1cc(OCCCS(C)(=O)=O)cc(C)c1-c1cccc2c1CCC2Oc1cc2c(cn1)[C@H](CC(=O)O)CS2]

Similar procedu re to Example 3 was followed to arrive at the title compound, with the LC-MS[M+H]-m/z as 568.

### Example 39

### (S)-2-(6-((2′,6′-dimethyl-4′-(3-(methylsulfonyl)propoxy)[1,1′-biphenyl]-3-yl)methoxy)-2,3-dihydrothieno[3,2-c]pyridin-3-yl)acetic acid

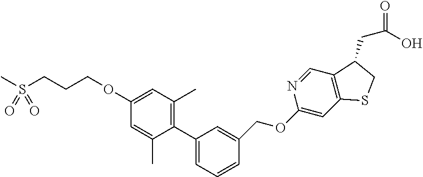 [SMILES: Cc1cc(OCCCS(C)(=O)=O)cc(C)c1-c1cccc(COc2cc3c(cn2)[C@H](CC(=O)O)CS3)c1]

Similar procedure to Example 2 was followed to arrive at the title compound, with the LC-MS[M+H]-m/z as 542.

### Example 40

### 2-((3S)-6-((4-(2,6-dimethyl-4-(3-(methylthio)propoxy)phenyl)-2,3-dihydro-1H-inden-1-yl)oxy)-2,3-dihydrothieno[3,2-c]pyridin-3-yl)acetic acid

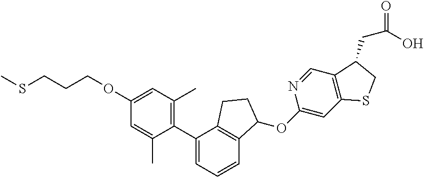 [SMILES: CSCCCOc1cc(C)c(-c2cccc3c2CCC3Oc2cc3c(cn2)[C@H](CC(=O)O)CS3)c(C)c1]

Similar procedure to Example 5 was followed to arrive at the title compound, with the LC-MS[M+H]-m/z as 536.

### Example 41

### (S)-2-(6-((2′,6′-dimethyl-4′-(3-(methylthio)propoxy)-[1,1′-biphenyl]-3-yl)methoxy)-2,3-dihydrothieno[3,2-c]pyridin-3-yl)acetic acid

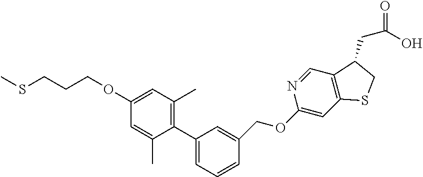 [SMILES: CSCCCOc1cc(C)c(-c2cccc(COc3cc4c(cn3)[C@H](CC(=O)O)CS4)c2)c(C)c1]

Similar procedure to Example 4 was followed to arrive at the title compound, with the LC-MS[M+H]-m/z as 510.

### Example 42

### 2-((3S)-6-((4-(4-(3-methoxypropoxy)-2,6-dimethylphenyl)-2,3-dihydro-1H-inden-1-yl)oxy)-2,3-dihydrothieno[3,2-c]pyridin-3-yl)acetic acid

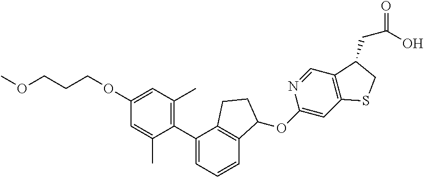 [SMILES: COCCCOc1cc(C)c(-c2cccc3c2CCC3Oc2cc3c(cn2)[C@H](CC(=O)O)CS3)c(C)c1]

Similar procedure to Example 6 was followed to arrive at the title compound, with the LC-MS[M+H]-m/z as 520.

### Example 43

### (S)-2-(6-((4′-(3-methoxypropoxy)-2′,6′-dimethyl-[1,1′-biphenyl]-3-yl)methoxy)-2,3-dihydrothieno[3,2-c]pyridin-3-yl)acetic acid

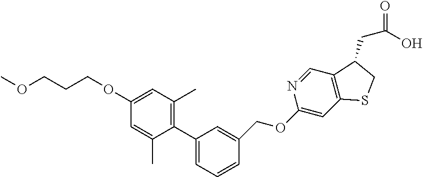 [SMILES: COCCCOc1cc(C)c(-c2cccc(COc3cc4c(cn3)[C@H](CC(=O)O)CS4)c2)c(C)c1]

Similar procedure to Example 6 was followed to arrive at the title compound, with the LC-MS[M+H]-m/z as 494.

### Example 44

### 2-((3S)-6-((4-(4-(1,1-dioxidotetrahydro-2H-thiopyran-4-yl)oxy)2,6-dimethylphenyl)-2,3-dihydro-1H-inden-1-yl)oxy)-2,3-dihydrothieno[3,2-c]pyridin-3-yl)acetic acid

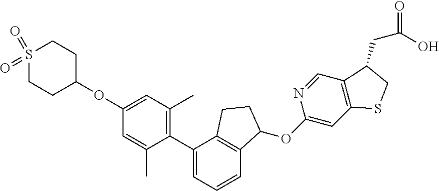 [SMILES: Cc1cc(OC2CCS(=O)(=O)CC2)cc(C)c1-c1cccc2c1CCC2Oc1cc2c(cn1)[C@H](CC(=O)O)CS2]

Similar procedure to Example 8 and Example 3 was followed to arrive at the title compound, with the LC-MS[M+H]-m/z as 580.

### Example 45

### (S)-2-(6-((4′-((1,1-dioxidotetrahydro-2H-thiopyran-4-yl)oxy)-2′,6′-dimethyl-[1,1′-biphenyl]-3-yl)methoxy)-2,3-dihydrothieno[3,2-c]pyridin-3-yl)acetic acid

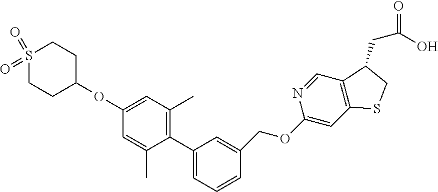 [SMILES: Cc1cc(OC2CCS(=O)(=O)CC2)cc(C)c1-c1cccc(COc2cc3c(cn2)[C@H](CC(=O)O)CS3)c1]

Similar procedure to Example 8 was followed to arrive at the title compound, with the LC-MS[M+H]-m/z as 554.

### Example 46

### 2-((3S)-6-((4-(2,6-dimethylphenyl)-2,3-dihydro-1H-inden-1-yl)oxy)-2,3-dihydrobenzo[b]thiophen-3-yl)acetic acid

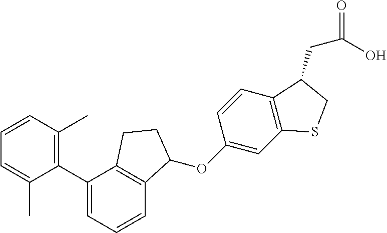 [SMILES: Cc1cccc(C)c1-c1cccc2c1CCC2Oc1ccc2c(c1)SC[C@H]2CC(=O)O]

Similar procedure to Example 3 was followed to arrive at the title compound, with the LC-MS[M+H]-m/z as 431.

### Example 47

### 2-(3S)-6-((4-(4,6-dimethylpyrimidin-5-yl)-2,3-dihydro-1H-indin-1-yl)oxy)-2,3-dihydrobenzo[b]thiophen-3-yl)acetic acid

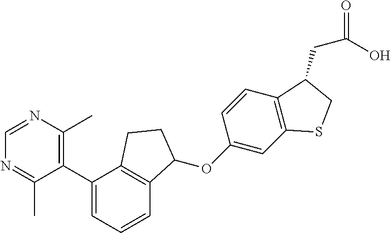 [SMILES: Cc1ncnc(C)c1-c1cccc2c1CCC2Oc1ccc2c(c1)SC[C@H]2CC(=O)O]

Similar procedure to Example 3 was followed to arrive at the title compound, with the LC-MS[M+H]-m/z as 433.

### Example 48

### 2-((3S)-6-((4-(2,4-dimethylpyridin-3-yl)-2,3-dihydro-1H-inden-1-yl)oxy)-2,3-dihydrobenzo[b]thiophen-3-yl)acetic acid

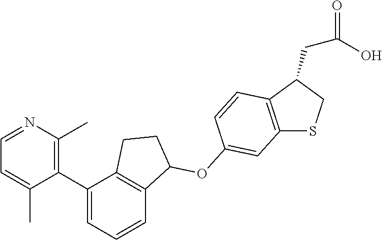 [SMILES: Cc1ccnc(C)c1-c1cccc2c1CCC2Oc1ccc2c(c1)SC[C@H]2CC(=O)O]

Similar procedure to Example 3 was followed to arrive at the title compound, with the LC-MS[M+H]-m/z as 432.

### Example 49

### 2-((3S)-6-((4-morpholino-2,3-dihydro-1H-inden-1-yl)oxy)-2,3-dihydrobenzo[b]thiophen-3-yl)acetic acid

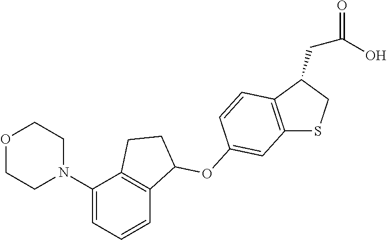 [SMILES: O=C(O)C[C@@H]1CSc2cc(OC3CCc4c3cccc4N3CCOCC3)ccc21]

Similar procedure to Example 3 was followed to arrive at the title compound, with the LC-MS[M+H]-m/z as 412.

### Example 50

### 2-((3S)-6-((4-(1-methyl-1H-imidazol-2-yl)-2,3-dihydro-1H-inden-1-yl)oxy)-2,3-dihydrobenzo[b]thiophen-3-yl)acetic acid

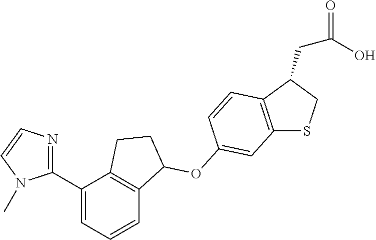 [SMILES: Cn1ccnc1-c1cccc2c1CCC2Oc1ccc2c(c1)SC[C@H]2CC(=O)O]

Similar procedure to Example 3 was followed to arrive at the title compound, with the LC-MS[M+H]-m/z as 407.

### Example 51

### 2-((3S)-6-((4-(2,6-dimethyl-4-(3-(methylsulfonyl)propoxy)phenyl)-2,3-dihydro-1H-inden-1-yl)oxy)-2,3-dihydrobenzo[b]thiophen-3-yl)acetic acid

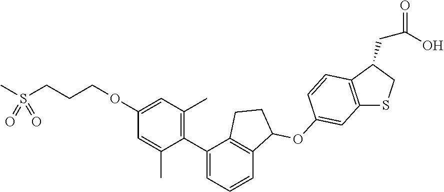 [SMILES: Cc1cc(OCCCS(C)(=O)=O)cc(C)c1-c1cccc2c1CCC2Oc1ccc2c(c1)SC[C@H]2CC(=O)O]

Similar procedure to Example 3 was followed to arrive at the title compound, with the LC-MS[M+H]-m/z as 567.

### Example 52

### 2-((3S)-6-((4-(2,6-dimethyl-4-(3-(metylthio)porpoxy)phenyl)-2,3-dihydro-1H-inden-1-yl)oxy)-2,3-dihydrobenzo[b]thiophen-3-yl)acetic acid

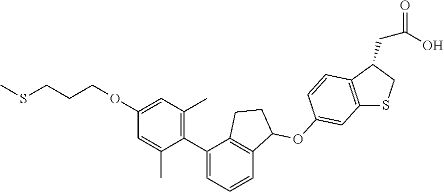 [SMILES: CSCCCOc1cc(C)c(-c2cccc3c2CCC3Oc2ccc3c(c2)SC[C@H]3CC(=O)O)c(C)c1]

Similar procedure to Example 5 was followed to arrive at the title compound, with the LC-MS[M+H]-m/z as 535.

### Example 53

### 2-((3S)-6-((4-(4-(3-methoxypropoxy)-2,6-dlmethylphenyl)-2,3-dihydro-1H-inden-1-yl)oxy)-2,3-dihydrobenzo[b]thiophen-3-yl)acetic acid

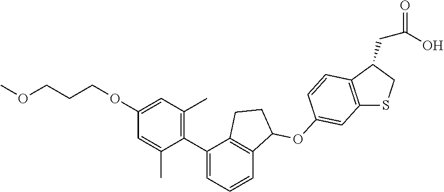 [SMILES: COCCCOc1cc(C)c(-c2cccc3c2CCC3Oc2ccc3c(c2)SC[C@H]3CC(=O)O)c(C)c1]

Similar procedure to Example 6 was followed to arrive at the title compound, with the LC-MS[M+H]-m/z as 519.

### Example 54

### 2-((3S)-6-((4-(4-((1,1-dioxidotetrahydro-2H-thiopyran-4-yl)oxy)-2,6-dimethylphenyl)-2,3-dihydro-1H-inden-1-yl)oxy)-2,3-dihydrobenzo[b]thiophen-3-yl)acetic acid

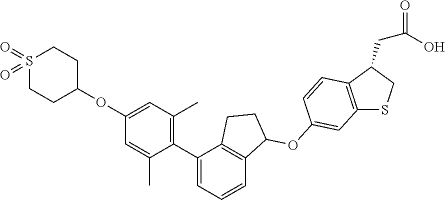 [SMILES: Cc1cc(OC2CCS(=O)(=O)CC2)cc(C)c1-c1cccc2c1CCC2Oc1ccc2c(c1)SC[C@H]2CC(=O)O]

Similar procedure to Example 8 and Example 3 was followed to arrive at

the title compound, with the LC-MS[M+H]-m/z as 579.

### Example 55

### 2-((3S)-6-((4-mesityl-2,3-dihydro-1H-inden-1-yl)oxy)-7-methyl-2,3-dihydrofuro[3,2-c]pyridin-3-yl)acetic acid

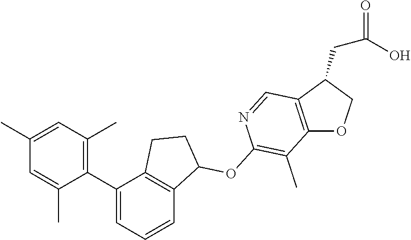 [SMILES: Cc1cc(C)c(-c2cccc3c2CCC3Oc2ncc3c(c2C)OC[C@H]3CC(=O)O)c(C)c1]

Similar procedure to Example 3 was followed to arrive at the title compound, with the LC-MS[M+H]-m/z as 444.

### Example 56

### 2-((3S)-6-((4-(4-methoxy-2,6-dimethylphenyl)-2,3-dihydro-1H-inden-1-yl)oxy)-2,3-dihydrofuro[3,2-c]pyridin-3-yl)acetic acid

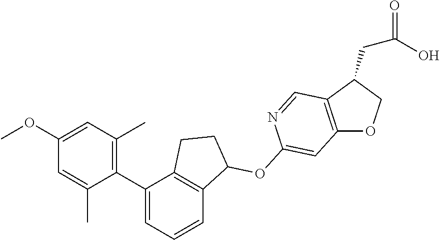 [SMILES: COc1cc(C)c(-c2cccc3c2CCC3Oc2cc3c(cn2)[C@H](CC(=O)O)CO3)c(C)c1]

Similar procedure to Example 3 was followed to arrive at the title compound, with the LC-MS[M+H]-m/z as 446.

### Example 57

### 2-((3S)-7-bromo-6-((4-(2,6-dimethyl-4-(trifluoromethyl)phenyl)-2,3-dihydro-1H-inden-1-yl)oxy)-2,3-dihydrofuro[3,2-c]pyridin-3-yl)acetic acid

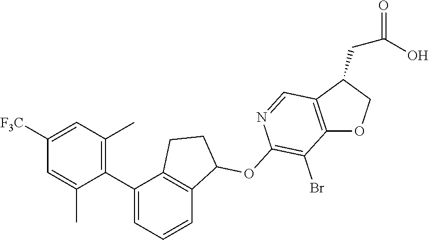 [SMILES: Cc1cc(C(F)(F)F)cc(C)c1-c1cccc2c1CCC2Oc1ncc2c(c1Br)OC[C@H]2CC(=O)O]

Similar procedure to Example 3 was followed to arrive at the title compound, with the LC-MS[M+H]-m/z as 563.

### Example 58

### 2-((3S)-6-((4-(2,6-dimethyl-4-(trifluoromethoxy)phenyl)-2,3-dihydro-1H-inden-1-yl)oxy)-2,3-dihydrofuro[3,2-c]pyridin-3-yl)acetic acid

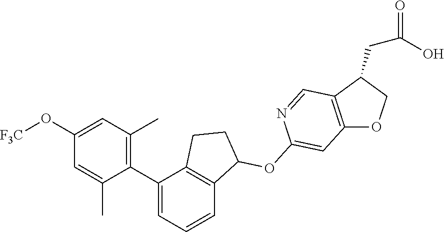 [SMILES: Cc1cc(OC(F)(F)F)cc(C)c1-c1cccc2c1CCC2Oc1cc2c(cn1)[C@H](CC(=O)O)CO2]

Similar procedure to Example 3 was followed to arrive at the title compound, with the LC-MS[M+H]-m/z as 500.

### Example 59

### 2-((3S)-6-((4-(4-cyclohexyl-2,6-dimethylphenyl)-6-methyl-2,3-dihydro-1H-inden-1-yl)oxy)-2,3-dihydrofuro[3,2-c]pyridin-3-yl)acetic acid

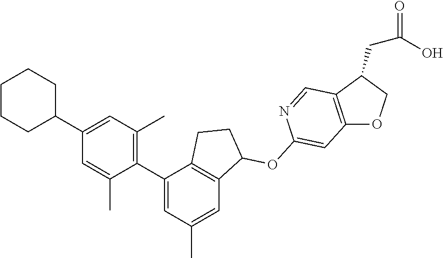 [SMILES: Cc1cc(-c2c(C)cc(C3CCCCC3)cc2C)c2c(c1)C(Oc1cc3c(cn1)[C@H](CC(=O)O)CO3)CC2]

Similar procedure to Example 3 was followed to arrive at the title compound, with the LC-MS[M+H]-m/z as 512.

### Example 60

### 2-((3S)-6-((4-(2,6-dimethyl-4-(3-(methylsulfonyl)propoxy)phenyl)-5-fluoro-2,3-dihydro-1H-inden-1-yl)oxy)-2,3-dihydrofuro[3,2-c]pyridin-3-yl)acetic acid

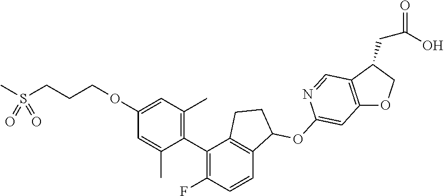 [SMILES: Cc1cc(OCCCS(C)(=O)=O)cc(C)c1-c1c(F)ccc2c1CCC2Oc1cc2c(cn1)[C@H](CC(=O)O)CO2]

Similar procedure to Example 3 was followed to arrive at the title compound, with the LC-MS[M+H]-m/z as 570.

### Example 61

### 2-((3S)-6-((4-(2,6-dimethyl-4-(3-(methylsulfonyl)propoxy)phenyl)-5-methoxy-2,3-dihydro-1H-inden-1-yl)oxy)-2,3-dihydrofuro[3,2-c]pyridin-3-yl)acetic acid

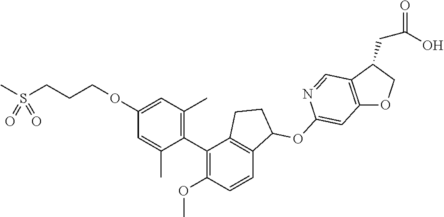 [SMILES: COc1ccc2c(c1-c1c(C)cc(OCCCS(C)(=O)=O)cc1C)CCC2Oc1cc2c(cn1)[C@H](CC(=O)O)CO2]

Similar procedure to Example 3 was followed to arrive at the title compound, with the LC-MS[M+H]-m/z as 582.

### Example 62

### 2-((3S)-6-((4-(2,6-dimethyl-4-(3-(methylsulfonyl)propoxy)phenyl)-7-(trifluoromethyl)-2,3-dihydro-1H-inden-1-yl)oxy)-2,3-dihydrofuro[3,2-c]pyridin-3-yl)acetic acid

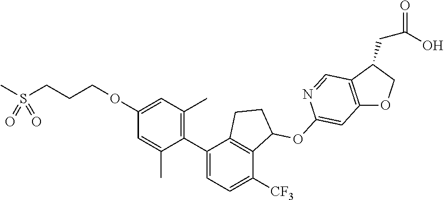 [SMILES: Cc1cc(OCCCS(C)(=O)=O)cc(C)c1-c1ccc(C(F)(F)F)c2c1CCC2Oc1cc2c(cn1)[C@H](CC(=O)O)CO2]

Similar procedure to Example 3 was followed to arrive at the title compound, with the LC-MS[M+H]-m/z as 620.

### Example 63

### 2-((3S)-6-((4-(3,5-dichloro-2,6-dimethyl-4-(3-(methylsulfonyl)propoxy)phenyl)-2,3-dihydro-1H-inden-1-yl)oxy)-2,3-dihydrofuro[3,2-c]pyridin-3-yl)acetic acid

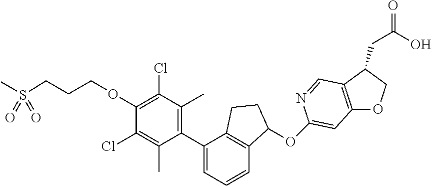 [SMILES: Cc1c(Cl)c(OCCCS(C)(=O)=O)c(Cl)c(C)c1-c1cccc2c1CCC2Oc1cc2c(cn1)[C@H](CC(=O)O)CO2]

Similar procedure to Example 3 was followed to arrive at the title compound, with the LC-MS[M+H]-m/z as 621.

### Example 64

### 2-((3R)-6-((4-(3-fluoro-2,6-dimethyl-4-(3-(methylsulfonyl)propoxy)phenyl)-2,3-dihydro-1H-inden-1-yl)oxy)-2,3-dihydrofuro[3,2-c]pyridin-3-yl)acetic acid

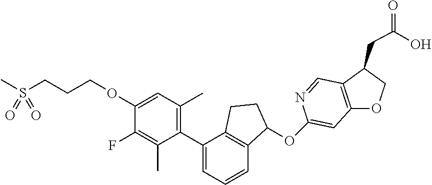 [SMILES: Cc1cc(OCCCS(C)(=O)=O)c(F)c(C)c1-c1cccc2c1CCC2Oc1cc2c(cn1)[C@@H](CC(=O)O)CO2]

Similar procedure to Example 3 was followed to arrive at the title compound, with the LC-MS[M+H]-m/z as 570.

### Example 65

### 2-((3R)-6-((4-(2-methyl-4-(3-(methylsulfonyl)porpoxy)phenyl)-2,3-dihydro-1H-inden-1-yl)oxy)-2,3-dihydrofuro[3,2-c]pyridin-3-yl)acetic acid

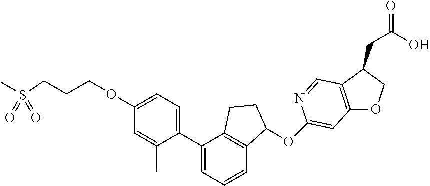 [SMILES: Cc1cc(OCCCS(C)(=O)=O)ccc1-c1cccc2c1CCC2Oc1cc2c(cn1)[C@@H](CC(=O)O)CO2]

Similar procedure to Example 3 was followed to arrive at the title compound, with the LC-MS[M+H]-m/z as 538.

### Example 66

### 2-((3S)-6-((4-(4-cyano-2,6-dimethylphenyl)-5,7-dimethyl-2,3-dihydro-1H-inden-1-yl)oxy)-2,3-dihydrofuro[3,2-c]pyridin-3-yl)acetic acid

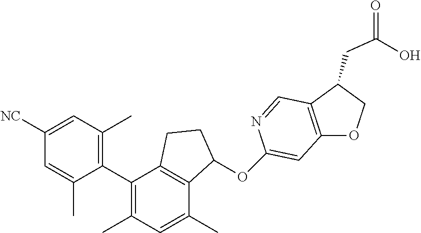 [SMILES: Cc1cc(C#N)cc(C)c1-c1c(C)cc(C)c2c1CCC2Oc1cc2c(cn1)[C@H](CC(=O)O)CO2]

Similar procedure to Example 3 was followed to arrive at the title compound, with the LC-MS[M+H]-m/z as 469.

### Example 67

### 2-((3S)-6-((4-(2,6-dimethyl-4-nitrophenyl)-2,3-dihydro-1H-inden-1-yl)oxy)-2,3-dihydrofuro[3,2-c]pyridin-3-yl)acetic acid

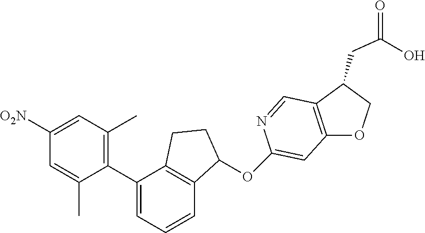 [SMILES: Cc1cc([N+](=O)[O-])cc(C)c1-c1cccc2c1CCC2Oc1cc2c(cn1)[C@H](CC(=O)O)CO2]

Similar procedure to Example 3 was followed to arrive at the title compound, with the LC-MS[M+H]-m/z as 461.

### Example 68

### 2-((3R)-6-((4-(4-bromo-2,6-dimethylphenyl)-2,3-dihydro-1H-inden-1-yl)oxy)-2,3-dihydrofuro[3,2-c]pyridin-3-yl)acetic acid

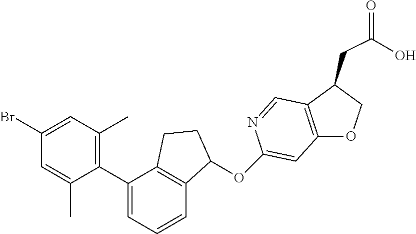 [SMILES: Cc1cc(Br)cc(C)c1-c1cccc2c1CCC2Oc1cc2c(cn1)[C@@H](CC(=O)O)CO2]

Similar procedure to Example 3 was followed to arrive at the title compound, with the LC-MS[M+H]-m/z as 495.

### Example 69

### 2-((3S)-6-((4-(2,3,5,6-tetramethyl-4-(3-(methylsulfonyl)propoxy)phenyl)-2,3-dihydro-1H-inden-1-yl)oxy)-2,3-dihydrofuro[3,2-c]pyridin-3-yl)acetic acid

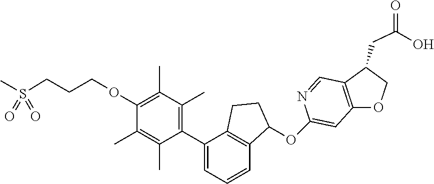 [SMILES: Cc1c(C)c(-c2cccc3c2CCC3Oc2cc3c(cn2)[C@H](CC(=O)O)CO3)c(C)c(C)c1OCCCS(C)(=O)=O]

Similar procedure to Example 3 was followed to arrive at the title compound, with the LC-MS[M+H]-m/z as 580.

### Example 70

### 2-((3S)-6-((4-(2-(hydroxymethyl)-6-methyl-4-(3-(methylsulfonyl)propoxy)phenyl)-2,3-dihydro-1H-inden-1-yl)oxy)-2,3-dihydrofuro[3,2-c]pyridin-3-yl)acetic acid

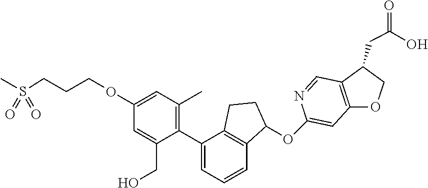 [SMILES: Cc1cc(OCCCS(C)(=O)=O)cc(CO)c1-c1cccc2c1CCC2Oc1cc2c(cn1)[C@H](CC(=O)O)CO2]

Similar procedure to Example 3 was followed to arrive at the title compound, with the LC-MS[M+H]-m/z as 568.

### Example 71

### 2-((3S)-6-((4-(2-methoxy-6-methyl-4-(3-(methylsulfonyl)propoxy)phenyl)-2,3-dihydro-1H-inden-1-yl)oxy)-2,3-dihydrofuro[3,2-c]pyridin-3-yl)acetic acid

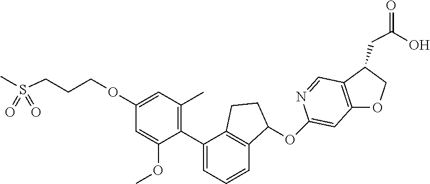 [SMILES: COc1cc(OCCCS(C)(=O)=O)cc(C)c1-c1cccc2c1CCC2Oc1cc2c(cn1)[C@H](CC(=O)O)CO2]

Similar procedure to Example 3 was followed to arrive at the title compound, with the LC-MS[M+H]-m/z as 568.

### Example 72

### 2-((3S)-6-((4-(4-(3-(methylsulfonyl)propoxy)-2,6-bis(trifluoromethyl)phenyl)-2,3-dihydro-1H-inden-1-yl)oxy)-2,3-dihydrofuro[3,2-c]pyridin-3-yl)acetic acid

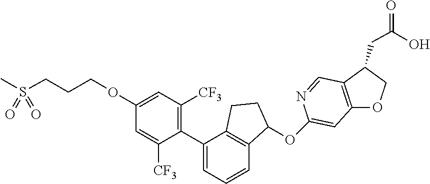 [SMILES: CS(=O)(=O)CCCOc1cc(C(F)(F)F)c(-c2cccc3c2CCC3Oc2cc3c(cn2)[C@H](CC(=O)O)CO3)c(C(F)(F)F)c1]

Similar procedure to Example 3 was followed to arrive at the title compound, with the LC-MS[M+H]-m/z as 660.

### Example 73

### 2-((3R)-6-((4-(2,6-dimethyl-4-(3-(methylsulfonyl)propoxy)phenyl)-2,3-dihydro-1H-inden-1-yl)oxy)-7-(trifluoromethyl)-2,3-dihydrofuro[3,2-c]pyridin-3-yl)acetic acid

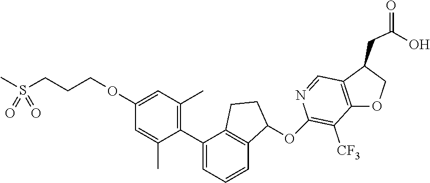 [SMILES: Cc1cc(OCCCS(C)(=O)=O)cc(C)c1-c1cccc2c1CCC2Oc1ncc2c(c1C(F)(F)F)OC[C@@H]2CC(=O)O]

Similar procedure to Example 3 was followed to arrive at the title compound, with the LC-MS[M+H]-m/z as 620.

### Example 74

### 2-((3R)-6-((4-(4-(3-(ethylsulfonyl)propoxy)-2,6-dimethylphenyl)-2,3-dihydro-1H-inden-1-yl)oxy)-2,3-dihydrofuro[3,2-c]pyridin-3-yl)acetic acid

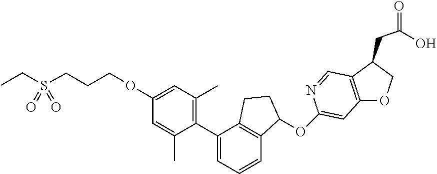 [SMILES: CCS(=O)(=O)CCCOc1cc(C)c(-c2cccc3c2CCC3Oc2cc3c(cn2)[C@@H](CC(=O)O)CO3)c(C)c1]

Similar procedure to Example 3 was followed to arrive at the title compound, with the LC-MS[M+H]-m/z as 566.

### Example 75

### 2-((3R)-6-((4-(2,6-diethyl-4-(3-(methylsulfonyl)propoxy)phenyl)-2,3-dihydro-1H-inden-1-yl)oxy)-2,3-dihydrofuro[3,2-c]pyridin-3-yl)acetic acid

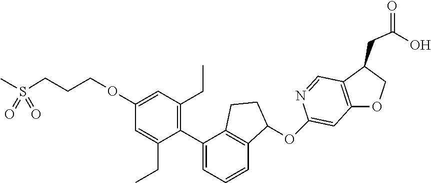 [SMILES: CCc1cc(OCCCS(C)(=O)=O)cc(CC)c1-c1cccc2c1CCC2Oc1cc2c(cn1)[C@@H](CC(=O)O)CO2]

Similar procedure to Example 3 was followed to arrive at the title compound, with the LC-MS[M+H]-m/z as 580.

### Example 76

### 2-((3R)-6-((4-(4-(3-(methylsulfonyl)propoxy)phenyl)-2,3-dihydro-1H-inden-1-yl)oxy)-2,3-dihydrofuro[3,2-c]pyridin-3-yl)acetic acid

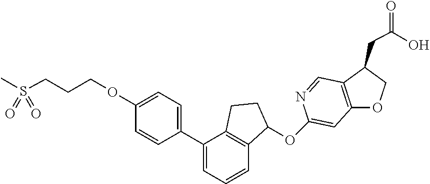 [SMILES: CS(=O)(=O)CCCOc1ccc(-c2cccc3c2CCC3Oc2cc3c(cn2)[C@@H](CC(=O)O)CO3)cc1]

Similar procedure to Example 3 was followed to arrive at the title compound, with the LC-MS[M+H]-m/z as 524.

### Example 77

### (S)-2-(7-methyl-6-((2′,4′,6′-trimethyl-[1,1′-biphenyl]-3-yl)methoxy)-2,3-dihydrofuro[3,2-c]pyridin-3-yl)acetic acid

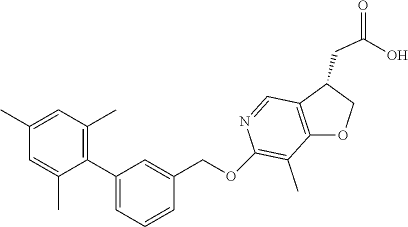 [SMILES: Cc1cc(C)c(-c2cccc(COc3ncc4c(c3C)OC[C@H]4CC(=O)O)c2)c(C)c1]

Similar procedure to Example 2 was followed to arrive at the title compound, with the LC-MS[M+H]-m/z as 418.

### Example 78

### (S)-2-(6-((4′-methoxy-2′,6′-dimethyl-[1,1′-biphenyl]-3-yl)methoxy)-2,3-dihydrofuro[3,2-c]pyridin-3-yl)acetic acid

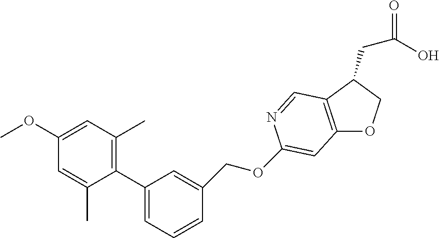 [SMILES: COc1cc(C)c(-c2cccc(COc3cc4c(cn3)[C@H](CC(=O)O)CO4)c2)c(C)c1]

Similar procedure to Example 2 was followed to arrive at the title compound, with the LC-MS[M+H]-m/z as 420.

### Example 79

### (S)-2-(7-bromo-6-((2′,6′-dimethyl-4′-(trifluoromethyl)-[1,1′-biphenyl]-3-yl)methoxy)-2,3-dihydrofuro[3,2-c]pyridin-3-yl)acetic acid

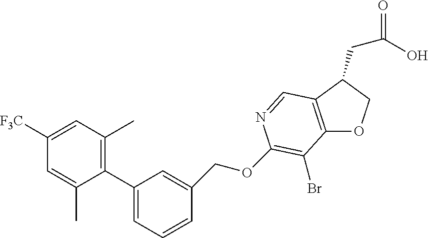 [SMILES: Cc1cc(C(F)(F)F)cc(C)c1-c1cccc(COc2ncc3c(c2Br)OC[C@H]3CC(=O)O)c1]

Similar procedure to Example 2 was followed to arri ve at the title compound, with the LC-MS[M+H]-m/z as 537.

### Example 80

### (S)-2-(6-((2′,6′-dimethyl-4′-(trifluoromethoxy)-[1,1′-biphenyl]-3-yl)methoxy)-2,3-dihydrofuro[3,2-c]pyridin-3-yl)acetic acid

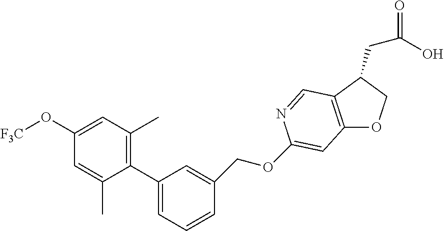 [SMILES: Cc1cc(OC(F)(F)F)cc(C)c1-c1cccc(COc2cc3c(cn2)[C@H](CC(=O)O)CO3)c1]

Similar procedure to Example 2 was followed to arrive at the title compound, with the LC-MS[M+H]-m/z as 474.

### Example 81

### (S)-2-(6-((4′-cyclohexyl-2′,5,6′-trimethyl-[1,1′-biphenyl]-3-yl)methoxy)-2,3-dihydrofuro[3,2-c]pyridin-3-yl)acetic acid

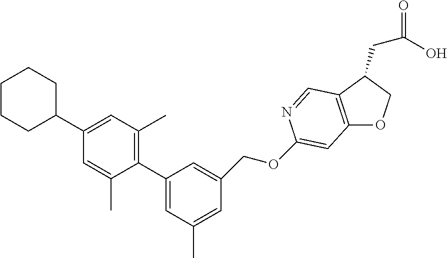 [SMILES: Cc1cc(COc2cc3c(cn2)[C@H](CC(=O)O)CO3)cc(-c2c(C)cc(C3CCCCC3)cc2C)c1]

Similar procedure to Example 2 was followed to arrive at the title compound, with the LC-MS[M+H]-m/z as 486.

### Example 82

### (S)-b 2-l -(6-((6-fluoro-2′,6′-dimethyl-4′-(3-(methylsulfonyl)propoxy)-[1,1′-biphenyl]-3-yl)methoxy)-2,3-dihydrofuro[3,2-c]pyridin-3-yl)acetic acid

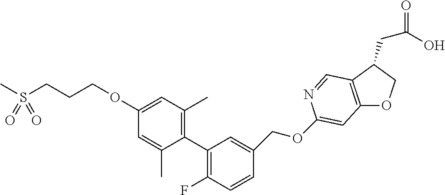 [SMILES: Cc1cc(OCCCS(C)(=O)=O)cc(C)c1-c1cc(COc2cc3c(cn2)[C@H](CC(=O)O)CO3)ccc1F]

Similar procedure to Example 2 was followed to arrive at the title compound, with the LC-MS[M+H]-m/z as 544.

### Example 83

### (S)-2-(6-((6-methoxy-2′,6′-dimethyl-4′-(3-(methylsulfonyl)propoxy)-[1,1′-biphenyl]-3-yl)methoxy)-2,3-dihydrofuro[3,2-c]pyridin-3-yl)acetic acid

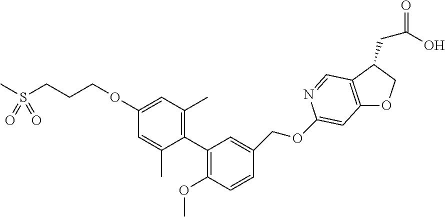 [SMILES: COc1ccc(COc2cc3c(cn2)[C@H](CC(=O)O)CO3)cc1-c1c(C)cc(OCCCS(C)(=O)=O)cc1C]

Similar procedure to Example 2 was followed to arrive at the title compound, with the LC-MS[M+H]-m/z as 556.

### Example 84

### (S)-2-(6-((2′,6′-dimethyl-4′-(3-(methylsulfonyl)porpoxy)phenyl)4-(trifluoromethyl)-[1,1′-biphenyl]-3-yl)methoxy)-2,3-dihydrofuro[3,2-c]pyridin-3-yl)acetic acid

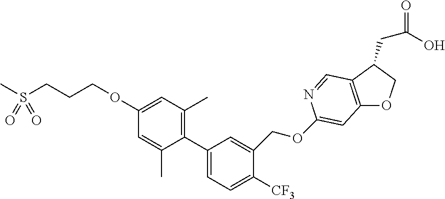 [SMILES: Cc1cc(OCCCS(C)(=O)=O)cc(C)c1-c1ccc(C(F)(F)F)c(COc2cc3c(cn2)[C@H](CC(=O)O)CO3)c1]

Similar procedure to Example 2 was followed to arrive at the title compound, with the LG-MS[M+H]-m/z as 594.

### Example 85

### (S)-2-(6-((3′,5′-dichloro-2′,6′-dimethyl-4′-(3-(methylsulfonyl)propoxy)-[1,1′-biphenyl]-3-yl)methoxy)-2,3-dihydrofuro[3,2-c]pyridin-3-yl)acetic acid

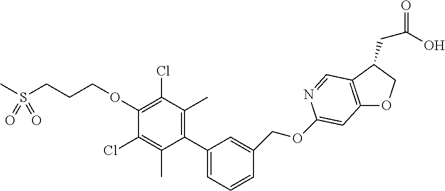 [SMILES: Cc1c(Cl)c(OCCCS(C)(=O)=O)c(Cl)c(C)c1-c1cccc(COc2cc3c(cn2)[C@H](CC(=O)O)CO3)c1]

Similar procedure to Example 2 was followed to arrive at the title compound, with the LC-MS[M+H]-m/z as 595.

### Example 86

### (S)-2-(6-((3′-fluoro-2′,6′-dimethyl-4′-(3-(methylsulfonyl)propoxy)-[1,1′-biphenyl]-3-yl)methoxy)-2,3-dihydrofuro[3,2-c]pyridin-3-yl)acetic acid

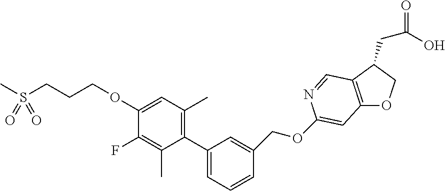 [SMILES: Cc1cc(OCCCS(C)(=O)=O)c(F)c(C)c1-c1cccc(COc2cc3c(cn2)[C@H](CC(=O)O)CO3)c1]

Similar procedure to Example 2 was followed to arri ve at the title compound, with the LC-MS[M+H]-m/z as 544.

### Example 87

### (S)-2-(6-((2′-methyl-4′-(3-(methylsulfonyl)propoxy)-[1,1′-biphenyl]-3-yl)methoxy)-2,3-dihydrofuro[3,2-c]pyridin-3-yl)acetic acid

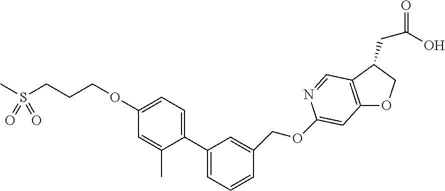 [SMILES: Cc1cc(OCCCS(C)(=O)=O)ccc1-c1cccc(COc2cc3c(cn2)[C@H](CC(=O)O)CO3)c1]

Similar procedure to Example 2 was followed to arrive at the title compound, with the LC-MS[M+H]-m/z as 512.

### Example 88

### (S)-2-(6-((4′-cyano-2′,4,6,6′-tetramethyl-[1,1′-biphenyl]-3-yl)methoxy)-2,3-dihydrofuro[3,2-c]pyridin-3-yl)acetic acid

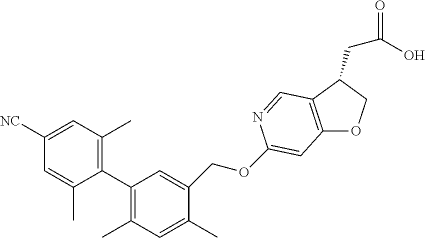 [SMILES: Cc1cc(C)c(-c2c(C)cc(C#N)cc2C)cc1COc1cc2c(cn1)[C@H](CC(=O)O)CO2]

Similar procedure to Example 2 was followed to arrive at the title compound, with the LC-MS[M+H]-m/z as 443.

### Example 89

### (S)-2-(6-((2′,6′-dimethyl-4′-nitro-[1,1′-biphenyl]-3-yl)methoxy)-2,3-

dihydrofuro[3,2-c]pyridin-3-yl)acetic acid

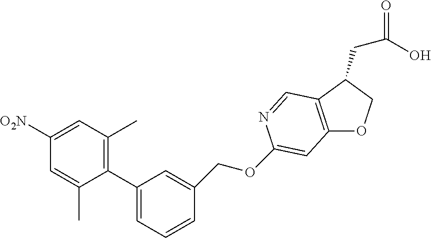 [SMILES: Cc1cc([N+](=O)[O-])cc(C)c1-c1cccc(COc2cc3c(cn2)[C@H](CC(=O)O)CO3)c1]

Similar procedure to Example 2 was followed to arrive at the title compound, with the LC-MS[M+H]-m/z as 434.

### Example 90

### (S)-2-(6-((4′-bromo-2′,6′-dimethyl-[1,1′-biphenyl]-3-yl)methoxy)-2,3-dihydrofuro[3,2-c]pyridin-3-yl)acetic acid

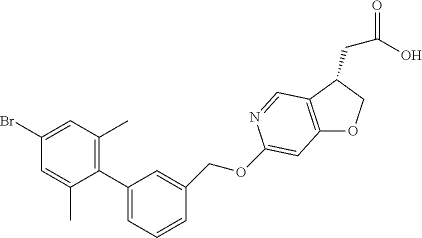 [SMILES: Cc1cc(Br)cc(C)c1-c1cccc(COc2cc3c(cn2)[C@H](CC(=O)O)CO3)c1]

Similar procedure to Example 2 was followed to arrive at the title compound, with the LC-MS[M+H]-m/z as 469.

### Example 91

### (S)-2-(6-((2′,3′,5′,6′-tetramethyl-4′-(3-(methylsulfonyl)propoxy)-[1,1′-biphenyl]-3-yl)methoxy)-2,3-dihydrofuro[3,2-c]pyridin-3-yl)acetic acid

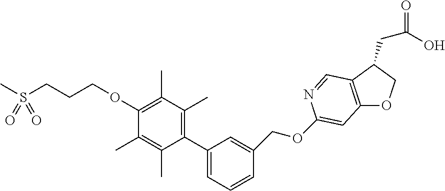 [SMILES: Cc1c(C)c(-c2cccc(COc3cc4c(cn3)[C@H](CC(=O)O)CO4)c2)c(C)c(C)c1OCCCS(C)(=O)=O]

Similar procedu re to Example 3 was followed to arrive at the title compound, with the LC-MS[M+H]-m/z as 554.

### Example 92

### (S)-2-(6-((2′-(hydroxymethyl)-6′-methyl-4′-(3-(methylsulfonyl)propoxy)-[1,1′-biphenyl]-3-yl)methoxy)-2,3-dihyddrofuro[3,2-c]pyridin-3-yl)acetic acid

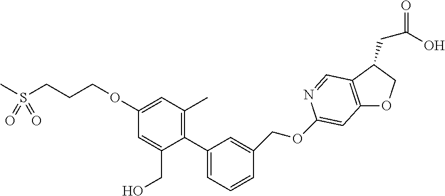 [SMILES: Cc1cc(OCCCS(C)(=O)=O)cc(CO)c1-c1cccc(COc2cc3c(cn2)[C@H](CC(=O)O)CO3)c1]

Similar procedure to Example 2 was followed to arrive at the title compound, with the LC-MS[M+H]m/z as 542.

### Example 93

### (S)-2-(6-((2′-methoxy-6′-methyl-4′-(3-(methylsulfonyl)propoxy)-[1,1′-biphenyl]-3-yl)methoxy)-2,3-dihydrofuro[3,2-c]pyridin-3-yl)acetic acid

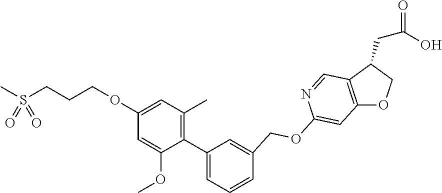 [SMILES: COc1cc(OCCCS(C)(=O)=O)cc(C)c1-c1cccc(COc2cc3c(cn2)[C@H](CC(=O)O)CO3)c1]

Similar procedure to Example 2 was followed to arrive at the title compound, with the LC-MS[M+H]-m/z as 542.

### Example 94

### (S)-2-(6-((4′-(3-(methylsulfonyl)propoxy)-2′,6′-bis(trifluoromethyl)-[1,1′-biphenyl]-3-yl)methoxy)-2,3-dihydrofuro[3,2-c]pyridin-3-yl)acetic acid

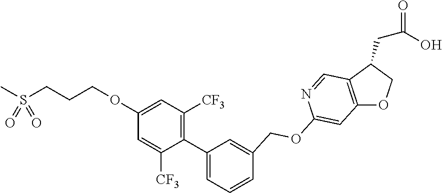 [SMILES: CS(=O)(=O)CCCOc1cc(C(F)(F)F)c(-c2cccc(COc3cc4c(cn3)[C@H](CC(=O)O)CO4)c2)c(C(F)(F)F)c1]

Similar procedure to Example 2 was followed to arrive at the title compound, with the LC-MS[M+H]-m/z as 634.

### Example 95

### (S)-2-(6-((2′,6′-dimethyl-4′-(3-(methylsulfonyl)propoxy)-[1,1′-biphenyl]-3-yl)methoxy)-7-(trifluoromethyl)-2,3-dihydrofuro[3,2-c]pyridin-3-yl)acetic acid

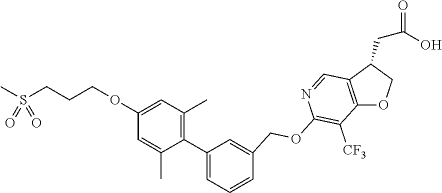 [SMILES: Cc1cc(OCCCS(C)(=O)=O)cc(C)c1-c1cccc(COc2ncc3c(c2C(F)(F)F)OC[C@H]3CC(=O)O)c1]

Similar procedure to Example 2 was followed to arrive at the title compound, with the LC-MS[M+H]-m/z as 594.

### Example 96

### (S)-2-(6-((4′-(3-(ethylsulfonyl)propoxy)-2′,6′-dimethyl-[1,1′-biphenyl]-3-yl)methoxy)-2,3-dihydrofuro[3,2-c]pyridin-3-yl)acetic acid

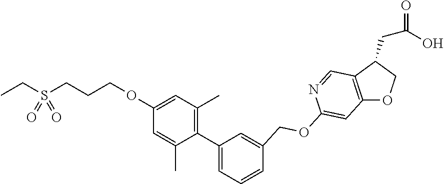 [SMILES: CCS(=O)(=O)CCCOc1cc(C)c(-c2cccc(COc3cc4c(cn3)[C@H](CC(=O)O)CO4)c2)c(C)c1]

Similar procedure to Example 2 was followed to arrive at the title compound, with the LC-MS[M+H]-m/z as 540.

### Example 97

### (S)-2-(6-((2′,6′-diethyl-4′-(3-(methylsulfonyl)propoxy)-[1,1′-biphenyl]-3-yl)methoxy)-2,3-dihydrofuro[3,2-c]pyridin-3-yl)acetic acid

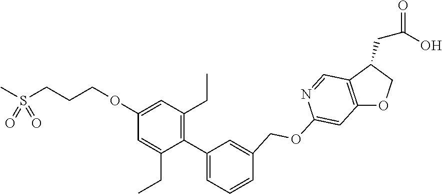 [SMILES: CCc1cc(OCCCS(C)(=O)=O)cc(CC)c1-c1cccc(COc2cc3c(cn2)[C@H](CC(=O)O)CO3)c1]

Similar procedure to Example 2 was followed to arrive at the title compound, with the LC-MS[M+H]-m/z as 554.

### Example 98

### (S)-2-(6-((4′-(3-(methylsulfonyl)propoxy)-[1,1′-biphenyl]-3-yl)methoxy)-2,3-dihydrofuro[3,2-c]pyridin-3-yl)acetic acid

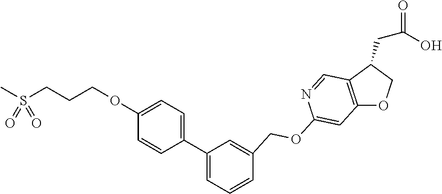 [SMILES: CS(=O)(=O)CCCOc1ccc(-c2cccc(COc3cc4c(cn3)[C@H](CC(=O)O)CO4)c2)cc1]

Similar procedure to Example 2 was followed to arrive at the title compound, with the LC-MS[M+H]-m/z as 498.

### Example 99

### 2-((3S)-6-((4-mesityl-2,3-dihydro-1H-inden-1-yl)oxy)-7-methyl-2,3-dihydrothieno[3,2-c]pyridin-3-yl)acetic acid

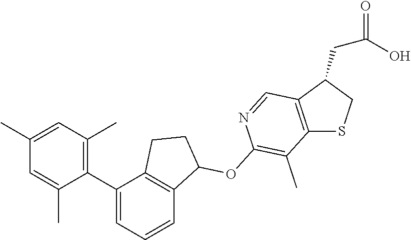 [SMILES: Cc1cc(C)c(-c2cccc3c2CCC3Oc2ncc3c(c2C)SC[C@H]3CC(=O)O)c(C)c1]

Similar procedu re to Example 3 was followed to arrive at the title compound, with the LC-MS[M+H]-m/z as 460.

### Example 100

### 2-((3S)-6-((4-(4-methoxy-2,6-dimethylphenyl)-2,3-dihydro-1H-inden-1-yl)oxy)-2,3-dihydrothieno[3,2-c]pyridin-3-yl)acetic acid

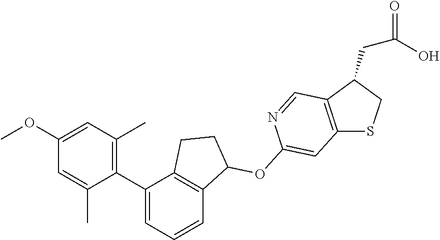 [SMILES: COc1cc(C)c(-c2cccc3c2CCC3Oc2cc3c(cn2)[C@H](CC(=O)O)CS3)c(C)c1]

Similar procedure to Example 3 was followed to arrive at the title compound, with the LC-MS[M+H]-m/z as 462.

### Example 101

### 2-((3S)-7-bromo-6-((4-(2,6-dimethyl-4-(trifluoromethyl)phenyl)-2,3-dihydro-1H-inden-1-yl)oxy)-2,3-dihydrothieno[3,2-c]pyrldin-3-yl)acetic acid

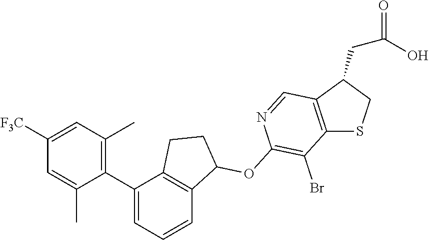 [SMILES: Cc1cc(C(F)(F)F)cc(C)c1-c1cccc2c1CCC2Oc1ncc2c(c1Br)SC[C@H]2CC(=O)O]

Similar procedure to Example 3 was followed to arrive at the title compound, with the LC-MS[M+H]-m/z as 579.

### Example 102

### 2-((3S)-6-((4-(2,6-dimethyS-4-(trifluoromethoxy)phenyl)-2,3-dihydro-1H-inden-1-yl)oxy)-2,3-dihydrothieno[3,2-c]pyridin-3-yl)acetic acid

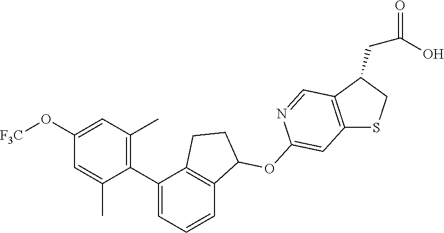 [SMILES: Cc1cc(OC(F)(F)F)cc(C)c1-c1cccc2c1CCC2Oc1cc2c(cn1)[C@H](CC(=O)O)CS2]

Similar procedure to Example 3 was followed to arrive at the title compound, with the LC-MS[M+H]-m/z as 516.

### Example 103

### 2-((3S)-6-((4-(4-cyclohexyl-2,6-dimethylphenyl)-6-methyl-2,3-dihydro-1H-inden-1-yl)oxy)-2,3-dihydrothieno[3,2-c]pyridin-3-yl)acetic acid

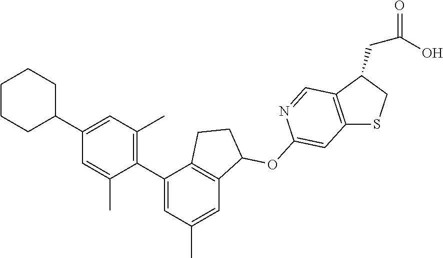 [SMILES: Cc1cc(-c2c(C)cc(C3CCCCC3)cc2C)c2c(c1)C(Oc1cc3c(cn1)[C@H](CC(=O)O)CS3)CC2]

Similar procedure to Example 3 was followed to arrive at the title compound, with the LC-MS[M+H]-m/z as 528.

### Example 104

### 2-((3S)-6-((4-(2,6-dimethyl-4-(3-(methylsulfonyl)propoxy)phenyl)-5-fluoro-2,3-dihydro-1H-inden-1-yl)oxy)-2,3-dihydrothieno[3,2-c]pyridin-3-yl)acetic acid

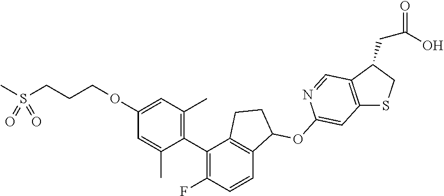 [SMILES: Cc1cc(OCCCS(C)(=O)=O)cc(C)c1-c1c(F)ccc2c1CCC2Oc1cc2c(cn1)[C@H](CC(=O)O)CS2]

Similar procedure to Example 3 was followed to arrive at the title compound, with the LC-MS[M+H]-m/z as 586.

### Example 105

### 2-((3S)-6-((4-(2,6-dimethyl-4-(3-(methylsulfonyl)propoxy)phenyl)-5-methoxy-2,3-dihydro-1H-inden-1-yl)oxy)-2,3-difydrothieno[3,2-c]pyridin-3-yl)acetic acid

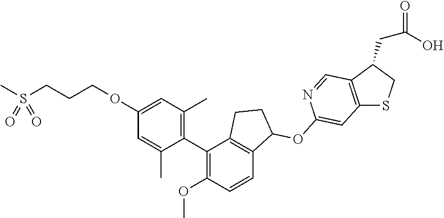 [SMILES: COc1ccc2c(c1-c1c(C)cc(OCCCS(C)(=O)=O)cc1C)CCC2Oc1cc2c(cn1)[C@H](CC(=O)O)CS2]

Similar procedure to Example 3 was followed to arrive at the title compound, with the LC-MS[M+H]-m/z as 598.

### Example 106

### 2-((3S)-6-((4-(2,6-dimethyl-4-(3-(methylsulfonyl)propoxy)phenyl)-7-(trifluoromethyl)-2,3-dihydro-1H-inden-1-yl)oxy)-2,3-dihydrothieno[3,2-c]pyridin-3-yl)acetic acid

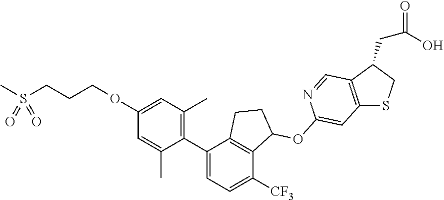 [SMILES: Cc1cc(OCCCS(C)(=O)=O)cc(C)c1-c1ccc(C(F)(F)F)c2c1CCC2Oc1cc2c(cn1)[C@H](CC(=O)O)CS2]

Similar procedure to Example 3 was followed to arrive at the title compound, with the LC-MS[M+H]-m/z as 636.

### Example 107

### 2-((3S)-6-((4-(3,5-dichloro-2,6-dimethyl-4-(3-(methylsulfonyl)propoxy)phenyl)-2,3-dihydro-1H-inden-1-yl)oxy)-2,3-dihydrothieno[3,2-c]pyridin-3-yl)acetic acid

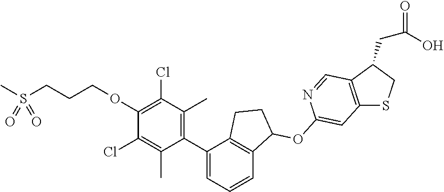 [SMILES: Cc1c(Cl)c(OCCCS(C)(=O)=O)c(Cl)c(C)c1-c1cccc2c1CCC2Oc1cc2c(cn1)[C@H](CC(=O)O)CS2]

Similar procedure to Example 3 was followed to arrive at the title compound, with the LC-MS[M+H]-m/z as 637.

### Example 108

### 2-((3S)-6-((4-(3-fluoro-2,6-dlmettiyl-4-(3-(methylsulfonyl)propoxy)phenyl)-2,3-dihydro-1H-inden-1-yl)oxy)-2,3-dihydrothieno[3,2-c]pyridin-3-yl)acetic acid

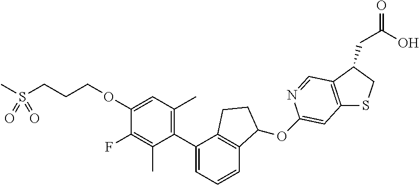 [SMILES: Cc1cc(OCCCS(C)(=O)=O)c(F)c(C)c1-c1cccc2c1CCC2Oc1cc2c(cn1)[C@H](CC(=O)O)CS2]

Similar procedure to Example 3 was followed to arrive at the title compound, with the LC-MS[M+H]-m/z as 586.

### Example 109

### 2-((3S)-6-((4-(2-methyl-4-(3-(methylsulfonyl)propoxy)phenyl)-2,3-dihydro-1H-inden-1-yl)oxy)-2,3-dihydrothieno[3,2-c]pyridin-3-yl)acetic acid

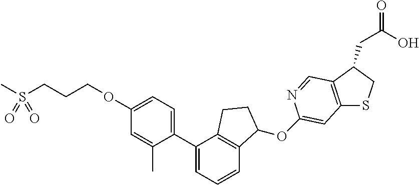 [SMILES: Cc1cc(OCCCS(C)(=O)=O)ccc1-c1cccc2c1CCC2Oc1cc2c(cn1)[C@H](CC(=O)O)CS2]

Similar procedure to Example 3 was followed to arrive at the title compound, with the LC-MS[M+H]-m/z as 554.

### Example 110

### 2-((3S)-6-((4-(4-cyano-2,6-dimethylphenyl)-5,7-dimethyl-2,3-dihydro-1H-indden-1-yl)oxy)-2,3-dihydrothieno[3,2-c]pyridin-3-yl)acetic acid

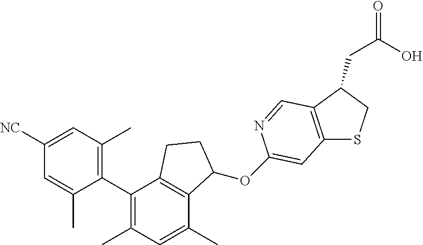 [SMILES: Cc1cc(C#N)cc(C)c1-c1c(C)cc(C)c2c1CCC2Oc1cc2c(cn1)[C@H](CC(=O)O)CS2]

Similar procedure to Example 3 was followed to arrive at the title compound, with the LC-MS[M+H]-m/z as 485.

### Example 111

### 2-((3S)-6-((4-(2,6-dimethyl-4-nitrophenyl)-2,3-dihydro-1H-inden-1-yl)oxy)-2,3-dihydrothieno[3,2-c]pyridin-3-yl)acetic acid

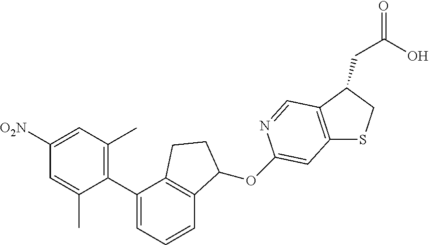 [SMILES: Cc1cc([N+](=O)[O-])cc(C)c1-c1cccc2c1CCC2Oc1cc2c(cn1)[C@H](CC(=O)O)CS2]

Similar procedure to Example 3 was followed to arrive at the title compound, with the LC-MS[M+H]-m/z as 477.

### Example 112

### 2-((3S)-6-((4-(4-bromo-2,6-dimethylphenyl)-2,3-dihydro-1H-inden-1-yl)oxy)-2,3-dihydrothieno[3,2-c]pyridin-3-yl)acetic acid

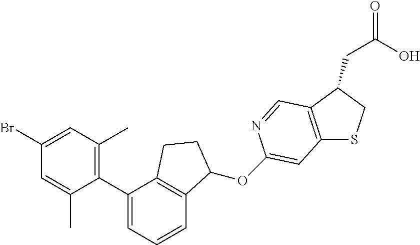 [SMILES: Cc1cc(Br)cc(C)c1-c1cccc2c1CCC2Oc1cc2c(cn1)[C@H](CC(=O)O)CS2]

Similar procedure to Example 3 was followed to arrive at the title compound, with the LC-MS[M+H]-m/z as 511.

### Example 113

### 2-((3S)-6-((4-(2,3,5,6-tetramethyl-4-(3-(methylsulfonyl)propoxy)phenyl)-2,3-dihydro-1H-inden-1-yl)oxy)-2,3-dihydrothieno[3,2-c]pyridin-3-yl)acetic acid

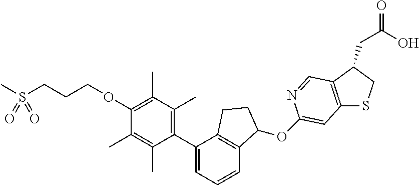 [SMILES: Cc1c(C)c(-c2cccc3c2CCC3Oc2cc3c(cn2)[C@H](CC(=O)O)CS3)c(C)c(C)c1OCCCS(C)(=O)=O]

Similar procedure to Example 3 was followed to arrive at the title compound, with the LC-MS[M+H]-m/z as 596.

### Example 114

### 2-((3S)-6-((4-(2-(hydroxymethyl)-6-methyl-4-(3-(methylsulfonyl)propoxy)phenyl)-2,3-dihydro-1H-inden-1-yl)oxy)-2,3-dihydrothieno[3,2-c]pyridin-3-yl)acetic acid

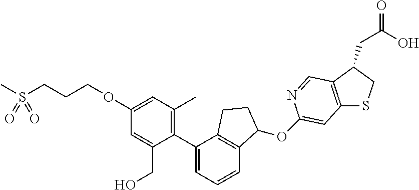 [SMILES: Cc1cc(OCCCS(C)(=O)=O)cc(CO)c1-c1cccc2c1CCC2Oc1cc2c(cn1)[C@H](CC(=O)O)CS2]

Similar procedure to Example 3 was followed to arrive at the title compound, with the LC-MS[M+H]-m/z as 584.

### Example 115

### 2-((3S)-6-((4-(2-methoxy-6-methyl-4-(3-(methylsulfonyl)propoxy)phenyl)-2,3-dihydro-1H-inden-1-yl)oxy)-2,3-dihydrothieno[3,2-c]pyridin-3-yl)acetic acid

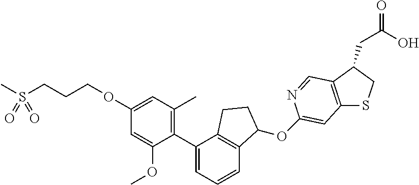 [SMILES: COc1cc(OCCCS(C)(=O)=O)cc(C)c1-c1cccc2c1CCC2Oc1cc2c(cn1)[C@H](CC(=O)O)CS2]

Similar procedure to Example 3 was followed to arrive at the title compound, with the LC-MS[M+H]-m/z as 584.

### Example 116

### 2-((3S)-6-((4-(4-(3-(methylsulfonyl)propoxy)-2,6-bis(trifluoromethyl)phenyl)-2,3-dihydro-1H-inden-1-yl)oxy)-2,3-dihydrothieno[3,2-c]pyridin-3-yl)acetic acid

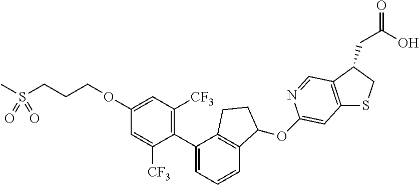 [SMILES: CS(=O)(=O)CCCOc1cc(C(F)(F)F)c(-c2cccc3c2CCC3Oc2cc3c(cn2)[C@H](CC(=O)O)CS3)c(C(F)(F)F)c1]

Similar procedure to Example 3 was followed to arrive at the title compound, with the LC-MS[M+H]-m/z as 676.

### Example 117

### 2-((3S)-6-((4-(2,6-dimethyl-4-(3-(methylsulfonyl)propoxy)phenyl)-2,3-dihydro-1H-inden-1-yl)oxy)-7-(trifluoromethyl)-2,3-dihydrothieno[3,2-c]pyridin-3-yl)acetic acid

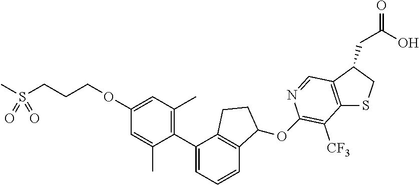 [SMILES: Cc1cc(OCCCS(C)(=O)=O)cc(C)c1-c1cccc2c1CCC2Oc1ncc2c(c1C(F)(F)F)SC[C@H]2CC(=O)O]

Similar procedure to Example 3 was followed to arrive at the title compound, with the LC-MS[M+H]-m/z as 636.

### Example 118

### 2-((3S)-6-((4-(4-(3-(ethylsulfonyl)porpoxy)-2,6-dimethylphenyl)-2,3-dihydro-1H-inden-1yl)oxy)2,3-dihydrothieno[3,2-c]pyridin-3-yl)acetic acid

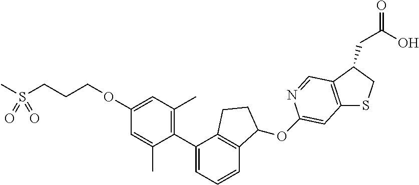 [SMILES: Cc1cc(OCCCS(C)(=O)=O)cc(C)c1-c1cccc2c1CCC2Oc1cc2c(cn1)[C@H](CC(=O)O)CS2]

Similar procedure to Example 3 was followed to arrive at the title compound, with the LC-MS[M+H]-m/z as 582.

### Example 119

### 2-((3S)-6-((4-(2,6-diethyl-4-(3-(methylsulfonyl)propoxy)phenyl)-2,3-dihydro-1H-inden-1-yl)oxy)-2,3-dihydrothieno[3,2-c]pyridin-3-yl)acetic acid

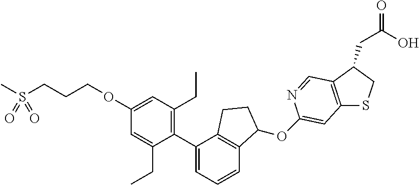 [SMILES: CCc1cc(OCCCS(C)(=O)=O)cc(CC)c1-c1cccc2c1CCC2Oc1cc2c(cn1)[C@H](CC(=O)O)CS2]

Similar procedure to Example 3 was followed to arrive at the title compound, with the LC-MS[M+H]-m/z as 596.

### Example 120

### 2-((3S)-6-((4-(4-(3-(methylsulfonyl)propoxy)phenyl)-2,3-dihydro-1H-inden-1-yl)oxy)-2,3-dihydrothieno[3,2-c]pyridin-3-yl)acetic acid

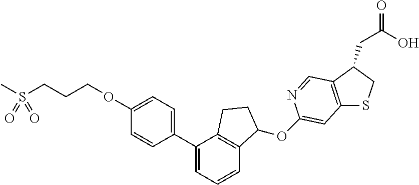 [SMILES: CS(=O)(=O)CCCOc1ccc(-c2cccc3c2CCC3Oc2cc3c(cn2)[C@H](CC(=O)O)CS3)cc1]

Similar procedu re to Example 3 was followed to arrive at the title compound, with the LC-MS[M+H]-m/z as 540.

### Example 121

### (S)-2-(7-methyl-6-((2′,4′,6′-trimethyl-[1,1′-biphenyl]-3-yl)methoxy)-2,3-dihydrothieno[3,2-c]pyridin-3-yl)acetic acid

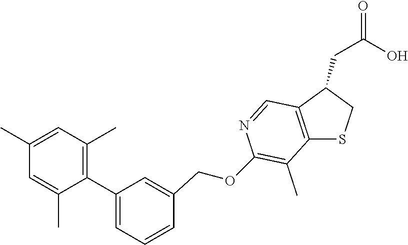 [SMILES: Cc1cc(C)c(-c2cccc(COc3ncc4c(c3C)SC[C@H]4CC(=O)O)c2)c(C)c1]

Similar procedure to Example 2 was followed to arrive at the title compound, with the LC-MS[M+H]-m/z as 434.

### Example 122

### (S)-2-(6-((4′-methoxy-2′,6′-dimethyl-[1,1′-biphenyl]-3-yl)methoxy)-2,3-dihydrothieno[3,2-c]pyridin-3-yl)acetic acid

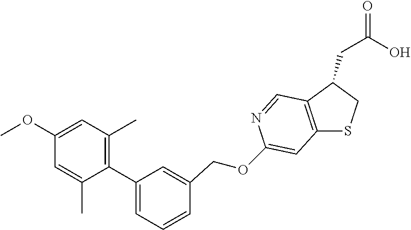 [SMILES: COc1cc(C)c(-c2cccc(COc3cc4c(cn3)[C@H](CC(=O)O)CS4)c2)c(C)c1]

Similar procedure to Example 2 was followed to arrive at the title compound, with the LC-MS[M+H]-m/z as 436.

### Example 123

### (S)-2-(7-bromo-6-((2′,6′-dimethyl-4′-(trifluoromethyl)-[1,1′-biphenyl]-3-yl)methoxy)-2,3-dihydrothieno[3,2-c]pyridin-3-yl)acetic acid

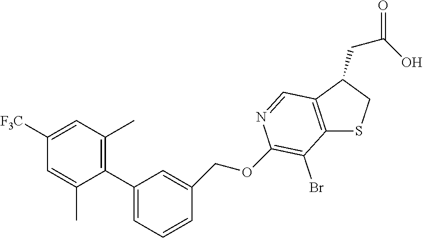 [SMILES: Cc1cc(C(F)(F)F)cc(C)c1-c1cccc(COc2ncc3c(c2Br)SC[C@H]3CC(=O)O)c1]

Similar procedure to Example 2 was followed to arrive at the title compound, with the LC-MS[M+H]-m/z as 553.

### Example 124

### (S)-2-(6-((2′,6′-dimethyl-4′-(trifluoromethoxy)-[1,1′-biphenyl]-3-yl)methoxy)-2,3-dihydrothieno[3,2-c]pyridin-3-yl)acetic acid

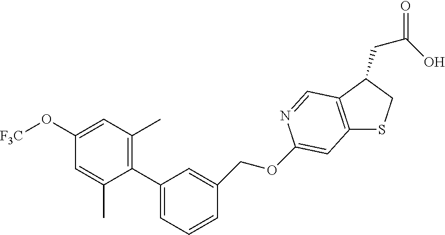 [SMILES: Cc1cc(OC(F)(F)F)cc(C)c1-c1cccc(COc2cc3c(cn2)[C@H](CC(=O)O)CS3)c1]

Similar procedure to Example 2 was followed to arrive at the title compound, with the LC-MS[M+H]-m/z as 490.

### Example 125

### (S)-2-(6-((4′-cyclohexyl-2′,5,6′-trimethyl-[1,1′-biphenyl]-3-yl)methoxy)-2,3-dihydrothieno[3,2-c]pyridin-3-yl)acetic acid

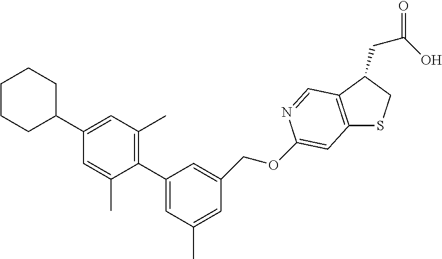 [SMILES: Cc1cc(COc2cc3c(cn2)[C@H](CC(=O)O)CS3)cc(-c2c(C)cc(C3CCCCC3)cc2C)c1]

Similar procedure to Example 2 was followed to arrive at the title compound, with the LC-MS[M+H]-m/z as 502.

### Example 126

### (S)-2-(6-((6-fluoro-2′,6′-dimethyl-4-(3-(methylsulfonyl)propoxy)-[1,1′-biphenyl]-3-yl)methoxy)-2,3-dihydrothieno[3,2-c]pyridin-3-yl)acetic acid

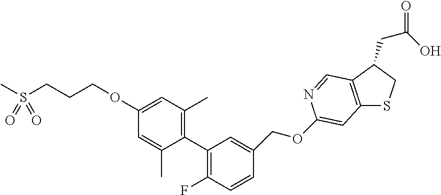 [SMILES: Cc1cc(OCCCS(C)(=O)=O)cc(C)c1-c1cc(COc2cc3c(cn2)[C@H](CC(=O)O)CS3)ccc1F]

Similar procedure to Example 2 was followed to arrive at the title compound, with the LC-MS[M+H]-m/z as 560.

### Example 127

### (S)-2-(6-((6-methoxy-2′,6′-dimethyl-4′-(3-(methylsulfonyl)propoxy)-[1,1′-biphenyl]-3-yl)methoxy)-2,3-dihydrothieno[3,2-c]pyridin-3-yl)acetic acid

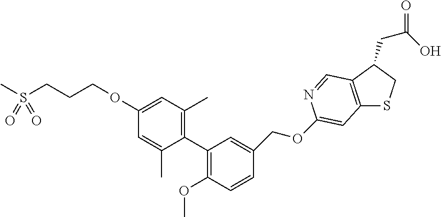 [SMILES: COc1ccc(COc2cc3c(cn2)[C@H](CC(=O)O)CS3)cc1-c1c(C)cc(OCCCS(C)(=O)=O)cc1C]

Similar procedure to Example 2 was followed to arrive at the title compound, with the LC-MS[M+H]-m/z as 572.

### Example 128

### (S)-2-(6-((2′,6′-dimethyl-4′-(3-(methylsulfonyl)propoxy)-4-(trifluoromethyl)-[1,1′-biphenyl]-3-yl)methoxy)-2,3-dihydrothieno[3,2-c]pyridin-3-yl)acetic acid

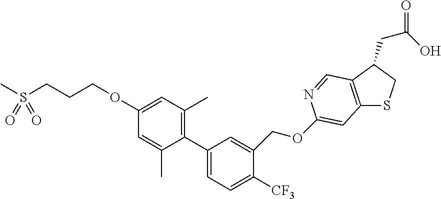 [SMILES: Cc1cc(OCCCS(C)(=O)=O)cc(C)c1-c1ccc(C(F)(F)F)c(COc2cc3c(cn2)[C@H](CC(=O)O)CS3)c1]

Similar procedure to Example 2 was followed to arrive at the title compound, with the LC-MS[M+H]-m/z as 610.

### Example 129

### (S)-2-(6-((3′,5′-dichloro-2′,6′-dimethyl-4′-(3-(methylsulfonyl)propoxy)-[1,1′-biphenyl]-3-yl)methoxy)-2,3-dihydrothieno[3,2-c]pyridin-3-yl)acetic acid

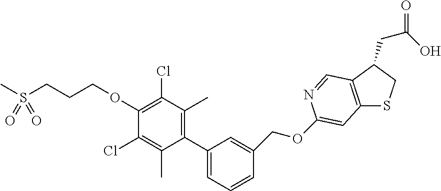 [SMILES: Cc1c(Cl)c(OCCCS(C)(=O)=O)c(Cl)c(C)c1-c1cccc(COc2cc3c(cn2)[C@H](CC(=O)O)CS3)c1]

Similar procedure to Example 2 was followed to arrive at the title compound, with the LC-MS[M+H]-m/z as 611.

### Example 130

### (S)-2-(6-((3′-fluoro-2′,6′-dimethyl-4′-(3-(methylsulfonyl)propoxy)-[1,1′-biphenyl]-3-yl)methoxy)-2,3-dihydrothieno[3,2-c]pyridin-3-yl)acetic acid

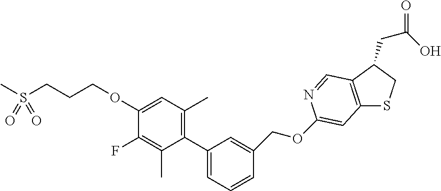 [SMILES: Cc1cc(OCCCS(C)(=O)=O)c(F)c(C)c1-c1cccc(COc2cc3c(cn2)[C@H](CC(=O)O)CS3)c1]

Similar procedure to Example 2 was followed to arrive at the title compound, with the LC-MS[M+H]-m/z as 560.

### Example 131

### (S)-2-(6-((2′-methyl-4′-(3-(methylsulfonyl)propoxy)-[1,1′-biphenyl]-3-yl)methoxy)-2,3-dihydrothieno[3,2-c]pyridin-3-yl)acetic acid

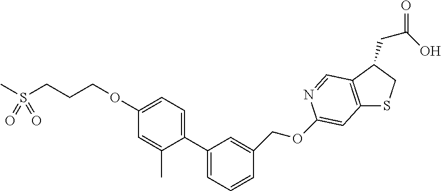 [SMILES: Cc1cc(OCCCS(C)(=O)=O)ccc1-c1cccc(COc2cc3c(cn2)[C@H](CC(=O)O)CS3)c1]

Similar procedure to Example 2 was followed to arrive at the title compound, with the LC-MS[M+H]-m/z as 528.

### Example 132

### (S)-2-(6-((4′-cyano-2′,4,6,6′-tetramethyl-[1,1′-biphenyl]-3-yl)methoxy)-2,3-dihydrothieno[3,2-c]pyridin-3-yl)acetic acid

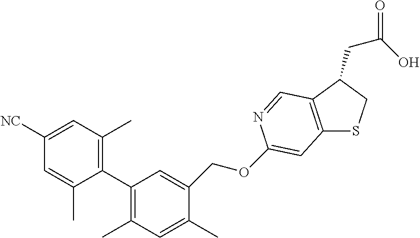 [SMILES: Cc1cc(C)c(-c2c(C)cc(C#N)cc2C)cc1COc1cc2c(cn1)[C@H](CC(=O)O)CS2]

Similar procedure to Example 2 was followed to arrive at the title compound, with the LC-MS[M+H]-m/z as 459.

### Example 133

### (S)-2-(6-((2′,6′-dimethyl-4′-nitro-[1,1′-biphenyl]-3-yl)methoxy)-2,3-dihydrothieno[3,2-c]pyridin-3-yl)acetic acid

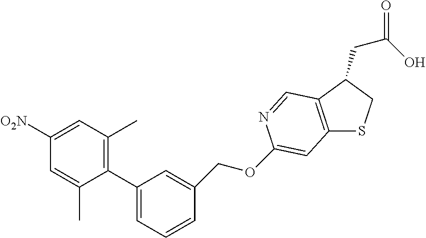 [SMILES: Cc1cc([N+](=O)[O-])cc(C)c1-c1cccc(COc2cc3c(cn2)[C@H](CC(=O)O)CS3)c1]

Similar procedure to Example 2 was followed to arrive at the title compound, with the LC-MS[M+H]-m/z as 451.

### Example 134

### (S)-2-(6-((4′-bromo-2′,6′-dimethyl-[1,1′-biphenyl]-3-yl)methoxy)-2,3-dihydrothieno[3,2-c]pyridin-3-yl)acetic acid

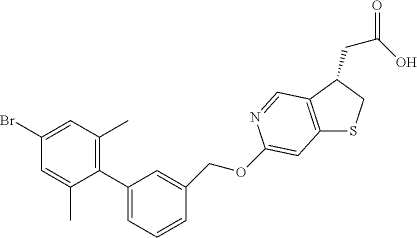 [SMILES: Cc1cc(Br)cc(C)c1-c1cccc(COc2cc3c(cn2)[C@H](CC(=O)O)CS3)c1]

Similar procedure to Example 2 was followed to arrive at the title compound, with the LC-MS[M+H]-m/z as 485.

### Example 135

### (S)-2-(6-((2′,3′,5′,6′-tetramethy-4′-(3-(methylsulfonyl)propoxy)-[1,1′-biphenyl]-3-yl)methoxy)-2,3-dihydrothieno[3,2-c]pyridin-3-yl)acetic acid

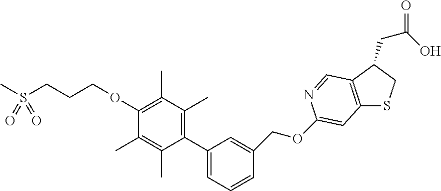 [SMILES: Cc1c(C)c(-c2cccc(COc3cc4c(cn3)[C@H](CC(=O)O)CS4)c2)c(C)c(C)c1OCCCS(C)(=O)=O]

Similar procedure to Example 2 was followed to arrive at the title compound, with the LC-MS[M+H]-m/z as 570.

### Example 136

### (S)-2-(6-((2′-(hydroxymethyl)-6′-methyl-4′-(3-(methylsulfonyl)propoxy)-[1,1′-biphenyl]-3-yl)methoxy)-2,3-dihydrothieno[3,2-c]pyridin-3-yl)acetic acid

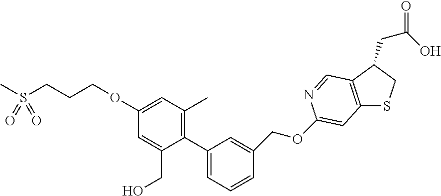 [SMILES: Cc1cc(OCCCS(C)(=O)=O)cc(CO)c1-c1cccc(COc2cc3c(cn2)[C@H](CC(=O)O)CS3)c1]

Similar procedure to Example 2 was followed to arrive at the title compound, with the LC-MS[M+H]-m/z as 558.

### Example 137

### (S)-2-(6-((2′-methoxy-6′-methyl-4′-(3-(methylsulfonyl)propoxy)-[1,1′-biphenyl]-3-yl)methoxy)-2,3-dihydrothieno[3,2-c]pyridin-3-yl)acetic acid

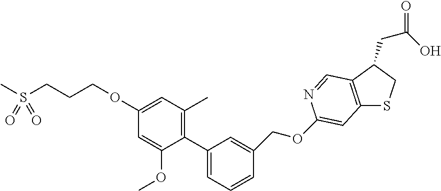 [SMILES: COc1cc(OCCCS(C)(=O)=O)cc(C)c1-c1cccc(COc2cc3c(cn2)[C@H](CC(=O)O)CS3)c1]

Similar procedure to Example 2 was followed to arrive at the title compound, with the LC-MS[M+H]-m/z as 558.

### Example 138

### (S)-2-(6-((4′-(3-(methylsulfonyl)propoxy)-2′,6′-bis(trifluoromethyl)-[1,1′-biphenyl]-3-yl)methoxy)-2,3-dihydrothieno[3,2-c]pyridin-3-yl)acetic acid

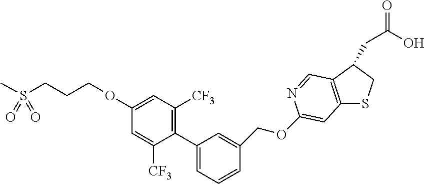 [SMILES: CS(=O)(=O)CCCOc1cc(C(F)(F)F)c(-c2cccc(COc3cc4c(cn3)[C@H](CC(=O)O)CS4)c2)c(C(F)(F)F)c1]

Similar procedure to Example 2 was followed to arrive at the title compound, with the LC-MS[M+H]-m/z as 650.

### Example 139

### (S)-2-(6-((2′,6′-diethyl-4′-(3-(methylsulfonyl)propoxy)-[1,1′-biphenyl]-3-yl)methoxy)-7-(trifluoromethyl)-2,3-dihydrothieno[3,2c]pyridin-3-yl)acetic acid

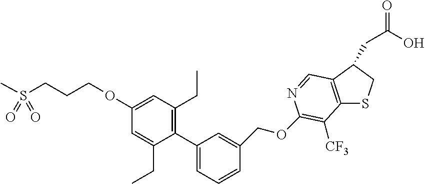 [SMILES: CCc1cc(OCCCS(C)(=O)=O)cc(CC)c1-c1cccc(COc2ncc3c(c2C(F)(F)F)SC[C@H]3CC(=O)O)c1]

Similar procedure to Example 2 was followed to arrive at the title compound, with the LC-MS[M+H]-m/z as 638.

### Example 140

### (S)-2-(6-((4′-(3-(ethylsulfonyl)propoxy)-2′,6′-dimethyl-[1,1′-biphenyl]-3-yl)methoxy)-2,3-dihydrothieno[3,2-c]pyridin-3-yl)acetic acid

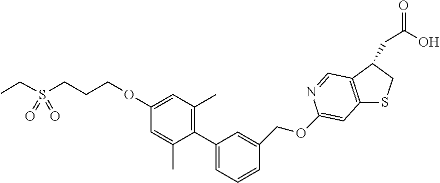 [SMILES: CCS(=O)(=O)CCCOc1cc(C)c(-c2cccc(COc3cc4c(cn3)[C@H](CC(=O)O)CS4)c2)c(C)c1]

Similar procedure to Example 2 was followed to arrive at the title compound, with the LC-MS[M+H]-m/z as 556.

### Example 141

### (S)-2-(6-((2′,6′-diethyl-4′-(3-(methylsulfonyl)propoxy)-[1,1′-biphenyl]-3-yl)methoxy)-2,3-dihydrothieno[3,2-c]pyridin-3-yl)acetic acid

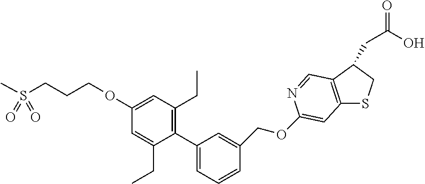 [SMILES: CCc1cc(OCCCS(C)(=O)=O)cc(CC)c1-c1cccc(COc2cc3c(cn2)[C@H](CC(=O)O)CS3)c1]

Similar procedure to Example 2 was followed to arrive at the title compound, with the LC-MS[M+H]-m/z as 570.

### Example 142

### (S)-2-(6-((4′-(3-(methylsulfonyl)propoxy)-[1,1′-biphenyl]-3-yl)methoxy)-2,3-dihydrothieno[3,2-c]pyridin-3-yl)acetic acid

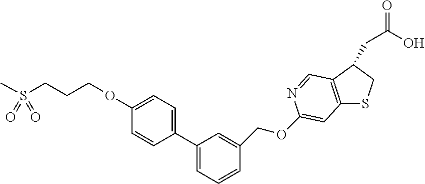 [SMILES: CS(=O)(=O)CCCOc1ccc(-c2cccc(COc3cc4c(cn3)[C@H](CC(=O)O)CS4)c2)cc1]

Similar procedure to Example 2 was followed to arrive at the title compound, with the LC-MS[M+H]-m/z as 514.

### Example 143

### 2-((3S)-6-((4-mesityl-2,3-dihydro-1H-inden-1-yl)oxy)-7-methyl-2,3-dihydrobenzo[b]thiophen-3-yl)acetic acid

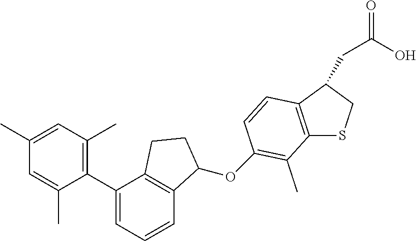 [SMILES: Cc1cc(C)c(-c2cccc3c2CCC3Oc2ccc3c(c2C)SC[C@H]3CC(=O)O)c(C)c1]

Similar procedure to Example 3 was followed to arrive at the title compound, with the LC-MS[M+H]-m/z as 459.

### Example 144

### 2-((3S)-6-((4-(4-methoxy-2,6-dimethylphenyl)-2,3-dihydro-1H-inden-1-yl)oxy)-2,3-dihydrobenzo[b]thiophen-3-yl)acetic acid

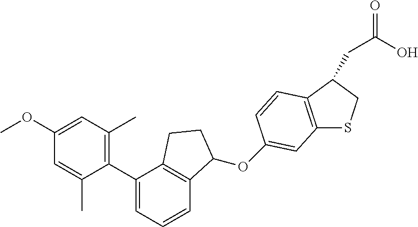 [SMILES: COc1cc(C)c(-c2cccc3c2CCC3Oc2ccc3c(c2)SC[C@H]3CC(=O)O)c(C)c1]

Similar procedure to Example 3 was followed to arrive at the title compound, with the LC-MS[M+H]-m/z as 461.

### Example 145

### 2-((3S)-7-bromo-6-((4-(2,6-dimethyl-4-(trifluoromethyl)phenyl)-2,3-dihydro-1H-inden-1-yl)oxy)-2,3-dihydrobenzo[b]thiophen-3-yl)acetic acid

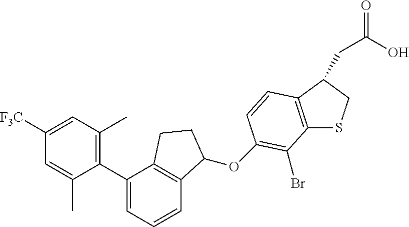 [SMILES: Cc1cc(C(F)(F)F)cc(C)c1-c1cccc2c1CCC2Oc1ccc2c(c1Br)SC[C@H]2CC(=O)O]

Similar procedure to Example 3 was followed to arrive at the title compound, with the LC-MS[M+H]-m/z as 578.

### Example 146

### 2-((3S)-6-((4-(2,6-dimethyl-4-(trifluoromethoxy)phenyl)-2,3-dihydro-1H-inden-1-yl)oxy)-2,3-dihydrobenzo[b]thiophen-3-yl)acetic acid

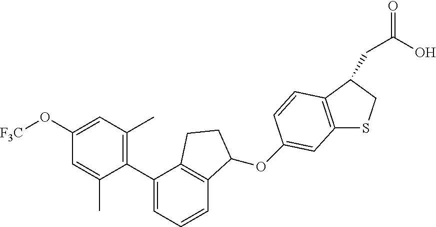 [SMILES: Cc1cc(OC(F)(F)F)cc(C)c1-c1cccc2c1CCC2Oc1ccc2c(c1)SC[C@H]2CC(=O)O]

Similar procedure to Example 3 was followed to arrive at the title compound, with the LC-MS[M+H]-m/z as 515.

### Example 147

### 2-((3S)-6-((4-(4-cyclohexyl-2,6-dimethylphenyl)-6-methyl-2,3-dihydro-1H-inden-1-yl)oxy)-2,3-dihydrobenzo[b]thiophen-3-yl)acetic acid

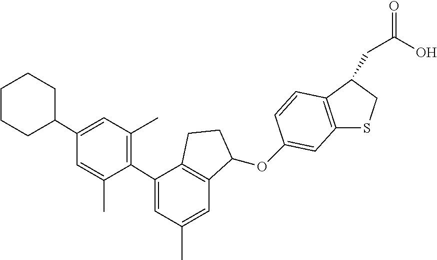 [SMILES: Cc1cc(-c2c(C)cc(C3CCCCC3)cc2C)c2c(c1)C(Oc1ccc3c(c1)SC[C@H]3CC(=O)O)CC2]

Similar procedure to Example 3 was followed to arrive at the title compound, with the LC-MS[M+H]-m/z as 527.

### Example 148

### 2-((3S)-6-((4-(2,6-dimethyl-4-(3-(methylsulfonsyl)propoxy)phenyl)-5-fluoro-2,3-dihydro-1H-inden-1-yl)oxy)-2,3-dihydrobenzo[b]thiophen-3-yl)acetic acid

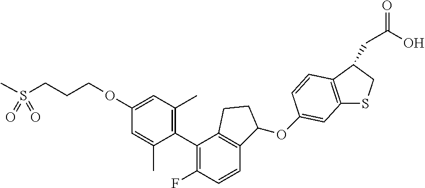 [SMILES: Cc1cc(OCCCS(C)(=O)=O)cc(C)c1-c1c(F)ccc2c1CCC2Oc1ccc2c(c1)SC[C@H]2CC(=O)O]

Similar procedure to Example 3 was followed to arrive at the title compound, with the LC-MS[M+H]-m/z as 585.

### Example 149

### 2-((3S)-6-((4-(2,6-dimethyl-4-(3-(methylsulfonyl)propoxy)phenyl)-5-methoxy-2,3-dihydro-1H-inden-1-yl)oxy)-2,3-dihydrobenzo[b]thiophen-3-yl)acetic acid

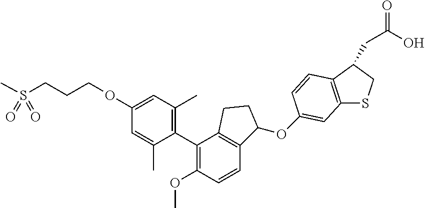 [SMILES: COc1ccc2c(c1-c1c(C)cc(OCCCS(C)(=O)=O)cc1C)CCC2Oc1ccc2c(c1)SC[C@H]2CC(=O)O]

Similar procedure to Example 3 was followed to arrive at the title compound, with the LC-MS[M+H]-m/z as 597.

### Example 150

### 2-((3S)-6-((4-(2,6-dimethyl-4-(3-(methylsulfonyl)propoxy)phenyl)-7-(trifluoromethyl)-2,3-dihydro-1H-inden-1-yl)oxy)-2,3-dihydrobenzo[b]thiophen-3-yl)acetic acid

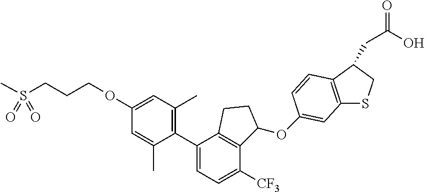 [SMILES: Cc1cc(OCCCS(C)(=O)=O)cc(C)c1-c1ccc(C(F)(F)F)c2c1CCC2Oc1ccc2c(c1)SC[C@H]2CC(=O)O]

Similar procedure to Example 3 was followed to arrive at the title compound, with the LC-MS[M+H]-m/z as 635.

### Example 151

### 2-((3S)-6-((4-(3,5-dichloro-2,6-dimethyl-4-(3-(methylsulfonyl)propoxy)phenyl)-2,3-dihydro-1H-inden-1-yl)oxy)-2,3-dihydrobenzo[b]thiophen-3-yl)acetic acid

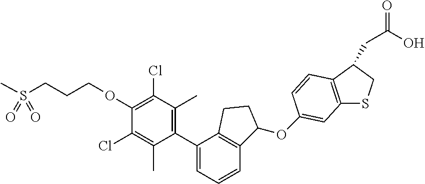 [SMILES: Cc1c(Cl)c(OCCCS(C)(=O)=O)c(Cl)c(C)c1-c1cccc2c1CCC2Oc1ccc2c(c1)SC[C@H]2CC(=O)O]

Similar procedure to Example 3 was followed to arrive at the title compound, with the LC-MS[M+H]-m/z as 636.

### Example 152

### 2-((3S)-6-((4-(3-fluro-2,6-dimethyl-4-(3-(methylsulfonyl)porpoxy)phenyl)-2,3-dihydro-1H-inden-1-yl)oxy)-2,3-dihydrobenzo[b]thiophen-3-yl)acetic acid

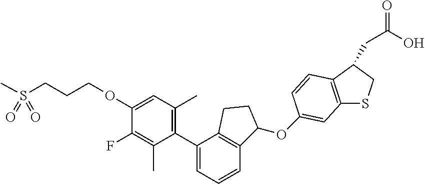 [SMILES: Cc1cc(OCCCS(C)(=O)=O)c(F)c(C)c1-c1cccc2c1CCC2Oc1ccc2c(c1)SC[C@H]2CC(=O)O]

Similar procedure to Example 3 was followed to arrive at the title compound, with the LC-MS[M+H]-m/z as 585.

### Example 153

### 2-((3S)-6-((4-(2-methyl-4-(3-(methylsulfonyl)propoxy)phenyl)-2,3-dihydro-1H-inden-1-yl)oxy)-2,3-dihydrobenzo[b]thiophen-3-yl)acetic acid

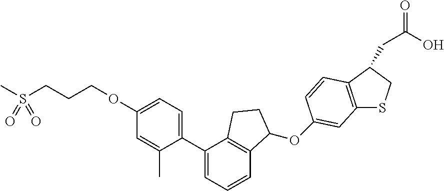 [SMILES: Cc1cc(OCCCS(C)(=O)=O)ccc1-c1cccc2c1CCC2Oc1ccc2c(c1)SC[C@H]2CC(=O)O]

Similar procedure to Example 3 was followed to arrive at the title compound, with the LC-MS[M+H]-m/z as 553.

### Example 154

### 2-((3S)-6-((4-(4-cyano-2,6-dimethylphenyl)-5,7-dimethyl-2,3-dihydro-1H-inden-1-yl)oxy)-2,3-dihydrobenzo[b]thiophen-3-yl)acetic acid

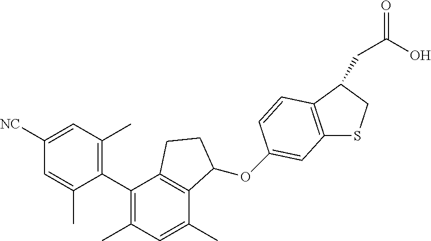 [SMILES: Cc1cc(C#N)cc(C)c1-c1c(C)cc(C)c2c1CCC2Oc1ccc2c(c1)SC[C@H]2CC(=O)O]

Similar procedure to Example 3 was followed to arrive at the title compound, with the LC-MS[M+H]-m/z as 484.

### Example 155

### 2-((3S)-6-((4-(2,6-dimethyl-4-nitrophenyl)-2,3-dihydro-1H-inden-1-yl)oxy)-2,3-dihydrobenzo[b]thiophen-3-yl)acetic acid

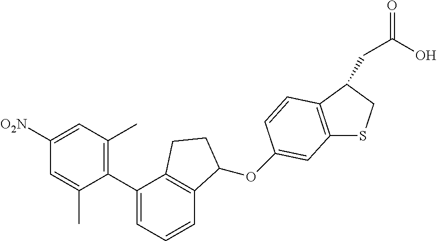 [SMILES: Cc1cc([N+](=O)[O-])cc(C)c1-c1cccc2c1CCC2Oc1ccc2c(c1)SC[C@H]2CC(=O)O]

Similar procedure to Example 3 was followed to arrive at the title compound, with the LC-MS[M+H]-m/z as 476.

### Example 156

### 2-((3S)-6-((4-(4-bromo-2,6-dimethylphenyl)-2,3-dihydro-1H-inden-1-yl)oxy)-2,3-dihydrobenzo[b]thiophen-3-yl)acetic acid

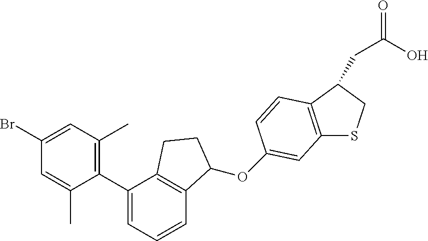 [SMILES: Cc1cc(Br)cc(C)c1-c1cccc2c1CCC2Oc1ccc2c(c1)SC[C@H]2CC(=O)O]

Similar procedure to Example 3 was followed to arrive at the title compound, with the LC-MS[M+H]-m/z as 510.

### Example 157

### 2-((3S)-6-((4-(2,3,5,6-tetramethyl-4-(3-(methylsulfonyl)porpoxy)phenyl)-2,3-dihydro-1H-inden-1-yl)oxy)-2,3-dihydrobenzo[b]thiophen-3-yl)acetic acd

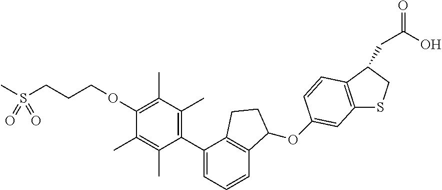 [SMILES: Cc1c(C)c(-c2cccc3c2CCC3Oc2ccc3c(c2)SC[C@H]3CC(=O)O)c(C)c(C)c1OCCCS(C)(=O)=O]

Similar procedure to Example 3 was followed to arrive at the title compound, with the LC-MS[M+H]-m/z as 595.

### Example 158

### 2-((3S)-6-((4-(2-(hydroxymethyl)-6-methyl-4-(3-(methylsulfonyl)propoxy)phenyl)-2,3-dihydro-1H-inden-1-yl)oxy)-2,3-dihydrobenzo[b]thiophen-3-yl)acetic acid

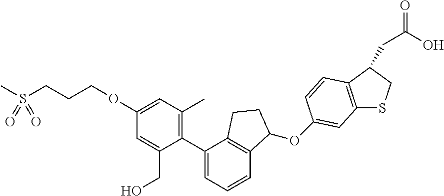 [SMILES: Cc1cc(OCCCS(C)(=O)=O)cc(CO)c1-c1cccc2c1CCC2Oc1ccc2c(c1)SC[C@H]2CC(=O)O]

Similar procedure to Example 3 was followed to arrive at the title compound, with the LC-MS[M+H]-m/z as 583.

### Example 159

### 2-((3S)-6-((4-(2-methoxy-6-methyl-4-(3-(methylsulfonyl)propoxy)phenyl)-2,3-dihydro-1H-inden-1-yl)oxy)-2,3-dihydrobenzo[b]thiophen-3-yl)acetic acid

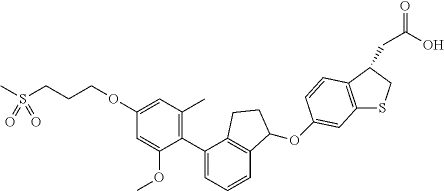 [SMILES: COc1cc(OCCCS(C)(=O)=O)cc(C)c1-c1cccc2c1CCC2Oc1ccc2c(c1)SC[C@H]2CC(=O)O]

Similar procedure to Example 3 was followed to arrive at the title compound, with the LC-MS[M+H]-m/z as 583.

### Example 160

### 2-((3S)-6-((4-(4-(3-(methylsulfonyl)propoxy)-2,6-bis(trifluromenthyl)phenyl)-2,3-dihydro-1H-inden-1-yl)oxy)-2,3-dihydrobenzo[b]thiophen-3-yl)acetic acid

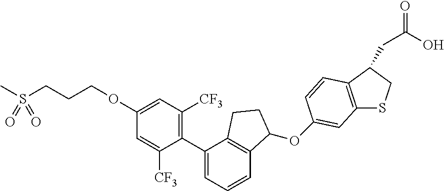 [SMILES: CS(=O)(=O)CCCOc1cc(C(F)(F)F)c(-c2cccc3c2CCC3Oc2ccc3c(c2)SC[C@H]3CC(=O)O)c(C(F)(F)F)c1]

Similar procedure to Example 3 was followed to arrive at the title compound, with the LC-MS[M+H]-m/z as 675.

### Example 161

### 2-((3S)-6-((4-(2,6-dimethyl-4-(3-(methylsulfonyl)propoxy)phenyl)-2,3-dihydro-1H-inden-1-yl)oxy)-7-(trifluoromethyl)-2,3-dihydrobenzo[b]thiophen-3-yl)acetic acid

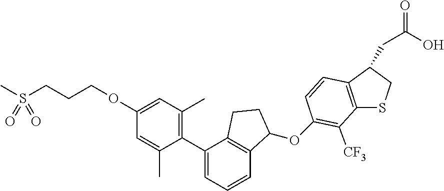 [SMILES: Cc1cc(OCCCS(C)(=O)=O)cc(C)c1-c1cccc2c1CCC2Oc1ccc2c(c1C(F)(F)F)SC[C@H]2CC(=O)O]

Similar procedure to Example 3 was followed to arrive at the title compound, with the LC-MS[M+H]-m/z as 635.

### Example 162

### 2-((S)-6-(((R)-5-(2,6-dimethyl-4-(3-(methylsulfonyl)propoxy)phenyl)-1,2,3,4-tetrahydronaphthalen-1-yl)oxy)-2,3-dihydrobenzo[b]thiophen-3-yl)acetic acid

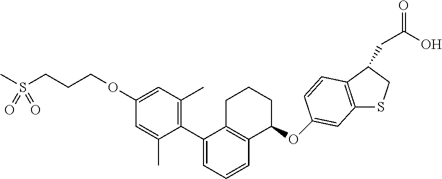 [SMILES: Cc1cc(OCCCS(C)(=O)=O)cc(C)c1-c1cccc2c1CCC[C@H]2Oc1ccc2c(c1)SC[C@H]2CC(=O)O]

Similar procedure to Example 1 was followed to arrive at the title compound, with the LC-MS[M+H]-m/z as 581.

### Example 163

### 2-((S)-6-(((S)-1-(4-(3-methoxypropoxy)-2,6-dimethylphenyl)-6,7,8,9-tetrahydro-5H-benzo[7]annulen-5-yl)oxy)-2,3-dihydrobenzo[b]thiophen-3-yl)acetic acid

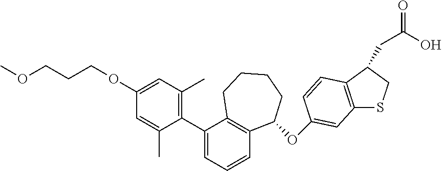 [SMILES: COCCCOc1cc(C)c(-c2cccc3c2CCCC[C@@H]3Oc2ccc3c(c2)SC[C@H]3CC(=O)O)c(C)c1]

Similar procedure to Example 1 was followed to arrive at the title compound, with the LC-MS[M+H]-m/z as 547.

### Example 164

### 2-((3S)-6-((4-(4-(3-(ethylsulfonyl)propoxy)-2,6-dlmethylphenyl)-2,3-dihydro-1H-inden-1-yl)oxy)-2,3-dihydrobenzo[b]thiophen-3-yl)acetic acid

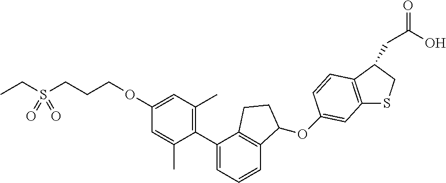 [SMILES: CCS(=O)(=O)CCCOc1cc(C)c(-c2cccc3c2CCC3Oc2ccc3c(c2)SC[C@H]3CC(=O)O)c(C)c1]

Similar procedure to Example 3 was followed to arrive at the title compound, with the LC-MS[M+H]-m/z as 531.

### Example 165

### 2-((3S)-6-((4-(2,6-diethyl-4-(3-(methylsulfonyl)propoxy)phenyl)-2,3-dihydro-1H-inden-1-yl)oxy)-2,3-dihydrobenzo[b]thiophen-3-yl)acetic acid

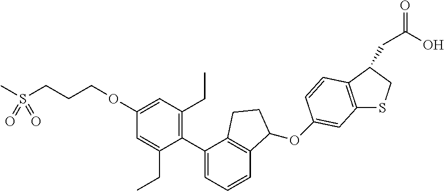 [SMILES: CCc1cc(OCCCS(C)(=O)=O)cc(CC)c1-c1cccc2c1CCC2Oc1ccc2c(c1)SC[C@H]2CC(=O)O]

Similar procedure to Example 3 was followed to arrive at the title compound, with the LC-MS[M+H]-m/z as 595.

### Example 166

### 2-((3S)-6-((4-(4-(3-(methylsulfonyl)propoxy)phenyl)-2,3-dihydro-1H-inden-1-yl)oxy)-2,3-dihydrobenzo[b]thiophen-3-yl)acetic acid

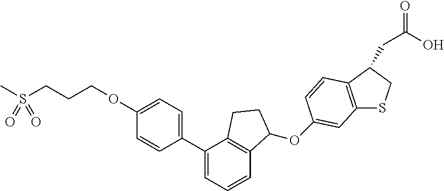 [SMILES: CS(=O)(=O)CCCOc1ccc(-c2cccc3c2CCC3Oc2ccc3c(c2)SC[C@H]3CC(=O)O)cc1]

Similar procedure to Example 3 was followed to arrive at the title compound, with the LC-MS[M+H]-rrs/z as 539.

### Example A

### GPR₄₀ Activation Assay

This test procedure substantially carried out in accordance with the HDB company's Wash Free Fluo-8 Calcium Assay Kits. HEK293 cells stably expressing GPR₄₀ were selected in a media containing Neomycin/G418. The cells were plated at a concentration of 30 uL 15,000 cells per well in black 384-well clear bottom tissue culture treated plates. The cells were incubated for 24 h in a incubator with 5% CO₂.

According to Wash Free Fluo-8 Calcium Assay Kits operating instruction, one component A was dissolved in 210 uL DMSO, one component B was dissolved in 2.4 mL component C. After a 200 mM probenecid solution was prepared, 40 μL component A, 400 μL component B, 200 μL 200 mM probenecid and 19880 μL component C were mixed to a 20 mL test solution.

After removal of medium from the black 384-well dear bottom tissue culture treated plates by centrifuge, the test solution was added at 30 uL/well and the plates were incubated for 60 mins at 37° C. All test compounds were diluted to appropriate concentrations in HBSS buffer. Compounds were added to the cells and the wells were read fluorescence intensity at Ex=494 nm, Em=516 nm and readings were taken for 2 mins with an interval of 1 s. The compounds dose-dependent curve and EC₅₀ were obtained by the Graphpad Prism4 software. The activity test results of some examples of the present invention are shown in the following Table.

| Compound No. | EC₅₀ (μm) |
|---|---|
| 51 | 0.8780 |
| 52 | 1.260 |
| 53 | 0.9971 |

### Example B

### Oral Glucose Tolerance Test

Male ICR mice were fed regular chow and tap water ad libitum with controlled temperature (23° C.), humidity (60%), and lighting (lights on from 7:00 AM to 7:00 PM). Before tests, mice were fasted overnight and first orally given vehicle (0.5% methylcellulose) or test compounds each at a dosage of 60 mg/kg. Sixty minutes later, all animals recelved an oral glucose load (2 g/kg). Blood samples were collected from the tall vein before drug administration, before glucose load (time 0), and 30, 80, 120 mins. After the glucose load, plasma glucose levels were measured on an Accu-Chek glucometer.

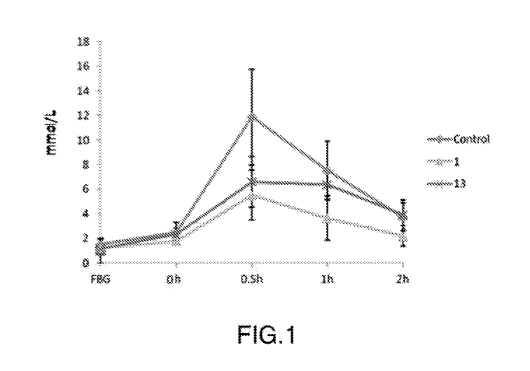

Data were plotted as the mean ±sd. Differences between two groups were analyzed by Student's t test. For the multiple comparisons, differences versus control were tested by Dunnett's test. Results for Example 1 and Example 13 compared to vehicle were shown in FIG. 1.

1. A compound of Formula (I), and pharmaceutical acceptable salts, solvates, isomers, or produgs of the compounds mentioned above, or the mixture of any form above mentioned, 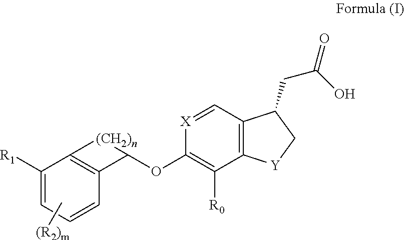
Wherein,
R₁ is selected from the group consisting of an aryl containing k substituents A₁, or a heteroaryl containing k substifuents A₁;
Each A₁ is independently selected from —H, halogen, cyano, nitro, hydroxyl, lower alkyl, substituted lower alkyl, lower alkoxy, substituted lower alkoxy, cycloalkyl, substituted cycloalkyl, alkylsulfonylalkoxy, alkylthioalkoxy, alkoxyalkoxy or 1,1-dioxidotetrahydrothiopyranyloxy;
R₀ is selected from —H, halogen, lower alkyl, lower haloalkyl, substituted lower alkyl, lower alkoxy, substituted lower alkoxy, cycloalkyl, or substituted cycloalkyl;
R₂ is selected from —H, halogen, cyano, nitro, azyl, lower alkyl, lower haloalkyl, lower alkoxy, lower haloalkoxy, C₃-C₂₀ cycloalkyl, C₃-C₂₀ halocycloalkyl, C₆-C₁₄ aryl, C₇-C₁₆ aralkyl, C₆-C₁₄ aryloxy or C₇-C₁₆ aralkyloxy;
X is selected from C or N;
Y is selected from O or S;
m is an integer selected from 0, 1, 2 or 3;
n is an integer selected from 0, 2, 3, 4 or 5, and n is not 0 when both X is C and Y is S;
k is an integer selected from 1, 2, 3, 4 or 5.

2. The compound of claim 1, wherein R₁ is selected from a C₆-C₂₀ aryl containing k substituents A₁, or a C₃-C₂₀ heteroaryl containing k substituents A₁.

3. The compound of claim 2, wherein R₁ is selected from a C₆-C₁₆ aryl containing k substituents A₁, or a C₃-C₂₀ heteroaryl containing k substituents A₁.

4. The compound of claim 3, wherein R₁ is selected from a C₆-C₁₂ aryl containing k substituents A₁, or a C₃-C₁₂ heteroaryl containing k substituents A₁ and 1-4 heteroatoms.

5. The compound of claim 4, wherein R₁ is selected from a phenyl, naphthyl, biphenyl, anthryl, fluorenyl, pyrrolyl, furyl, thienyl, pyridyl, pyranyl, pyrazolyl pyrimidyl, imidazolyl, thiazolyl, Oxazolyl, indolyl, carbazolyl, quinolyl, isoquinolyl, guanine, morpholinyl, piperazinyl, piperidyl, or pyrazinyl, optionally containing k substituents A₁.

6. The compound of claim 5, wherein R₁ is a phenyl containing k substituents A₁.

7. The compound of of claim 1, and pharmaceutically acceptable salts or prodrugs thereof, wherein the compound is a compound of Formula (Ia), 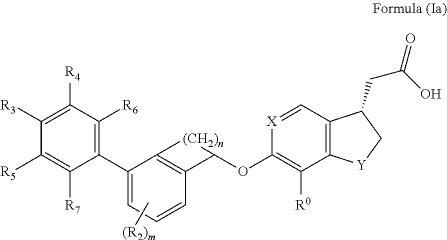
wherein,
R₂ is selected from —H, halogen, cyano, nitro, azyl, lower alkyl, lower haloalkyl, lower alkoxy, lower haloalkoxy, C₃-C₂₀ cycloalkyl, C₃-C₂₀ halocycloalkyl, C₆-C₁₄ aryl, C₇-C₁₆ aralkyl, C₃-C₁₄ aryloxy or C₇-C₁₆ aralkyloxy;
m is an integer selected from 0, 1, 2 or 3;
n is an integer selected from 0, 2, 3, 4 or 5, and n is not 0 when both X is C and Y is S;
R₃ is selected from —H, halogen, cyano, nitro, lower alkyl, substituted lower alkyl, lower alkoxy, substituted lower alkoxy, cycloalkyl, substituted cycloalkyl, alkylsulfonylalkoxy, alkylthio alkoxy, alkoxyalkoxy or 1,1-dioxidotetrahydrothiopyranyloxy;
R₄, R₅, R₆ and R₇ are each independently selected from —H, halogen, cyano, nitro; or hydroxyl, hydroxymethyl, hydroxyethyl, 1-hydroxyethyl, hydroxypropyl, 1-hydroxypropyl 2-hydroxypropyl,
hydroxyisopropyl, methyl, ethyl, propyl, isopropyl, n-butyl, isobutyl, tertbutyl, methoxyl, oxethyl, isopropoxy, butoxy, isobutoxy, tert-butoxy, sec-butoxy, azyl, dimethylamino, methylthio, sulfuryl, acetyl, cyclopropyl, cyclobutyl, cyclopentyl, or cyclohexyl, optionally substituted with —F, —Cl or —Br;
R₀ is selected from —H, halogen, C₁-C₄afkyl, C₁-C₄ haloalkyl, C₁-C₄ alkoxy, C₁-C₄ haloalkoxy, C₃-C₆ cycloalkyl or C₃-C₆ halocycloalkyl.

8. The compound of claim 7, wherein R₃ is selected from —H, halogen, cyano, nitro, C₁-C₆ alkyl, C₁-C₆ haloalkyl, C₁-C₆ alkoxy, C₁-C₆ haloalkoxy, C₃-C₁₂ cycloalkyl, C₃-C₁₂ halocycloalkyl, C₁₋₆-alkylsulfonyl-C₁₋₆ alkoxy, C₁₋₆-alkylthto-C₁₋₆ alkoxy, C₁₋₆-alkoxy-C₁₋₆ alkoxy or 1,1-dioxidotetrahydrothiopyranyloxy.

9. The compound of claim 8, wherein R₅ is selected from —H, halogen, cyano, nitro, C₁C₄alkyl, C₁-C₄ haloalkyl, C₁-C₄ alkoxy, C₁-C₄ haloalkoxy, C₃-C₁₀ cycloalkyl, C₃C₁₀ halocycloalkyl, C₁₋₄-alkylsulfonyl-C₁₋₄ alkoxy; C₁₋₄-alkylthio-C₁₋₄ alkoxy, C₁₋₄-alkoxy-C₁₋₄ alkoxy or 1,1-dioxidtetrahydrothiopyranyloxy.

10. The compound of claim 9, wherein R₃ is selected from —H, halogen, cyano, nitro; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl, cyclohexyl, methylsulfonylmethoxyl, metnylsulfonyloxethyl, methylsulfonylpropoxy, methylsulfonylbutoxy, ethylsulfonylmethoxyl, ethylsulfonyloxethyl, ethylsulfonylpropoxy, ethylsulfonylbutoxy, methylthiomethoxyl, methylthiooxethyl, methylthiopropoxy, methylthiobutoxy, ethylthiomethoxyl, ethylthiooxethyl, ethylthiopropoxy, ethylthiobutoxy, methoxylmethoxyl, methoxyloxethyl, methoxylpropoxy, methoxylbutoxy, oxethylmethoxyl, oxethyloxethyl, oxethylpropoxy, oxethylbutoxy, or 1,1-dioxidotetrahydrothiopyranyloxy, optionally substituted with —F, —Cl or —Br.

11. The compound of claim 10, wherein R₃ is selected from —H, —F, —Cl, —Br, cyano, nitro, methyl, trifluoromethyl, methoxyl, trifluoromethoxy, cyclohexyl, methylsulfonylpropoxy, ethylsulfonylpropoxy, methylthiopropoxy, methoxylpropoxy or 1,1-dioxidotetrahydrothiopyranyloxy.

12. The compound of claim 7, wherein R₅ is selected from —H, halogen, cyano, nitro, azyl, lower alkyl, lower haloalkyl, lower alkoxy, lower haloalkoxy, C₃-C₁₆ cycloalkyl, C₃-C₁₆ halocycloalkyl, C₆-C₁₂ aryl, C₇-C₁₂ aralkyl, C₆-C₁₂ aryloxy or C₇-C₁₂ aralkyloxy.

13. The compound of claim 12, wherein R₂ is selected from —H, halogen, cyano, nitro, azyl lower alkyl, lower haloalkyl, lower alkoxy, lower haloalkoxy, C₃-C₁₂ cycloalkyl, C₃-C₁₂ halocycloalkyl, C₆-C₁₀ aryl, C₇-C₁₀ aralkyl, C₆-C₁₀ aryloxy or C₇C₁₀ aralkyloxy.

14. The compound of claim 13, wherein R₂is selected from —H, halogen, cyano, nitro, azyl, lower alkyl, lower haloalkyl, lower alkoxy, lower haloalkoxy, C₃-C₁₀ cycloalkyl, C₃-C₁₀ halocycloalkyl, C₆-C₁₀ aryl, C₇-C₁₀ aralkyl, C₆-C₁₀ aryloxy or C₇-C₁₀ aralkyloxy.

15. The compound of claim 14, wherein R₂ is selected from —H, halogen, cyano, nitro, azyl; or C₁-C₄ alkyl, C₁-C₄ alkoxy, C₃-C₆ cycloalkyl, C₆-C10 aryl, C₇-C₈ aralkyl, C₆-C₈ aryloxy, or C₇-C₁₀ aralkyloxy, optionally substituted with halogens.

16. The compound of claim 15, wherein R₂ is selected from —H, —F, —Cl, —Br, cyano, nitro, azyl; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl, cyclohexyl, phenyl, indenyl, naphthyl, benzyl, phenemyl, benzyloxy, Phenylethoxy, methylbenzyloxy, ethylbenzyloxy, or propylbenzyloxy, optionally substituted with halogens.

17. The compound of claim 16, wherein R₂ is selected from —H, —F, —Cl, —Br, methyl, trifluoromethyl or methoxyl.

18. The compound of claim 7, wherein R₀ is selected from —H, —F, —Cl, —Br; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl cyclobutyl, cyclopentyl, or cyclohexyl, optionally substituted with halogens.

19. The compound of claim 18, wherein R₀ is selected from —H, —F, —Cl, —Br, methyl or trifluoromethyl.

20. The compound of claim 7, wherein R₃ is selected from —H, halogen, cyano, nitro, C₁-C₆ alkyl, C₁-C₆ haloalkyl, C₁-C₆ alkoxy, C₁-C₆ haloalkoxy, C₃-C₁₂ cycloalkyl, C₃-C₁₂ halocycloalkyl, C₁₋₆-alkylsulfonyl-₁₋₆ alkoxy, C₁₋₆-alkylthio-C₁₋₆ alkoxy, C₁₋₆-alkoxy-C₁₋₆ alkoxy or 1,1-dioxidotetrahydrothiopyranyloxy;
R₂ is selected from —H, halogen, cyano, nitro, azyl, lower alkyl, lower haloalkyl, lower alkoxy, lower haloalkoxy, C₃-C₁₆ cycloalkyl, C₃-C₁₆ halocycloalkyl, C₆-C₁₂ aryl, C₇-C₁₂ aralkyl, C₆-C₁₂ aryloxy or C₇-C₁₂ aralkyloxy;
R₀ selected from —H, —F, —Cl, —Br; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl, or cyclohexyl, optionally substituted with halogens thereof;
R₄, R₅, R₆ and R₇ are each independently selected from —H, halogen, cyano, nitro; or hydroxyl, hydroxymethyl, hydroxyethyl, 1-hydroxyethyl, hydroxypropyl, 1-hydroxypropyl, 2-hydroxypropyl,
hydroxyisopropyl, methyl, ethyl, propyl, isopropyl, n-butyl, isobutyl, tertbutyl, methoxyl, oxethyl, isopropoxy, butoxy, isobutoxy, tert-butoxy, sec-butoxy, azyl, dimethylamino, methylthto, sulfuryl, acetyl, cyclopropyl, cyclobutyl, cyclopentyl, or cyclohexyl, optionally substituted with —F, —Cl or —Br.

21. The compound of claim 20, wherein R₃ is selected from —H, halogen, cyano, nitro, C₁-C₄ alkyl, C₁-C₄ haloalkyl, C₁-C₄alkoxy, C₁-C₄ haloalkoxy, C₃-C₁₀ cycloalkyl, C₃-C₁₀ halocycloalkyl, C₁₋₄-alkylsulfonyl-C₁₋₄ alkoxy, C₁₋₄-alkylthio-C₁₋₄ alkoxy, C₁₋₄-alkoxy-C₁₋₄ alkoxy or 1,1-dioxidotetrahydrothiopyranyloxy;
R₂ is selected from —H, halogen, cyano, nitro, azyl, lower alkyl, lower haloalkyl, lower alkoxy, lower haloalkoxy, C₃-C₁₂ cycloalkyl, C₃-C₁₂ halocycloalkyl, C₆-C₁₀ aryl, C₇-C₁₀ aralkyl, C₆-C₁₀ aryloxy or C₇-C₁₀ aralkyloxy;
R₀ is selected from —H, —F, —Cl, —Br; or methyl, ethyl, propyl, isopropyl, n-butyl, isobutyl tert-butyL sec-butyl, methoxyl, oxethyl, propoxy, isopropoxy, butoxy, isobutoxy, tert-butoxy, sec-butoxy, cyclopropyl, cyclobutyl, cyclopentyl, or cyclohexyl, optionally substituted with halogens;
R₄, R₅, R₆ and R₇ are each independently selected from —H, halogen, cyano, nitro; or hydroxyl, hydroxymethyl, hydroxyethyl, 1-hydroxyethyl, hydroxypropyl, 1-hydroxypropyl, 2-hydroxypropyl,
hydroxyisopropyl, methyl, ethyl, propyl, isopropyl, n-butyl, isobutyl, tertbutyl methoxyl, oxethyl, isopropoxy, butoxy, isobutoxy, tert-butoxy, sec-butoxy, azyl, dimethylamino, methylthio, sulfuryl acetyl, cyclopropyl, cyclobutyl, cyclopentyl, or cyclohexyl, optionally substituted with by —F, —Cl or —Br.

22. The compound of claim 21, wherein R₃ is selected from —H, halogen, cyano, nitro; or methyl, ethyl, propyl, isopropyl, n-butyl, isobutyl, tert-butyl, sec-butyl, methoxyl, oxethyl, propoxy, isopropoxy; butoxy, isobutoxy, tert-butoxy, sec-butoxy, cyclopropyl, cyclobutyl, cyclopentyl, cyclohexyl, methylsulfonylmethoxyl, methylsulfonyloxethyl, methylsulfonylpropoxy, methylsulfonylbutoxy, ethylsulfonylmethoxyl, ethylsulfonyloxethyl, ethylsulfonylpropoxy, ethylsulfonylbutoxy, methylthiomethoxyl, methylthiooxethyl, methylthiopropoxy, methylthiobutoxy, ethylthiomethoxyl, ethylthiooxethyl, ethythiopropoxy, ethylthiobutoxy, methoxylmethoxyl, methoxyloxethyl, methoxylpropoxy, methoxylbutoxy, oxethylmethoxyl, oxethyloxethyL oxethylpropoxy; oxethylbutoxy, or 1,1-dioxidotetrahydrothiopyranyloxy, optionally substituted with —F, —Cl or —Br;
R₂ is selected from —H, halogen, cyano, nitro, azyl; or C₁-C₄ alkyl, C₁-C₄ alkoxy, C₃-C₆ cycloalkyl, C₆-C₁₀ aryl, C₇-C₈ aralkyl, C₆-C₈ aryloxy, or C₇-C₁₀ aralkyloxy, optionally substituted with halogens;
R₀ is selected from —H, —F, —Cl, —Br; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl, or cyclohexyl, optionally substituted with halogens;
R₄, R₅, R₆ and R₇ are each independently selected from —H, halogen, cyano, nitro; or hydroxyl, hydroxymethyl, hydroxyethyl, 1-hydroxyethyl, hydroxypropyl, 1-hydroxypropyl, 2-hydroxypropyl,
Hydroxyisopropyl, methyl, ethyl, propyl, isopropyl, n-butyl, isobutyl, tertbutyl, methoxyl, oxethyl, isopropoxy, butoxy, isobutoxy, tert-butoxy, sec-butoxy, azyl, dimethylamino, methylthio, sulfuryl, acetyl, cyclopropyl, cyclobutyl, cyclopentyl, or cyclohexyl, optionally substituted with —F, —Cl or —Br.

23. The compound of claim 22, wherein R₃ is selected from —H, halogen, cyano, nitro; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy; cyclopropyl, cyclobutyl, cyclopentyl, cyclohexyl, methylsulfonylmethoxyl, methylsulfonyloxethyl, methylsulfonylpropoxy, methylsulfonylbutoxy, ethylsulfonylmethoxyl, ethylsulfonyloxethyl, ethylsulfonylpropoxy, ethylsulfonylbutoxy, methylthiomethoxyl, methylthiooxethyl, methylthiopropoxy, methylthiobutoxy, ethylthiomethoxyl, ethylthiooxethyl, ethylthiopropoxy, ethylthiobutoxy, methoxylmethoxyl, methoxyloxethyl, methoxylpropoxy, methoxylbutoxy, oxethylmethoxyl, oxethyloxethyl, oxethylpropoxy, oxethylbutoxy, or 1,1-dioxidotetrahydrothiopyranyloxy, optionally substituted by —F, —Cl or —Br;
R₂ is selected from —H, —F, —Cl, —Br, cyano, nitro, azyl; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl, cyclohexyl, phenyl, indenyl, naphthyl, benzyl, phenemyl, benzyloxy, Phenylethoxy, methylbenzyloxy, ethylbenzyloxy, or propylbenzyloxy , optionally substituted with halogens;
R₀ is selected from —H, —F, —Cl, —Br; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl, or cyclohexyl, optionally substituted with halogens;
R₄, R₅, R₆ and R₇ are independently selected from —H, halogen, cyano, nitro; or hydroxyl, hydroxymethyl, hydroxyethyl, 1-hydroxyethyl, hydroxypropyl 1-hydroxypropyl, 2-hydroxypropyl,
Hydroxyisopropyl, methyl, ethyl, propyl, isopropyl, n-butyl, isobutyl, tertbutyl, methoxyl, oxethyl, isopropoxy, butoxy, isobutoxy, tert-butoxy, sec-butoxy, azyl, dimethylamino, methylthio, sulfuryl, acetyl, cyclopropyl, cyclobutyl, cyclopentyl, or cyclohexyl, optionally substituted with —F, —Cl or —Br.

24. The compound of claim 23, wherein R₃ is selected from —H, halogen, cyano, nitro; or methyl, ethyl, propyl, isopropyl, n-butyl, isobutyl, tert-butyl, sec-butyl, methoxyl, oxethyl, propoxy, isopropoxy, butoxy, isobutoxy, tert-butoxy, sec-butoxy, cyclopropyl, cyclobutyl, cyclopentyl, cyclohexyl, methylsulfonylmethoxyl, methylsulfonyloxethyl, methylsulfonylpropoxy, methylsulfonylbutoxy, ethylsulfonylmethoxyl, ethylsulfonyloxethyl, ethylsulfonylpropoxy, ethylsulfonylbutoxy, methylthiomethoxyl, methylthiooxethyl, methylthiopropoxy, methylthiobutoxy, ethylthiomethoxyl, ethylthsooxethyl, ethylthsopropoxy, ethylthiobutoxy, methoxylmethoxyl, methoxyloxethyl, methoxylpropoxy, methoxylbutoxy, oxethylmethoxyl, oxethyloxethyl, oxethylpropoxy, oxethylbutoxy, or 1,1-dioxidotetrahydrothiopyranyloxy, optionally substituted with —F, —Cl or —Br;
R₂ is selected from —H, —F, —Cl, —Br, methyl, trifluoromethyl or methoxyl;
R₀ is selected from —H, —F, —Cl, —Br, methyl or trifluoromethyl;
R₄ and R₅ are independently selected from —H, —F, —Cl, —Br or methyl;
R₆ and R₇ are independently selected from —H, methyl, ethyl, trifluoromethyl, methoxyl or hydroxymethyl.

25. The compound of claim 24, wherein n is 2, X is C, Y is S.

26. The compound of claim 24, wherein R₃ is selected from —H, —F, —Cl, —Br, cyano, nitro, methyl, trifluoromethyl, methoxyl, trifluoromethoxy, cyclohexyl, methylsulfonylpropoxy, ethylsulfonylpropoxy, methylthiopropoxy, methoxylpropoxy or 1,1-dioxidotetrahydrothiopyranyloxy;
R₂ is selected from —H, —F, —Cl, —Br, methyl, trifluoromethyl or methoxyl;
R₀ is selected from —H, —F, —Cl, —Br, methyl or trifluoromethyl;
R₄ and R₅ are independently selected from —H, —F, —Cl, —Br or methyl;
R₆ and R₇ are independently selected from —H, methyl, ethyl, trifluoromethyl, methoxyl or hydroxymethyl.

27. The compound of claim 26, wherein n is 0, X is H, Y is O.

28. The compound of claim 26, wherein n is 2, X is N, Y is O.

29. The compound of claim 26, wherein n is 0, X is N, Y is S.

30. The compound of claim 26, wherein n is 2, X is N, Y is S.

31. The compound of claim 26, wherein n is 2, X is C, Y is S.

32. The compound of claim 1, and pharmaceutically acceptable salts or prodrugs thereof, wherein the compound or the prodrug of the compound is selected from:
2-((S)6-(((S)-4-(4-(3-methoxypropoxy)-2,6-dimethylphenyl)-2,3-dihydro-1H-inden-1-yl)oxy)-2,3-dihydrobenzo[b]thiophen-3-yl)acetic acid;
(S)-2-(6-((2′,6′-dimethyl-4′-(3-(methylsulfonyl)poropoxy)-[1,1′-biphenyl]-3-yl)methoxy)-2,3-dihydrofuro[3,2-c]pyridin-3-yl)acetic acid;
2-((3S)-6-((4-(2,6-dimethyl-4-(3-(methylsulfonyl)propoxy)phenyl)-2,3-dihydro-1H-inden-1-yl)oxy)-2,3-dihydrofuro[3,2-c]pyridin-3-yl)acetic acid;
(S)-2-(6-((2′,6′-dimethyl-4′-(3-(methylthio)propoxy)-[1,1′-biphenyl]3-yl)methoxy)-2,3-dihydrofuro[3,2-c]pyridin-3-yl)acetic acid;
2-((3S)-6-((4-(2,6-dimethyl-4-(3-(methylthio)propoxy)phenyl)2,3-dihydro-1H-inden-1-yl)oxy)-2,3-dihydrofuro[3,2-c]pyridin-3-yl)acetic acid;
2-((3S)-6-((4-(4-(3-methylpropoxy)-2,6-dimethylphenyl)-2,3-dihydro-1H-inden-1-yl)oxy)-2,3-dihydrofuro[3,2-c]pyridin-3-y)acetic acid;
(S)-2-(6-((4′-(3-methoxypropoxy)-2′,6′-dimethyl-[1,1′-biphenyl]-3-yl)methoxy)-2,3-dihydrofuro[3,2-c]pyridin-3-yl)acetic acid;
(S)-2-(6-((4′-((1,1′-dioxidotetrahydro-2H-thiopyran-4-yl)oxy)-2′,6′-dimethyl-[1,1′-biphenyl]3-yl)methoxy)-2,3-dihydrofuro[3,2-c]pyridin-3-yl)acetic acid;
2-((3S)-6-((4-(4-((1,1-dioxidotetrahydro-2H-thiopyran-4-yl)oxy)-2,6-dimethylphenyl)-2,3-dihydro-1H-inden-1-yl)oxy)-2,3-dihydrofuro[3,2-c]pyridin-3-yl)acetic acid;
2-((S)-6-(((R)-4-(4-(3-methoxypropoxy)-2,6-dimethylphenyl)-2,3-dihydro-1H-inden-1-yl)oxy)-2,3-dihydrobeno[b]thiophen-3-yl)acetic acid;
Ethyl 2-((S)-6-(((S)-4-(4-(3-methoxypropoxy)-2,6-dimethylphenyl)-2,3-dihydro-1H-inden-1-yl)oxy)-2,3-dihydrobenzo[b]thiophen-3-yl)acetate;
2-((S)-6-(((S)-4-(2,6-dimethyl-4-(3-(methylsulfonyl)propoxy)phenyl)-2,3-dihydro-1H-inden-1-yl)oxy)-2,3-dihydrobenzo[b]thiophen-3-yl)acetic acid;
2-((S)-6-(((R)-4-(2,6-dimethyl-4-(3-(methylsulfonyl)propoxy)phenyl)-2,3-dihydro-1H-inden-1-yl)oxy)-2,3-dihydrobenzo[b]thiophen-3-yl)acetic acid;
(2-((S)-6-(((R)-4-(2,6dimethyl-4-(3-(methylsulfonyl)propoxy)phenyl)-2,3-dihydro-1H-inden-1-yl)oxy)-2,3-dihydrobenzo[b]thiophen-3-yl)acetoxy)methyl pivalate;
(2-((S)-6-(((R)-4-(4-(3-methoxypropoxy)-2,6-dimethylphenyl)-2,3-dihydro-1H-inden-1-yl)oxy)-2,3-dihydrobenzo[b]thiophen-3-yl)acetoxy)methyl isobutyrate;
2-((S)-6-(((S)-4-(4-(3-methoxypropoxy)-2,6-dimethylphenyl)-2,3-dihydro-1H-inden-1-yl)oxy)-2,3-dihydrofuro[3,2-c]pyridin-3-yl)acetic acid;
2-((S)-6-(((R)-4-(2,6-dimethyl-4-(3-methylsulfonyl)propoxy)phenyl)-2,3-dihydro-1H-inden-1-yl)oxy)-2,3-dihydrofuro[(3,2-c]pyridin-3-yl)acetic acid;
2-((3S)-6-((4-(2,6-dimethylphenyl)-2,3-dihydro-1H-inden-1-yl)oxy)-2,3-dihydrofuro[3,2-c]pyridin-3-yl)acetic acid;
(S)-2-(6-((_{2′,6}′-dimethyl-[1,1′-biphenyl]3-yl)methoxy)-2,3-dihydrofuro[3,2c]pyridin-3-yl)acetic acid;
2-((3S)-6-((4-(4,6-dimethylpyrimidin-5-yl)-2,3-dihydro-1H-inden-1-yl)oxy)-2,3-dihydrofuro[3,2-c]pyridin-3-yl)acetic acid;
(S)-2-6-((3-(4,6-dimethylpyridin-5-yl)benzyl)oxy)-2,3-difydrofuro[3,2-c]pyridin-3-acetic acid;
2-((3S)-6-((₄-(2,4-dimethylpyridin-3-yl)-2,3-dihydro-1H-inden-1-yl)oxy)-2,3-dihydrofuro[3,2-c]pyridin-3-yl)acetic acid;
(S)-2-(6-((3-(2,4-dimethylpyridin-3-yl)benzyl)oxy)-2,3-dihydrofuro[3,2c]pyridin-3-yl)acetic acid;
2-((3S)-6-((4-morpholino-2,3-dihydro-1H-inden-1-yl)oxy)-2,3-dihydrofuro[3,2-c]pyridin-3-yl)acetic acid;
(S)-2-(6-((3-molpholinobenzyl)oxy)-2,3dihydrofuro[3,2-c]pridin-3-yl)acetic acid;
2-((3S)-6-((4-(1-methyl-1H-imidazol-2-yl)-2,3-dihydro-1H-inden-1-yl)oxy)-2,3-dhydrofuro[3,2-c]pyridin-3-yl)acetic acid;
(S)-2-(6-((3-(1-methyl-1H-imidazol-2-yl)benzyl)oxy)-2,3-dihydrofuro[3,2-c]pyridin-3-yl)acetic acid;
2-((3S)-6-((4-(2,6-dimethylphenyl)-2,3-dihydro-1H-inden-1-yl)oxy)-2,3-dihydrothieno[3,2-c]pyridin-3-yl)acetic acid;
(S)-2-(6-((2′,6′-dimethyl-[1,1′-biphenyl]-3-yl)methoxy)-2,3-dihydrothieno[3,2-c]pyridin-3-yl)acetic acid;
2-((3S)-6-((4-(4,6-dimethylpyrimidin5-yl)-2,3-dihydro-1H-inden-1-yl)oxy)2,3-dihydrothien[3,2-c]pyridin-3-yl)acetic acid;
(S)-2-(6-((3-(4,6-dimethylpyrimidin-5-yl)benzyl)oxy)-2,3-dihydrothieno[3,2-c]pyridin-3-acetic acid;
2-((3S)-8-((4-(2,4-dimethylpyridin-3-yl)-2,3-dihydro-1H-inden-1-yl)oxy)-2,3-dihydrothieno[3,2-c]pyridin-3-yl)acetic acid;
(S)-2-(6-((3-(2,4-dimethylpyridin-3-yl)benzyl)oxy)-2,3-dihydrothieno[3,2-c]pyridin-3-yl)acetic acid;
2-((3S)-6-((4-morpholino-2,3-dihydro-1H-inden-1-yl)oxy)-2,3-dihydrothieno[3,2-c]pyridin-3-yl)acetic acid;
(S)-2-(6-((3-morpholinobenzyl)oxy)-2,3-dihydrothieno[3,2-c]pyridin-3-yl)acetic acid;
2-((3S)-6-((4-(1-methyl-1H-imidazol-2-yl)-2,3-dihydro-1H-inden-1-yl)oxy)-2,3-dihydrothieno[3,2-c]pyridin-3-yl)acetic acid;
(S)-2-(6-((3-(1-methyl-1H-imidazol-2-yl)benzyl)oxy)-2,3-dihydrothieno[3,2c]pyridin-3-acetic acid;
2-((3S)-6-((4-(2,6-dimethyl-4-(3-(methylsulfonyl)propoxy)phenyl)-2,3-dihydro-1H-inden-1-yl)oxy)-2,3-dihydrothieno[3,2-c]pyridin-3-yl)acetic acid;
(S)-2-(6-((2′,6′-dimethyl-4′-(3-(methylsulfonyl)propoxy)-[1,1′-biphenyl]-3-yl)methoxy)-2,3-dihydrothieno[3,2-c]pyridin-3-yl)acetic acid;
2-((3S)-6-((4-(2,6-dimethyl-4-(3-(methylthio)propoxy)phenyl-2,3-dihydro-1H-inden-1-yl)-2,3-dihydrothieno[3,2-c]pyridin-3-yl)acetic acid;
(S)-2-(6-((2′,6′-dimethyl-4′-(3-(methylthio)propoxy)-[1,1′-biphenyl]-3-yl)methoxy)-2,3-dihydrothieno[3,2-c]pyridin-3-yl)acetic acid;
2-((3S)-6-((4-(4-(3-methoxypropoxy)-2,6-dimethylphenyl)-2,3-dihydro-1H-1-yl)oxy)-2,3-dihydrothieno[3,2-c]pyridin-3-yl)acetic acid;
(S)-2-(6-((4′-(3-methoxypropoxy)-2′,6′-dimethyl-[1,1′-bipheny]-3-yl)methoxy)2,3-dihydrothieno[3,2-c]pyridin-3-yl)acetic acid;
2-((3S)-6-((4-(4-((1,1-dioxidotetrahydro-2H-thiopyran-4-yl)oxy)2,6-dimethylphenyl-2,3-dihydro-1H-inden-1-yl)oxy)-2,3-dihydrothieno[3,2-c]pyridin-3-yl)acetic acid;
(S)-2-(6-((4′-((1,1-dioxidotetrahydro-2H-thiopyran-4-yl)oxy)-2′,6′-dimethyl-[1,1-biphenyl]-3-yl)methoxy)-2,3-dihydrothieno[3,2-c]pyridin-3-yl)acetic acid;
2-((3S)-6-((4-(2,6-dimethylphenyl)-2,3-dihydro-1H-inden-1-yl)oxy)-2,3-dihydrobenzo[b]thiophen-3-yl)acetic acid;
2-((3S)-6-((4-(4,6-dimethylpyrimidin-5-yl)-2,3-dihydro1H-inden-1-yl)oxy)-2,3-dihydrobenzo[b]thiophen-3-yl)acetic acid;
2-((3S)-6-((4-(2,4-dimethylpyrimidin-3-yl)-2,3-dihydro1H-inden-1-yl)oxy)-2,3-dihydrobenzo[b]thiophen-3-yl)acetic acid;
2-((3S)-6-((4-morpholino-2,3-dihydro-1H-inden-1-yl)oxy)-2,3-dihydrobenzo[b]thiophen-3-yl)acetic acid;
2-((3S)-6-((4-(1-methyl-1H-imidazol-2-yl)-2,3-dihydro-1H-inden-1-yl)oxy)-2,3-dihydrobenzo[b]thiophen-3-yl)acetic acid;
2-((3S)-6-((4-(2,6-dimethyl-4-(3-(methylsulfonyl)porpoxy)phenyl)-2,3-dihydro-1H-inden-1-yl)oxy)-2,3-dihydrobenzo[b]thiophen-3-yl)acetic acid;
2-((3S)-6-((4-(2,6-dimethyl-4-(3-(methylthio)propoxy)phenyl)-2,3-dihydro-1H-inden-1yl)oxy)-2,3-dihydrobenzo[b]thiophen-3-yl)acetic acid;
2-((3S)-6-((4-(4-(₃-methoxypropoxy)phenyl)-2,6-dimethylphenyl)-2,3-dihydro-1H-inden-1-yl)oxy)2,3-dihydrobenzo[b]thiophen-3-yl)acetic acid;
2-((3S)-6-((4-(4-((1,1-dioxidotetrahydro-2H-thiopyran-4-yl)oxy)2,6-dimethylphenyl-2,3-dihydro-1H-inden-1-yl)oxy)-2,3-dihydrobenzo[b]thiophen-3-yl)acetic acid;
2-((3S)-6-((4-mesityl-2,3-dihydro-1H-inden-1-yl)oxy)-7-methyl-2,3-dihydrofuro[3,2c]pyridin-3-yl)acetic acid;
2-((3S)-6-((4-(4-methoxy-2,6-dimethylphenyl)-2,3-dihydro-1H-inden-1-yl)oxy)-2,3-dihydrofuro[3,2-c]pyridin-3-yl)acetic add;
2-((3S)-7-bromo-6-((4-(2,6-dimethyl-4-(trifluoromethyl)phenyl)-2,3-dihydro-1H-inden-1-yl)oxy)-2,3-dihydrofuro[3,2-c]pyridin-3-yl)acetic acid;
2-((3S)-6-((4-(2,6-dimethyl-4-(trifluoromethoxy)phenyl)-2,3-dihydro-1H-inden-1-yl)oxy)-2,3-dihydrofuro[3,2-c]pyridin-3-yl)acetic acid;
2-((3S)-6-((4-(4-cyclohexyl-2,6-dimethylphenyl))-6-methyl-2,3-dihydro-1H-inden-1-yl)ozy)-2,3-dihydrofuro[3,2-c]pyridin-3-yl)acetic acid;
2-((3S)-6-((4-(2,6-dimethyl-4-(3-(methylsulfonyl)propoxy)phenyl)-5-fluro-2,3-dihydro-1H-inden-1-yl)oxy)-2,3-dihydrofuro[3,2-c]pyridin-3-yl)acetic acid;
2-((3S)-6-((4-(2,6-dimethyl-4-(3-(methylsulfonyl)propoxy)phenyl)-5-methoxy-2,3-dihydro-1H-inden-1-yl)oxy)-2,3-dihydrofuro[3,2-c]pyridin-3-yl)acetic acid;
2-((3S)-6-((4-(2,6-dimethyl-4-(3-(methylsulfonyl)propoxy)phenyl)-7-(trifluromethyl)-2,3-dihydro-1H-inden-1-yl)oxy)-2,3-dihydrofuro[3,2-c]pyridin-3-yl)acetic acid;
2-((3S)-6-((4-(3,5-dichloro-2,6-dimethyl-4-(3-(methylsulfonyl)propoxy)phenyl)-2,3-dihydro-1H-inden-1-yl)oxy)-2,3-dihydrofuro[3,2-c]pyridin-3-yl)acetic acid;
2-((3R)-6-((4-(3-fluoro-2,6-dimethyl-4-(3-(methylsulfonyl)propoxy)phenyl)-2,3-1H-inden-1-yl)oxy)-2,3-dihydrofuro[3,2-c]pyridin-3-yl)acetic acid;
2-((3R)-6-((4-(2methyl-4-(3-(methylsulfonyl)propoxy)phenyl)-2,3-dihydro-1H-inden-1-yl)oxy)-2,3-dihydrofuro[3,2-c]pyridin-3-yl)acetic acid;
2-((3S)-6-((4-(4-cyano-2,6′-dimethylphenyl)-5,7-dimethyl-2,3-dihydro-1H-inden-1-yl)oxy)-2,3-dihydrofuro[3,2-c]pyridin-3-yl)acetic acid;
2-((3S)-6-((4-(2,6-dimethyl-4-nitrophenyl)-2,3-dihydro-1H-inden-1-yl)oxy)-2,3-dihydrofuro[3,2c]pyridin-3-yl)acetic acid;
2-((3R)-6-((4-(4-bromo-2,6-dimethylphenyl)-2,3-dihydro-1H-inden-1-yl)oxy)-2,3-dihdrofuro[3,2-c]pyridin-3-yl)acetic acid;
2-((3S)-6-((4-(2,3,5,6-tetramethyl-4-(3-(methylsulfonyl)propoxy)phenyl)-2,3-dihydro-1H-inden-1-yl)oxy)-2,3-dihydrofuro[3,2-c]pyridin-3-yl)acetic acid;
2-((3S)-6-((4-(2-(hydroxymethyl)-6-methyl-4-(3-(methylsulfonyl)propoxy)phenyl)-2,3-dihydro-1H-inden-1-yl)oxy)-2,3-dihydrofuro[3,2-c]pyridin-3-yl)acetic acid;
2-((3S)-6-((4-(2-methoxy-6-methyl-4-(3-(methylsulfonyl)propoxy)phenyl)-2,3-dihydro-1H-inden-1-yl)oxy)-2,3-dihydrofuro[3,2-c]pyridin-3-yl)acetic acid;
2-((3S)-6-((4-(4-(3-(methylsulfonyl)propoxy)phenyl)-2,6-bis(trifluromethyl)phenyl)-2,3-dihydro-1H-inden-1-yl)oxy)-2,3-dihydrofuro[3,2-c]pyridin-3-yl)acetic acid;
2-((3R)-6-((4-(2,6-dimethyl-4-(3-(methylsulfonyl)propoxy)phenyl)-2,3-dihydro-1H-inden-1-yl)oxy)-7-(trifluoromethyl)-2,3-dihydrofuro[3-c]pyridin-3-yl)acetic acid;
2-((3R)-6-((4-(4-(3-(ethylsulfonyl)propoxy)-2,6-dimethylphenyl)-2,3-1H-inden-1-yl)oxy)-2,3-dihydrofuro[3,2-c]pyridin-3-yl)acetic acid;
2-((3R)-6-((4-(2,6-diethyl-4-(3-(methylsulfonyl)propoxy)phenyl)-2,3-dihydro-1H-inden-1-yl)oxy)-2,3-dihydrofuro[3-c]pyridin-3-yl)acetic acid;
2-((3R)-6-((4-(4-(3-(methylsulfonyl)propoxy)phenyl)-2,3-dihydro-1H-inden-1-yl)oxy)-2,3-dihydrofuro[3-c]pyridin-3-yl)acetic acid;
(S)-2-(7-methyl-6-((2′,4′,6′-trimethyl-[1,1′-biphenyl]-3-yl)methoxy)-2,3-dihydrofuro[3,2c]pyridin-3-yl)acetic acid;
(S)-2-(6-((4′-methoxy-2′,6′-dimethyl-[1,1-biphenyl]-3-yl)methoxy)-2,3-dihydroforo[32-c]pyridin-3-yl)acetic acid;
(S)-2-(7-bromo-6-((2′,6′-dimethyl-4′-(trifluoromethyl)-[1,1′-biphenyl]-3-yl)methoxy)-2,3-dihydrofuro[3,2-c]pyridin-3-yl)acetic acid;
(S)-2-(6-((2′,6′-dimethyl-4′-(trifluoromethoxy)-[1,1′-biphenyl]-3-yl)methoxy)-2,3-dihydrofuro[3,2-c]pyridin-3-yl)acetic acid;
(S)-2-(6-((4′-cyclohexyl-2′,5,6′-trimiethyl-[1,1′-biphenyl]-3-yl)methoxy)-2,3dihydrofuro[3,2c]pyridin-3-yl)acetic acid;
(S)-2-(6-(6-fluoro-2′,6′-dimethyl-4′-(3(methylsulfonyl)propoxy)-[1,1′-biphenyl]3-methoxy-2,3-3-dihydrofuro[3,2-c]pyridin-3-yl)acetic acid;
(S)-2-(6-((6-methoxy-2′,6′-dimethyl-4′-(3-(methylsulfonyl)propoxy)-[1,1′-biphenyl]-3yl)methoxy)-2,3-dihydrofuro[3,2-c]pyridin-3-yl)acetic acid;
(S)-2-(6-((2′,6′-dimethyl-4′-(3-(methylsulfonyl)propoxy)-4-(trifluoromethyl)-[1,1′-biphenyl]-3-yl)methoxy)-2,3-dihydrofuro[3,2-c]pyridin-3-yl)acetic acid;
(S)-2-(6-((3′,5′-dichloro-2′,6′-dimethyl-4′-(3(methylsulfonyl)propoxy)-[1,1′-biphenyl]3-yl)methoxy)-2,3-dihydrofuro[3,2-c]pyridin-3-yl)acetic acid;
(S)-2-(6-((3′-fluoro-2′,6′-dimethyl-4′-(3-(methylsulfonyl)propoxy)-[1,1′-biphenyl]-3-yl)methoxy-2,3-dihydrofuro[3,2-c]pyridin-3-yl)acetic acid;
(S )-2-(6-((2′-methyl-4′-(3-(methylsulfonyl)propoxy)-[1,1′-biphenyl]3-yl)methoxy)-2,3-dihydrofuro[3,2-c]pyridin-3-yl)acetic acid;
(S)-2-(6-((4′-cyano-2′,4,6,6′-tetramethyl-[1,1′-biphenyl]3-yl)methoxy)-2,3-dihydrofuro[3,2c]pyridin-3-yl)acetic acid;
(S)-2-(6-((2′,6′-dimethyl-4′-nitro-[1,1′-biphenyl]-3-yl)methoxy)-2,3-dihydrofuro[3,2-c]pyridin-3-yl)acetic acid;
(S )-2-(6-((4′-bromo-2,′,6′-dimethyl-[1,1′-bipheny]-3-yl)methoxy)-2,3-dihydrofuro[3,2-c]pyridin-3-yl)acetic acid;
(S)-2-(6-((2′,3+,5′,6′-tetramethyl-4′-(3-(methylsulfonyl)propoxy)-[1,1′-biphenyl]-3-yl)methoxy)-2,3-dihydrofuro[3,2-c]pyridin-3-yl)acetic acid;
(S)-2-(6-((2′-(hydroxymethyl)-6′-methyl-4′-(3-(methylsulfonyl)propoxy)-[1,1′-biphenyl]-3-yl)methoxy)-2,3-dihydrofuro[3,2-c]pyridin-3-yl)acetic acid;
(S)-2-(6-((2′-(methoxy)-6′-methyl-4′-(3-(methylsulfonyl)propoxy)-[1,1′-biphenyl]-3-yl)methoxy)-2,3-dihydrofuro[3,2-c]pyridin-3-yl)acetic acid;
(S)-2-(6-((4′-(3-(methylsulfonyl)propoxy)-2′,6′-bis(trifluoromethyl)-[1,1′-biphenyl]-3-yl)methoxy)-2,3-dihydrofuro[3,2-c]pyridin-3-yl)acetic acid;
(S)-2-(6-((2′,6′-dimethyl-4′-(3-(methylsulfonyl)propoxy)-[1,1′-biphenyl]-3-yl)methoxy)-7-trifluromethyl)2,3-dihydrofuro[3,2-c]pyridin-3-yl)acetic acid;
(S)-2-(6-((4′-(3-(ethylsulfonyl)propoxy)-2′,6′-dimethyl-[1,1′-biphenyl]-3-yl)methoxy)-2,3-dihydrofuro[3,2-c]pyridin-3-yl)acetic acid;
(S)-2-(6-((2′,6′-diethyl-4′-(3-(methylsulfonyl)propoxy)-[1,1′-biphenyl]-3-yl)methoxy)-2,3-dihydrofuro[3,2-c]pyridin-3-yl)acetic acid;
(S)-2-(6-((4′-(3-(methylsulfonyl)propoxy)-[1,1′-biphenyl]-3-yl)methoxy)-2,3-dihydrofuro[3,2-c]pyridin-3-yl)acetic acid;
2-((3S)-6-((4-mesityl-2,3-dihydro-1H-inden-1-yl)oxy)-7-methyl-2,3-dihydrothieno[3,2-]pyridin-3-yl)acetic acid;
(S)-2-(6-((4-(4-methoxy)-2,6-dimethylphenyl)-2,3-dihydro-1H-inden-1-yl)oxy)-2,3-dihydrothieno[3,2-c]pyridin-3-yl)acetic acid; 2,3-dihydrofuro[3,2-c]pyridin-3-yl)acetic acid;
2-((3S)-7-bromo-6-((4-(2,6-dimethyl-4-(trifluromethyl)phenyl)-2,3-dihydro-1H-inden-1-yl)oxy)-2,3-dihydrothieno[3,2-c]pyridin-3-yl)acetic acid;
2-((3S)-6-((4-(2,6-dimethyl-4-(trifluromethoxy)phenyl)-2,3-dihydro-1H-inden-1-yl)oxy)-2,3-dihydrothieno[3,2-c]pyridin-3-yl)acetic acid;
2-((3S)-6-((4-(4-cyclohexyl-2,6-dimethylphenyl)-6-methyl-2,3-dihydro-1H-inden-1-yl)oxy)-2,3-dihydrothieno[3,2-c]pyridin-3-yl)acetic acid;
2-((3S)-6-((4-(2,6-dimethyl-4-(3-(methylsulfonyl)propoxy)phenyl)-5-fluoro-2,3-dihydro-1H-inden-1-yl(oxy)-2,3-dihydrothieno[3,2-c]pyridin-3-yl)acetic acid;
2-((3S)-6-((4-(2,6-dimethyl-4-(3-(methylsulfonyl)propoxy)phenyl-5-methoxy-2,3-dihydro-1H-inden-1-yl)oxy)-2,3-dihydrothieno[3,2-c]pyridin-3-yl)acetic acid;
2-((3S)-6-((4-(2,6-dimethyl-4-(3-(methylsulfonyl)propoxy)phenyl)-7-(trifluoromethyl)-2,3-dihydro-1H-inden-1-yl)oxy)-2,3-dihydrothieno[3,2-c]pyridin-3-yl)acetic acid;
2-((3S)-6-((4-(3,5-dichloro-2,6-dimethyl-4-(3-(methylsulfonyl)propoxy)phenyl)-2,3-dihydro-1H-inden-1-yl)oxy)-2,3-dihydrothieno[3,2-c]pyridin-3-yl)acetic acid;
2-((3S)-6-((4-(3-fluoro-2,6-dimethyl-4-(3-(methylsulfonyl)propoxy)phenyl)-2,3-dihydro-1H-inden-1-yl)oxy)-2,3-dihydrothieno[3,2-c]pyridin-3-yl)acetic acid;
2-((3S)-6-((4-(2-methyl-4-(3-(methylsulfonyl)propoxy)phenyl)-2,3-dihydro-1H-inden-1-yl)oxy)-2,3-dihydrothieno[3,2-c]pyridin-3-yl)acetic acid;
2-((3S)-6-((4-(4-cyano-2,6-dimethylphenyl)-5,7-dimethyl-2,3-dihydro-1H-inden-1-yl)oxy)-2,3-dihydrothieno[3,2-c]pyridin-3-yl)acetic acid;
2-((3S)-6-((4-(2,6-dimethyl-4-nitrophenyl)-2,3-dihydro-1H-inden-1-yl)oxy)-2,3-difydrothieno[3,2-c]pyridin-3-yl)acetic acid;
2-((3S)-6-((4-(4-bromo-2,6-dimethypheyl)-2,3-dihydro-1H-inden-1-yl)oxy)-2,3-dihydrothieno[3,2c]pyridin-3-yl)acetic acid;
2-((3S)-6-((4-(2,3,5,6-tetramethyl-4-(3-(methylsulfonyl)propoxy)phenyl)-2,3-dihydro-1H-inden-1-yl)oxy)-2,3-dihydrothieno[3,2-c]pyridin-3-yl)acetic acid;
2-((3S)-6-((4-(2-(hydroxymethyl)6-methyl-4-(3-(methylsulfonyl)propoxy)phenyl)-2,3-dihydro-1H-inden-1-yl)oxy)-2,3-dihydrothieno[3,2-c]pyridin-3-yl)acetic acid;
2-((3S)-6-((4-(2-methoxy-6-methyl-4-(3-(methylsulfonyl)propoxy)phenyl)-2,3-dihydro-1H-inden-1-yl)oxy)-2,3-dihydrothieno[3,2-c]pyridin-3-yl)acetic acid;
2-((3S)-6-((4-(4-(3-(methylsulfonyl)propoxy)-2,6-bis(trifluoromethyl)phenyl)-2,3-dihydro-1H-inden-1-yl)oxy)-2,3-dihydrothieno[3,2-c]pyridin-3-yl)acetic acid;
2-((3S)-6-((4-(2,6-dimethyl-4-(3-(methylsulfonyl)propoxy)phenyl)-2,3-dihydro-1H-inden-1-yl)oxy)-7-(trifluoromethyl)-2,3-dihydrothieno[3,2-c]pyridin-3-yl)acetic acid;
2-((3S)-6-((4-(4-(3-(ethylsulfonyl)propoxy)-2,6-dimethylhenyl)-2,3-dihydro-1H-inden-1-yl)oxy)-2,-dihydrothieno[3,2-c]pyridin-3-yl)acetic acid;
2-((3S)-6-((4-(2,6-diethyl-4-(3-(methylsulfonyl)propoxy)phenyl)-2,3-dihydro-1H-inden-1-yl)oxy)-2,3-dihydrothieno[3,2-c]pyridin-3-yl)acetic acid;
2-((3S)-6-((4-(4-(3-(methylsulfonyl)propoxy)phenyl)-2,3-dihydro-1H-inden-1-yl)oxy)-2,3-dihydrothieno[3,2-c]pyridin-3-yl)acetic acid;
(S)-2-(7-methyl-6-((2′,4′,66′-trimethyl-[1,1′-biphenyl]3-yl)methoxy)-2,3-dihydrothieno[3,2-c]pyridin-3-yl)acetic acid;
(S)-2-(6-((4′-methoxy-2′,6′-dimethyl-[1,1′-biphenyl]-3-yl)methoxy)-2,3-dihydrothieno[3,2-c]pyridin-3-yl)acetic acid;
(S)-2-(7-bromo-6-((2′,6′-dimethyl-4′-(trifluoromethyl)-[1,1′-biphenyl]-3-yl)methoxy)-2,3-dihydrothieno[3,2-c]pyridin-3-yl)acetic acid;
(S)-2-(6-((2′,6′-dimethyl-4′-(trifluoromethoxy)-[1,1′-biphenyl]-3-yl)methoxy)-2,3-dihydrothieno[3,2-c]pyridin-3-yl)acetic acid;
(S)-2-(6-((4′-cyclohexyl-2′,5,6′-trimethyl-[1,1′-biphenyl]-3-yl)methoxy)-2,3-dihydrothieno[3,2-c]pyridin-3-yl)acetic acid;
(S)-2-(6-((6-fluoro-2′,6′-dimethyl-4′-(3-(methylsulfonyl)propoxy)-[1,1′-biphenyl]-3-dihydrothieno[3,2-c]pyridin-3-yl)acetic acid;
(S)-2-(6-((6-methoxy-2′,6′-dimethyl-4′-(3-(methylsulfonyl)propoxy)-[1,1′-bipheny]3-yl)methoxy)-2,3-dihydrothieno[3,2-c]pyridin-3-yl)acetic acid;
(S)-2-(6-(2′,6′-dimethyl-4′-(3-(methylsulfonyl)propoxy-4-(trifluoromethyl)-[1,1′-biphenyl]-3-yl)methoxy)-2,3-dihydrothieno[3,2-c]pyridin-3-yl)acetic acid;
(S)-2-(6-((3′,5═-dichloro-2′,6′-dimethyl-4′-(methylsulfonyl)propoxy)-[1,1′-biphenyl]-3-yl)methoxy)-2,3-dihydrothieno[3,2-c]pyridin-3-yl)acetic acid;
(S)-2-(6-((3′-fluoro-2′,6′-dimethyl-4′-(3-(methylsulfonyl)propoxy)-[1,1′-biphenyl]-3-yl)methoxy)-2,3-dihydrothieno[3,2-c]pyridin-3-yl)acetic acid;
(S)-2-(6-((2′-methyl-4′-(3-(methylsulfonyl)propoxy)-[1,1′-biphenyl]-3-yl)methoxy)-2,3-dihydrothieno[3,2-c]pyridin-3-yl)acetic acid;
(S)-2-(6-((4′-cyano-2′,4,6,6′-tetramethyl-[1,1′-biphenyl]-3-yl)methoxy)-2,3-dihydrothieno[3,2-c]pyridin-3-yl)acetic acid;
(S)-2-(6-((2′,6′-dimethyl-4′-nitro-[1,1′-biphenyl]3-yl)methoxy)-2,3-dihydrothieno[3,2-c]pyridin-3-yl)acetic acid;
(S)-2-(6′-((4′-bromo-2′,6′-dimethyl-[1,1′-biphenyl]3-yl)methoxy)-2,3-dihydrothieno[3,2-c]pyridin-3-yl)acetic acid;
(S)-2-(6-((2′,3+,5′,6′-tetramethyl-4′-(3-(methylsulfonyl)propoxy)-[1,1′-biphenyl]-3-yl)methoxy)-2,3-dihydrothieno[3,2-c]pyridin-3-yl)acetic acid;
(S)-2-(6-((2′-(hydroxymethyl)-6′-methyl-4′-(3-(methylsulfonyl)propoxy)-[1,1′-biphenyl]-3-yl)methoxy)-2,3-dihydrothieno[3,2-c]pyridin-3-yl)acetic acid;
(S)-2-(6-((2′-methoxy-6′-methyl-4′-(3-(methylsulfonyl)propoxy)-[1,1′-biphenyl]-3-yl)methoxy)-2,3-dihydrothieno[3,2-c]pyridin-3-yl)acetic acid;
(S)-2-(6-((4′-(3-(methylsulfonyl)propoxy)-2′,6′-bis(trifluoromethyl)-[1,1′-biphenyl]-3-yl)methoxy)-2,3-dihydrothieno[3,2-c]pyridin-3-yl)acetic acid;
(S)-2-(6-((2′,6′-diethyl-4′-(3-(methylsulfonyl)propoxy)-[1,1′-biphenyl]-3-yl)methoxy)-7-(trifluoromethyl)-2,3-dihydrothieno[3,2-c]pyridin-3-yl)acetic acid;
(S)-2-(6-((4′-(3-(ethylsulfonyl)propoxy)-2′,6′-dimethyl-[1,1′-biphenyl]-3-yl)methoxy)-2,3-dihydrothieno[3,2-c]pyridin-3-yl)acetic acid;
(S)-2-(6-((2′,6′-diethyl-4′-(3-(methylsulfonyl)propoxy)-[1,1′-biphenyl]-3-yl)methoxy)-2,3-dihydrothieno[3,2-c]pyridin-3-yl)acetic acid;
(S)-2-(6-((4′-(3-(methylsulfonyl)propoxy)-[1,1′-biphenyl]-3-yl)methoxy)-2,3-dihydrothieno[3,2-c]pyridin-3-yl)acetic acid;
2-((3S)-6-((4-mesityl-2,3-dihydro-1H-inden-1-yl)oxy)-7-methyl-2,3-dihydrobenzo[b]thiophen-3-yl)acetic acid;
2-((3S)-6-((4-(4-methoxy-2,6-dimethylphenyl)-2,3-dihydro-1H-inden-1-yl)oxy)-2,3-dihydrobenzo[b]thiophen-3-yl)acetic acid;
2-((3S)-7-bromo-6-((4-(2,6-dimethylphenyl)-2,3-dihydro-1H-inden-1-yl)oxy)-2,3-dihydrobenzo[b]thiophen-3-yl)acetic acid;
2-((3S)-6-((4-(2,6-dimethyl-4-(trifluoromethyl)phenyl)-2,3-dihydro-1H-inden-1-yl)oxy)-2,3-dihydrobenzo[b]thiophen-3-yl)acetic acid;
2-((3S)-6-((4-(4-cyclohexyl-2,6-dimethylphenyl)6-methyl-2,3-dihydro-1H-inden-1-yl)oxy)-2,3-dihydrobenzo[b]thiophen-3-yl)acetic acid;
2-((3S)-6-((4-(2,6-dimethyl-4-(3-(methylsulfonyl)propoxy)phenyl)-5-fluoro-2,3-dihydro-1H-inden-1-yl)oxy)-2,3-dihydrobenzo[b]thiophen-3-yl)acetic acid;
2-((3S)-6-((4-(2,6-dimethyl-4-(3-(methylsulfonyl)propoxy)phenyl)-5-methoxy-2,3-dihydro-1H-inden-1-yl)oxy)-2,3-dihydrobenzo[b]thiophen-3-yl)acetic acid;
2-((3S)-6-((4-(2,6-dimethyl-4-(3-(methylsulfonyl)propoxy)phenyl)-7-fluoro-2,3-dihydro-1H-inden-1-yl)oxy)-2,3-dihydrobenzo[b]thiophen-3-yl)acetic acid;
2-((3S)-6-((4-(3,5-dichloro-4-(3-(methylsulfonyl)propoxy)phenyl)-2,3-dihydro-1H-inden-1-yl)oxy)-2,3-dihydrobenzo[b]thiophen-3-yl)acetic acid;
2-((3S)-6-((₄-(3-fluoro-2,6-dimethyl-4-(3-(methylsulfonyl)propoxy)phenyl)-2,3-dihydro-1H-inden-1-yl)oxy)-2,3-dihydrobenzo[b]thiophen-3-yl)acetic acid;
2-((3S)-6-((4-(3-(methylsulfonyl)propoxy)phenyl)-2,3-dihydro-1H-inden-1-yl)oxy)-2,3-dihydrobenzo[b]thiophen-3-yl)acetic acid;
2-((3S)-6-((4-(4-cyano-2,6-dimethylphenyl)-2,3-dihydro-1H-inden-1-yl)oxy)-2,3-dihydrobenzo[b]thiophen-3-yl)acetic acid;
2-((3S)-6-((4-(2,6-dimethyl-4-nitrophenyl)-2,3-dihydro-1H-inden-1-yl)oxy)-2,3-dihydrobenzo[b]thiophen-3-yl)acetic acid;
2-((3S)-6-((4-(4-bromo-2,6-dimethylphenyl)-2,3-dihydro-1H-inden-1-yl)oxy)-2,3-dihydrobenzo[b]thiophen-3-yl)acetic acid;
2-((3S)-6-((4-(2,3,5,6-tetramethyl-4-(3-(methylsulfonyl)propoxy)phenyl)-2,3-dihydro-1H-inden-1-yl)oxy)-2,3-dihydrobenzo[b]thiophen-3-yl)acetic acid;
2-((3S)-6-((4-(2-(hydroxymethyl)-6-methyl-4-3-(methylsulfonyl)propoxy)phenyl)-2,3-dihydro-1H-inden-1-yl)oxy)-2,3-dihydrobenzo[b]thiophen-3-yl)acetic acid;
2-((3S)-6-((4-(2-methoxy-6-methyl-4-3-(methylsulfonyl)propoxy)phenyl)-2,3-dihydro-1H-inden-1-yl)oxy)-2,3-dihydrobenzo[b]thiophen-3-yl)acetic acid;
2-((3S)-6-((4-(4-(3-(methylsulfonyl)propoxy)-2,6-bis(trifluoromethyl)phenyl)-2,3-dihydro-1H-inden-1-yl)oxy)-2,3-dihydrobenzo[b]thiophen-3-yl)acetic acid;
2-((3S)-6-((4-(2,6-dimethyl-4-(3-(methylsulfonyl)propoxy)phenyl)-2,3-dihydro-1H-inden-1-yl)oxy)-7-(trifluoromethyl-2,3-dihydrobenzo[b]thiophen-3-yl)acetic acid;
2-((S)-6-(((R)-5-(2,6-dimethyl-4-(3-(methylsulfonyl)propoxy)phenyl)-1,2,3,4-tetrahydronaphthalen-1-yl)oxy)-2,3-dihydrobenzo[b]thiophen-3-yl)acetic acid;
2-((S)-6-(((S)-1-(4-(3-methoxypropoxy)-2,6-dimethylphenyl)-6,7,8,9-tetrahydro-5H-benzo[7]annulen-5-yl)oxy)-2,3-dihydrobenzo[b]thiophen-3-yl)acetic acid;
2-((3S)-6-((4-(4-(3-(ethylsulfonyl)propoxy)-2,6dimethylphenyl)-2,3-dihydro-1H-inden-1-yl)oxy)-2,3-dihydrobenzo[b]thiophen-3-yl)acetic acid;
2-((3S)-6-((4-(2,6-diethyl-4-(3-(methylsulfonyl)propoxy)phenyl)-2,3-dihydro-1H-inden-1-yl)oxy)-2,3-dihydrobenzo[b]thiophen-3-yl)acetic acid; or
2-((3S)-6-((4-(4-(3-(methylsulfonyl)propoxy)phenyl)-2,3-dihydro-1H-inden-1-yl)oxy)-2,3-dihydrobenzo[b]thiophen-3-yl)acetic acid.

33. A method of modulating GPR₄₀ receptor function in animals or humans, by administering to the subject a therapeutically effective amount of the compound according to any one of Clalms or 32, or at least one pharmaceutical acceptable salt or prodrug thereof.

34. A method for the treatment and/or prevention of type li diabetes in animals or humans, by administering to the subject a therapeutically effective amount of the compound according to any one of claims 25 or 32, or at least one pharmaceutically acceptable salt or prodrug thereof.

35. A method for the treatment and/or prevention of obesity in animals or humans, by administering to the subject a therapeutically effective amount of the compound according to any one of claims 25 or 32, or at least one pharmaceutically acceptable salt or prodrug thereof.

36. (canceled)

37. (canceled)

38. (canceled)

39. (canceled)

40. A pharmaceutical composition, comprising a therapeutically effective amount of at least one compound according to any one of claims 25 or 32, or at least one pharmaceuticaliy acceptable salt or prodrug thereof, and a pharmaceutically acceptable adjuvant.

41. A method of modulating GPR₄₀ receptor function in animals or humans by administering to the subject a therapeutically effective amount of the pharmaceutical composition of claim 40.

42. A method for the treatment and/or prevention of type II diabetes in animals or humans by administering to the subject a therapeutically effective amount of the pharmaceutical composition of claim 40.

43. A method for the treatment and/or prevention of obesity in animals or humans by administering to the subject a therapeutically effective amount of the pharmaceutical composition of claim 40.

44. (canceled)

45. (canceled)

46. (canceled)

47. (canceled)
